(19) United States
(12) Patent Application Publication
(10) Pub. No.: US 2005/0037429 A1
(43) Pub. Date: Feb. 17, 2005

(54) Artificial receptors including reversibly immobilized building blocks and methods

(75) Inventor: Robert E. Carlson, Minnetonka, MN (US)

Correspondence Address: MERCHANT & GOULD PC, P.O. BOX 2903, MINNEAPOLIS, MN, 55402-0903, US

(73) Assignee: RECEPTORS LLC, CHASKA, MN (US)

(21) Appl. No.: 10/813,612

(22) Filed: Mar. 29, 2004

Related U.S. Application Data
(60) Provisional application No. 60/459,062, filed on Mar. 28, 2003.
(60) Provisional application No. 60/499,776, filed on Sep. 3, 2003.
(60) Provisional application No. 60/499,975, filed on Sep. 3, 2003.
(60) Provisional application No. 60/500,081, filed on Sep. 3, 2003.
(60) Provisional application No. 60/526,511, filed on Dec. 2, 2003.

(51) Int. Cl. G01N 33/53; C12M 1/34

(52) U.S. Cl. 435/7.1; 435/287.2; 427/2.11

(57) ABSTRACT

The present invention relates to artificial receptors, arrays or microarrays of artificial receptors or candidate artificial receptors, methods of and compositions for making them, and methods of using them. Each artificial receptor includes a plurality of building block compounds, which can be mobile or reversibly immobilized on a surface.

### CROSS REFERENCE TO RELATED APPLICATIONS

This application claims priority to the fullest extent to U.S. Provisional Patent Application Ser. No. 60/459,062, filed Mar. 28, 2003; 60/499,776, 60/499,975, and 60/500,081, each filed Sep. 3, 2003; and 60/526,511, filed Dec. 2, 2003.

### INTRODUCTION

The present invention relates to artificial receptors, to methods and compositions for making them, and to methods using them. A receptor provides a binding site for and binds to a ligand. For example, at an elementary level, receptors are often visualized as having a binding site represented as a lock or site into which a key or ligand fits. The binding site is lined with, for example, hydrophobic or functional groups that provide favorable interactions with the ligand.

The present invention provides compositions and methods for forming combinations of building block molecules that make up an artificial receptor. The present artificial receptors include building blocks reversibly immobilized on a support or surface. Reversing immobilization of the building blocks can allow movement of building blocks to a different location on the support or surface, or exchange of building blocks onto and off of the surface.

For example, the combinations of building blocks can bind a ligand when reversibly coupled to or immobilized on the support. Reversing the coupling or immobilization of the building blocks provides opportunity for rearranging the building blocks, which can improve binding of the ligand. Further, the present invention can allow for adding additional or different building blocks, which can further improve binding of a ligand.

FIG. 1 schematically illustrates an embodiment employing an initial artificial receptor surface (A) with four different building blocks on the surface, which are represented by shaded shapes. This initial artificial receptor surface (A) undergoes (1) binding of a ligand to an artificial receptor and (2) shuffling the building blocks on the receptor surface to yield a lead artificial receptor (B). Shuffling refers to reversing the coupling or immobilization of the building blocks and allowing their rearrangement on the receptor surface. After forming a lead artificial receptor, additional building blocks can be (3) exchanged onto and/or off of the receptor surface (C). Exchanging refers to building blocks leaving the surface and entering a solution contacting the surface and/or building blocks leaving a solution contacting the surface and becoming part of the artificial receptor. The additional building blocks can be selected for structural diversity (e.g., randomly) or selected based on the structure of the building blocks in the lead artificial receptor to provide additional avenues for improving binding. The original and additional building blocks can then be (4) shuffled and exchanged to provide higher affinity artificial receptors on the surface (D).

The present artificial receptors and methods can provide unique opportunities for discovering artificial receptors using high throughput screening strategies and then improving upon a lead artificial receptor discovered through the screening. In fact, embodiments of these compositions and methods can allow a lead receptor to improve itself. Although not limiting to the present invention, the reversibly immobilized building blocks can be envisioned as providing equilibrium binding of a test ligand in a system in which the building blocks can be immobilized or mobile.

### BACKGROUND

The preparation of artificial receptors that bind ligands like proteins, peptides, carbohydrates, microbes, pollutants, pharmaceuticals, and the like with high sensitivity and specificity is an active area of research. None of the conventional approaches has been particularly successful; achieving only modest sensitivity and specificity mainly due to low binding affinity.

Antibodies, enzymes, and natural receptors generally have binding constants in the 10⁸-10¹² range, which results in both nanomolar sensitivity and targeted specificity. By contrast, conventional artificial receptors typically have binding constants of about 10³ to 10⁵, with the predictable result of millimolar sensitivity and limited specificity.

Several approaches are being pursued in attempts to achieve highly sensitive and specific artificial receptors. Conventional dynamic combinatorial libraries (DCL) employ ligand and receptor subunits free in bulk solution. With all components free in bulk solution, each receptor subunit is only held in coordination with the ligand by the weak interactions between the individual subunits and the ligand. In DCL, improvement in binding is limited by dissociation of each receptor subunit into the surrounding solution.

Conventional combinatorial methods provide practical access to only hundreds or thousands of different artificial receptors. The present inventor's Combinatorial Artificial Receptor Arrays™ (CARA™) can provide convenient access to one or 2 million different artificial receptors. Convenient access to more than a few million artificial receptors or candidates remains elusive.

There remains a need for practical methods providing access to significant numbers of artificial receptors. Thus, there remains a need for dynamic methods for making artificial receptors, for materials used in such dynamic methods, and for artificial receptors including reversibly immobilized building blocks.

### SUMMARY

The present invention relates to artificial receptors, arrays or microarrays of artificial receptors or candidate artificial receptors, methods of and compositions for making them, and methods of using them. The artificial receptor includes a plurality of building block compounds, which can be mobile or reversibly immobilized on a surface.

The present invention includes a method of making an array of artificial receptors including reversibly immobilized building blocks. This method includes forming a plurality of spots on a solid support. At least certain of the spots include a plurality of building blocks. The method includes reversibly immobilizing building blocks on the solid support in the spots.

The present invention includes a method of making a receptor surface or an artificial receptor. This method includes forming a region on a solid support. The region includes a plurality of building blocks. The method includes reversibly immobilizing building blocks on the solid support in the region.

The invention includes artificial receptors and compositions. The compositions can include a support and a plurality of building blocks. The compositions can also include a functionalized lawn. The functionalized lawn can be coupled to the support. Building blocks can be reversibly immobilized on the support, the lawn, or both. Reversible immobilization can employ any of a variety of reversible interactions, such as van der Waals, hydrophobic, or lipophilic interaction; a covalent bond; a hydrogen bond; an interaction between ions; or the like, or a combination thereof. The building blocks, the support, and or the functionalized lawn can include moieties that can form reversible immobilizing interactions, such as hydrophobic interactions, a covalent bond, a hydrogen bond, an interaction between ions, or the like, or a combination thereof.

In an embodiment, the present invention includes a composition including a surface and a region on the surface. This region includes a plurality of building blocks, at least some of the building blocks being reversibly immobilized on the support.

The present invention includes arrays of artificial receptors and heterogeneous building block arrays. Such an array can include a support and a plurality of building blocks. The array can also include a functionalized lawn. The functionalized lawn can be coupled to the support. The array can also include a plurality of regions on the support. The regions can include a plurality of building blocks. Building blocks can be reversibly immobilized on the support, the lawn, or both.

The present invention includes kits and articles of manufacture. Such an article of manufacture can include a support and a plurality of building blocks. The article of manufacture can also include a functionalized lawn reagent. The functionalized lawn reagent can be configured to be coupled to the support. The plurality of building blocks can be configured to be reversibly coupled to the support, the lawn, or both.

The present invention includes methods of using an artificial receptor. These methods include shuffling building blocks and/or exchanging building blocks. In certain embodiments, shuffling can occur in or on one or more supports, surfaces, compositions, regions, spots or artificial receptors. In certain embodiments, exchanging building blocks can occur onto or off of one or more supports, surfaces, compositions, regions, spots or artificial receptors.

### BRIEF DESCRIPTION OF THE FIGURES

FIG. 1 schematically illustrates an embodiment of the present methods and artificial receptors employing shuffling and exchanging building blocks.

FIG. 2A schematically illustrates an embodiment of an artificial receptor including building blocks reversibly immobilized through hydrophobic interactions with a lawn on a solid support. FIG. 2B schematically illustrates that the building blocks can initially achieve a random distribution on a region of the support and then rearrange. This rearranging can form an improved or lead artificial receptor.

FIG. 3 schematically illustrates an embodiment employing the present artificial receptors to develop a lead artificial receptor using shuffling and exchanging of building blocks.

FIG. 4 schematically illustrates embodiments of the artificial receptor shown in FIG. 2A.

FIGS. 5A and 5B schematically illustrate embodiments of the artificial receptor shown in FIG. 2A.

FIG. 6 schematically illustrates test ligands with 3, 4, 5, 6, 7, or 8 binding surfaces or environments as polygons with 3, 4, 5, 6, 7, or 8 sides. A set of 81 building blocks in groups of 8 can provide up to about 32 billion candidate artificial receptors.

FIG. 7 schematically illustrates serine as a framework for a building block and reactions for derivatizing the building block to add recognition elements.

FIG. 8 schematically illustrates binding space divided qualitatively into 4 quadrants—large hydrophilic, large hydrophobic, small hydrophilic, and small lipophilic.

FIG. 9 illustrates a plot of volume versus logP for 81 building blocks including each of the 9A and 9B recognition elements.

FIGS. 10A and 10B illustrate a plot of volume versus logP for combinations of building blocks with A and B recognition elements forming candidate artificial receptors. FIG. 10B represents a detail from FIG. 10A. This detail illustrates that the candidate artificial receptors fill the binding space evenly.

FIG. 11 illustrates that candidate artificial receptors made up of building blocks can be sorted and evaluated with respect to their nearest neighbors, other candidate artificial receptors made up of one or more of the same building blocks.

FIG. 12 schematically illustrates a false color fluorescence image of a labeled microarray according to an embodiment of the present invention.

FIG. 13 schematically illustrates a two dimensional plot of data obtained for candidate artificial receptors contacted with and/or binding phycoerythrin.

FIG. 14 schematically illustrates a three dimensional plot of data obtained for candidate artificial receptors contacted with and/or binding phycoerythrin.

FIG. 15 schematically illustrates a two dimensional plot of data obtained for candidate artificial receptors contacted with and/or binding a fluorescent derivative of ovalbumin.

FIG. 16 schematically illustrates a three dimensional plot of data obtained for candidate artificial receptors contacted with and/or binding a fluorescent derivative of ovalbumin.

FIG. 17 schematically illustrates a two dimensional plot of data obtained for candidate artificial receptors contacted with and/or binding a fluorescent derivative of bovine serum albumin.

FIG. 18 schematically illustrates a three dimensional plot of data obtained for candidate artificial receptors contacted with and/or binding a fluorescent derivative of bovine serum albumin.

FIG. 19 schematically illustrates a two dimensional plot of data obtained for candidate artificial receptors contacted with and/or binding an acetylated horseradish peroxidase.

FIG. 20 schematically illustrates a three dimensional plot of data obtained for candidate artificial receptors contacted with and/or binding an acetylated horseradish peroxidase.

FIG. 21 schematically illustrates a two dimensional plot of data obtained for candidate artificial receptors contacted with and/or binding a TCDD derivative of horseradish peroxidase.

FIG. 22 schematically illustrates a three dimensional plot of data obtained for candidate artificial receptors contacted with and/or binding a TCDD derivative of horseradish peroxidase.

FIG. 23 schematically illustrates a subset of the data illustrated in FIG. 14.

FIG. 24 schematically illustrates a subset of the data illustrated in FIG. 14.

FIG. 25 schematically illustrates a subset of the data illustrated in FIG. 14.

FIG. 26 schematically illustrates a correlation of binding data for phycoerythrin against logP for the building blocks making up the artificial receptor.

FIG. 27 schematically illustrates a correlation of binding data for phycoerythrin against logP for the building blocks making up the artificial receptor.

FIG. 28 schematically illustrates a two dimensional plot comparing data obtained for candidate artificial receptors contacted with and/or binding phycoerythrin to data obtained for candidate artificial receptors contacted with and/or binding a fluorescent derivative of bovine serum albumin.

FIGS. 29, 30, and 31 schematically illustrate subsets of data from FIGS. 14, 18, and 16, respectively, and demonstrate that the array of artificial receptors according to the present invention yields receptors distinguished between three analytes, phycoerythrin, bovine serum albumin, and ovalbumin.

FIG. 32 schematically illustrates a gray scale image of the fluorescence signal from a scan of a control plate which was prepared by washing off the building blocks with organic solvent before incubation with the test ligand.

FIG. 33 schematically illustrates a gray scale image of the fluorescence signal from a scan of an experimental plate which was incubated with 1.0 μg/ml Cholera Toxin B at 23° C.

FIG. 34 schematically illustrates a gray scale image of the fluorescence signal from a scan of an experimental plate which was incubated with 1.0 μg/ml Cholera Toxin B at 3° C.

FIG. 35 schematically illustrates a gray scale image of the fluorescence signal from a scan of an experimental plate which was incubated with 1.0 μg/ml Cholera Toxin B at 43° C.

FIGS. 36-38 schematically illustrate plots of the fluorescence signals obtained from the candidate artificial receptors illustrated in FIGS. 33-35.

FIG. 39 schematically illustrate plots of the fluorescence signals obtained from the combinations of building blocks employed in the present studies, when those building blocks are covalently linked to the support. Binding was conducted at 23° C.

FIG. 40 schematically illustrates a graph of the changes in fluorescence signal from individual combinations of building blocks at 4° C., 23° C., or 44° C.

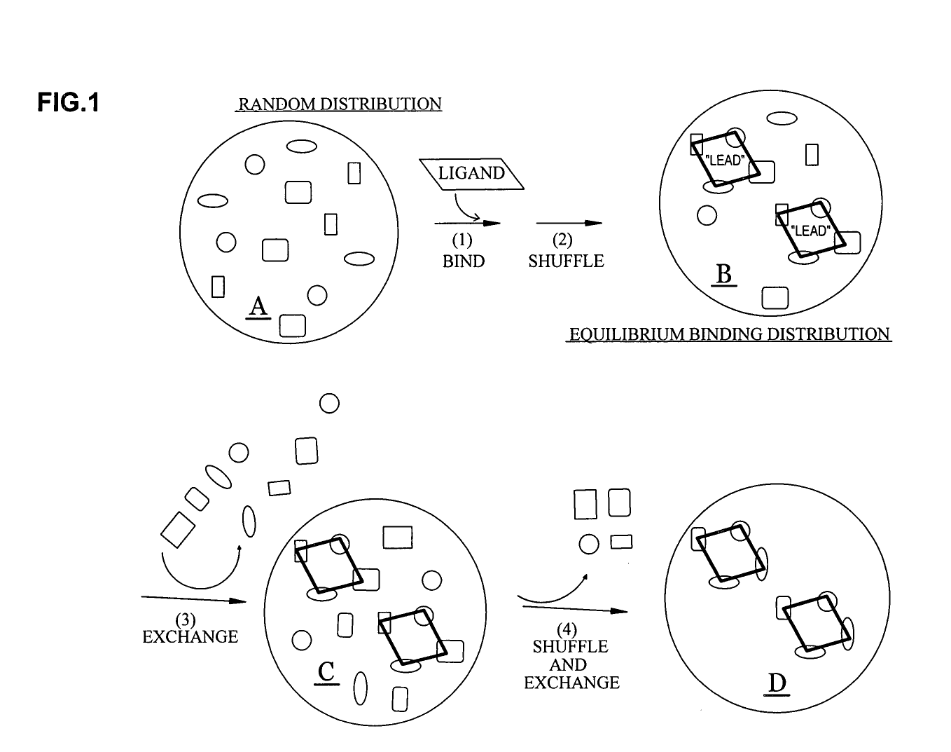

### DETAILED DESCRIPTION

Definitions

A combination of building blocks immobilized on, for example, a support can be a candidate artificial receptor, a lead artificial receptor, or a working artificial receptor. That is, a spot on a slide including a plurality of building blocks or a plurality of building blocks coated on a tube, well, slide, or the like can be a candidate artificial receptor, a lead artificial receptor, or a working artificial receptor. A candidate artificial receptor can become a lead artificial receptor, which can become a working artificial receptor.

As used herein the phrase “candidate artificial receptor” refers to a combination including one or more reversibly immobilized building blocks that can be tested to determine whether or not a particular test ligand binds to that combination.

As used herein the phrase “lead artificial receptor” refers to a combination including one or more reversibly immobilized building blocks that binds a test ligand at a predetermined concentration of test ligand, for example at 10, 1, 0.1, or 0.01 μg/ml, or at 1, 0.1, or 0.01 ng/ml.

As used herein the phrase “working artificial receptor” refers to a combination including one or more reversibly immobilized building blocks that binds a test ligand with a selectivity and/or sensitivity effective for categorizing or identifying the test ligand. That is, binding to that combination including one or more reversibly immobilized building blocks describes the test ligand as belonging to a category of test ligands or as being a particular test ligand. A working artificial receptor can, for example, bind the ligand at a concentration of, for example, 100, 10, 1, 0.1, 0.01, or 0.001 ng/ml.

As used herein the phrase “working artificial receptor complex” refers to a plurality of artificial receptors, each a combination including one or more reversibly immobilized building blocks, that binds a test ligand with a pattern of selectivity and/or sensitivity effective for categorizing or identifying the test ligand. That is, binding to the several receptors of the complex describes the test ligand as belonging to a category of test ligands or as being a particular test ligand. The individual receptors in the complex can each bind the ligand at different concentrations or with different affinities. For example, the individual receptors in the complex can each bind the ligand at concentrations of 100, 10, 1, 0.1, 0.01 or 0.001 ng/ml.

As used herein, the term “building block” refers to a molecular component of an artificial receptor including portions that can be envisioned as or that include, one or more linkers, one or more frameworks, and one or more recognition elements. In an embodiment, the linker includes a moiety suitable for reversibly immobilizing the building block, for example, on a support, surface or lawn. The building block interacts with the ligand.

As used herein, the term “linker” refers to a portion of or functional group on a building block that can be employed to or that does (e.g., reversibly) couple the building block to a support, for example, through covalent link, ionic interaction, or hydrophobic interaction.

As used herein, the term “framework” refers to a portion of a building block including the linker or to which the linker is coupled and to which one or more recognition elements are coupled.

As used herein, the term “recognition element” refers to a portion of a building block coupled to the framework but not covalently coupled to the support. Although not limiting to the present invention, the recognition element can provide or form one or more groups, surfaces, or spaces for interacting with the ligand.

As used herein, the phrase “plurality of building blocks” refers to two or more building blocks of different structure in a mixture, in a kit, or on a support or scaffold. Each building block has a particular structure, and use of building blocks in the plural, or of a plurality of building blocks, refers to more than one of these particular structures. Building blocks or plurality of building blocks does not refer to a plurality of molecules each having the same structure.

As used herein, the phrase “combination of building blocks” refers to a plurality of building blocks that together are in a spot, region, or a candidate, lead, or working artificial receptor. A combination of building blocks can be a subset of a set of building blocks. For example, a combination of building blocks can be one of the possible combinations of 2, 3, 4, 5, or 6 building blocks from a set of N (e.g., N=10-200) building blocks.

As used herein, the term “naïve” used with respect to one or more building blocks refers to a building block that has not previously been determined or known to bind to a test ligand of interest. For example, the recognition element(s) on a naïve building block has not previously been determined or known to bind to a test ligand of interest. A building block that is or includes a known ligand (e.g., GM1) for a particular protein (test ligand) of interest (e.g., cholera toxin) is not naïve with respect to that protein (test ligand).

As used herein, the term “immobilized” used with respect to building blocks coupled to a support refers to building blocks being stably oriented on the support so that they do not migrate on the support or release from the support. Building blocks can be immobilized by covalent coupling, by ionic interactions, such as ion pairing, or by hydrophobic interactions, such as van der Waals interactions.

As used herein, the term “lawn” refers to a layer, spot, or region of functional groups on a support, which can be at a density sufficient to place coupled building blocks in proximity to one another. The functional groups can include groups capable of forming covalent, ionic, or hydrophobic interactions with building blocks. One portion or region of the support can be modified with a first lawn and another (e.g., second) portion or region can be modified with a second lawn.

Artificial Receptors With Reversibly Immobilized Building Blocks

Methods of Making Artificial Receptors

The present invention includes a method of producing an artificial receptor or a candidate artificial receptor. Producing an artificial receptor can include making an array of reversibly immobilized building blocks. Such a method can include forming a plurality of spots or regions on a support. At least some of the spots or regions in the array include a plurality of building blocks. According to the present invention, the method includes reversibly immobilizing the plurality of building blocks on the support.

Reversibly immobilizing building blocks on a support couples the building blocks to the support through a mechanism that allows the building blocks to be uncoupled from the support without destroying or unacceptably degrading the building block or the support. That is, immobilization can be reversed without destroying or unacceptably degrading the building block or the support. In an embodiment, immobilization can be reversed with only negligible or ineffective levels of degradation of the building block or the support. Reversible immobilization can employ readily reversible covalent bonding or noncovalent interactions. Suitable noncovalent interactions include interactions between ions, hydrogen bonding, van der Waals interactions, and the like. Readily reversible covalent bonding refers to covalent bonds that can be formed and broken under conditions that do not destroy or unacceptably degrade the building block or the support.

In an embodiment, reversible immobilization of a building block employs a support functionalized to provide moieties on the support that can engage in a reversible interaction with the building block. In an embodiment, the support can be functionalized with moieties that can engage in reversible covalent bonding, moieties that can engage in noncovalent interactions, a mixture of these moieties, or the like.

The present invention can employ any of a variety of the numerous known functional groups, reagents, and reactions for forming reversible covalent bonds. Suitable reagents for forming reversible covalent bonds include those described in Green, TW; Wuts, PGM (1999), Protective Groups in Organic Synthesis Third Edition, Wiley-Interscience, New York, 779 pp. For example, the support can include functional groups such as a carbonyl group, a carboxyl group, a silane group, boric acid or ester, an amine group (e.g., a primary, secondary, or tertiary amine, a hydroxylamine, a hydrazine, or the like), a thiol group, an alcohol group (e.g., primary, secondary, or tertiary alcohol), a diol group (e.g., a 1,2 diol or a 1,3 diol), a phenol group, a catechol group, or the like. These functional groups can form groups with reversible covalent bonds, such as ether (e.g., alkyl ether, silyl ether, thioether, or the like), ester (e.g., alkyl ester, phenol ester, cyclic ester, thioester, or the like), acetal (e.g., cyclic acetal), ketal (e.g., cyclic ketal), silyl derivative (e.g., silyl ether), boronate (e.g., cyclic boronate), amide, hydrazide, imine, carbamate, or the like. Such a functional group can be referred to as a covalent bonding moiety, e.g., a first covalent bonding moiety.

A carbonyl group on the functionalized support and an amine group on a building block can form an imine or Schiff's base. The same is true of an amine group on the functionalized support and a carbonyl group on a building block. The imine or Schiff's base can be formed and cleaved under conditions that do not destroy or unacceptably degrade either the support or the building block.

A carbonyl group on the functionalized support and an alcohol group on a building block can form an acetal or ketal. The same is true of an alcohol group on the functionalized support and a carbonyl group on a building block. The acetal or ketal can be formed and cleaved under conditions that do not destroy or unacceptably degrade either the support or the building block.

A thiol (e.g., a first thiol) on the functionalized support and a thiol (e.g., a second thiol) on the building block can form a disulfide. The disulfide bond can be formed and cleaved under conditions that do not destroy or unacceptably degrade either the support or the building block.

A carboxyl group on the functionalized support and an alcohol group on a building block can form an ester. The same is true of an alcohol group on the functionalized support and a carboxyl group on a building block. Any of a variety of alcohols and carboxylic acids can form esters that provide covalent bonding that can be reversed in the context of the present invention. For example, readily reversible ester linkages can be formed from alcohols such as phenols with electron withdrawing groups on the aryl ring, other alcohols with electron withdrawing groups acting on the hydroxyl-bearing carbon, other alcohols, or the like; and/or carboxyl groups such as those with electron withdrawing groups acting on the acyl carbon (e.g., nitrobenzylic acid, R—CF₂—COOH, R—CCl₂—COOH, and the like), other carboxylic acids, or the like. Reversible ester linkages can be formed and cleaved under conditions that do not destroy or unacceptably degrade either the support, the lawn, or the building block.

In an embodiment, the support can be functionalized with moieties that can engage in noncovalent interactions. For example, the support can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like. A cationic group on the functionalized support and an anionic group on a building block can form an ionic bond under conditions that do not destroy or unacceptably degrade either the support or the building block. The same is true of an anionic group on the functionalized support and a cationic group on a building block. By way of further example, an 18 carbon alkyl group on the functionalized support and a complementary lipophilic group on a building block can engage in a lipophilic interaction under conditions that do not destroy or unacceptably degrade either the support or the building block. The support can include a plurality of different moieties that can engage in assorted covalent or non-covalent interactions.

In an embodiment, the present methods and compositions can employ a support or substrate including a charged moiety (e.g., a first charged moiety). Suitable charged moieties include positively charged moieties and negatively charged moieties. Suitable positively charged moieties (e.g., at neutral pH in aqueous compositions) include amines, quaternary ammonium moieties, sulfonium, phosphonium, ferrocene, or the like. A positively charged moiety, such as a quaternary ammonium moiety, can also include one or more lipophilic moieties. Suitable negatively charged moieties (e.g., at neutral pH in aqueous compositions) include carboxylates, alkoxylates, phenols substituted with strongly electron withdrawing groups (e.g., tetrachlorophenols), phosphates, phosphonates, phosphinates, sulphates, sulphonates, thiocarboxylates, hydroxamic acids, or the like.

In an embodiment, the present methods and compositions can employ a support including groups that can hydrogen bond (e.g., a first hydrogen bonding group), either as donors or acceptors. The support can include a surface or region with groups that can hydrogen bond. For example, the support can include a surface or region including one or more carboxyl groups, amine groups, hydroxyl groups, carbonyl groups, or the like. Ionic groups can also participate in hydrogen bonding.

In an embodiment, the present methods and compositions can employ a support including a lipophilic moiety (e.g., a first lipophilic moiety). Suitable lipophilic moieties include branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. A lipophilic moiety like a quaternary ammonium lipophilic moiety can also include a positive charge. In an embodiment the lipophilic includes or is a lipid, such as a phospholipid. In an embodiment, the lipophilic moiety includes or is a 16-carbon aliphatic moiety.

In an embodiment, reversible immobilization of a building block employs a support functionalized with a lawn reagent (e.g., a functionalized lawn reagent). The method can include coupling the lawn reagent to the support in, for example, a spot or region. The functionalized lawn reagent can provide functional groups that couple to the support plus moieties that engage in a reversible interaction with the building block. In an embodiment, the functionalized lawn reagent includes moieties that can engage in reversible covalent bonding, moieties that can engage in noncovalent interactions, mixtures of such moieties, or the like.

The functionalized lawn of the present invention can employ any of a variety of the numerous known functional groups, reagents, and reactions for forming reversible covalent bonds. Suitable reagents for forming reversible covalent bonds include those described in Green, TW; Wuts, PGM supra, and the others described above for supports. Such a functional group can be referred to as a covalent bonding moiety, e.g., a first covalent bonding moiety.

A carbonyl group on the functionalized lawn and an amine group on a building block can form an imine or Schiff's base. The same is true of an amine group on the functionalized lawn and a carbonyl group on a building block. The imine or Schiff's base can be formed and cleaved under conditions that do not destroy or unacceptably degrade either the support, the lawn, or the building block.

A carbonyl group on the functionalized lawn and an alcohol group on a building block can form an acetal or ketal. The same is true of an alcohol group on the functionalized lawn and a carbonyl group on a building block. The acetal or ketal can be formed and cleaved under conditions that do not destroy or unacceptably degrade either the support, the lawn, or the building block.

A thiol (e.g., a first thiol) on the functionalized lawn and a thiol (e.g., a second thiol) on a building block can form an disulfide. The disulfide bond can be formed and cleaved under conditions that do not destroy or unacceptably degrade either the support, the lawn, or the building block.

A carboxyl group on the functionalized lawn and an alcohol group on a building block can form an ester. The same is true of an alcohol group on the functionalized lawn and a carboxyl group on a building block. Reversible ester linkages can be formed from alcohols and carboxyl groups described hereinabove. The reversible ester linkages can be formed and cleaved under conditions that do not destroy or unacceptably degrade either the support, the lawn, or the building block.

In an embodiment, the lawn reagent can be functionalized with moieties that can engage in noncovalent interactions. For example, the lawn reagent can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like. A cationic group on the functionalized lawn and an anionic group on a building block can form an ionic bond under conditions that do not destroy or unacceptably degrade either the support or the building block. The same is true of an anionic group on the functionalized support and a cationic group on a building block. By way of further example, an 18 carbon alkyl group on the functionalized lawn and a complementary lipophilic group on a building block can engage in a lipophilic interaction under conditions that do not destroy or unacceptably degrade either the support or the building block. The lawn can include a plurality of different moieties that can engage in assorted covalent or non-covalent interactions.

In an embodiment, the present methods and compositions can employ a lawn reagent including a charged moiety (e.g., a first charged moiety). Suitable charged moieties include positively charged moieties and negatively charged moieties. Suitable positively charged moieties include those described hereinabove. Suitable negatively charged moieties (e.g., at neutral pH in aqueous compositions) include those described hereinabove.

In an embodiment, the present methods and compositions can employ a building block including a charged moiety (e.g., a second charged moiety) that can interact with the lawn or support. Suitable charged moieties include those listed for lawn reagents.

In an embodiment, the present methods and compositions can employ a lawn reagent including a group that can hydrogen bond, either as donors or acceptors (e.g., a first hydrogen bonding group). Suitable hydrogen bonding groups include those described hereinabove. Ionic groups can also participate in hydrogen bonding.

In an embodiment, the present methods and compositions can employ a building block including a group that can hydrogen bond to the lawn or support (e.g., a second hydrogen bonding group). Suitable hydrogen bonding group include those listed for lawn reagents.

In an embodiment, the present methods and compositions can employ lawn reagent including a lipophilic moiety (e.g., a first lipophilic moiety). Suitable lipophilic moieties include those described hereinabove. In an embodiment, the lawn reagent includes a lipophilic moiety (e.g., a first lipophilic moiety) and a covalent bonding moiety (e.g., a first covalent bonding moiety). In an embodiment, the lawn reagent includes a lipophilic moiety (e.g., a first lipophilic moiety) and a charged moiety (e.g., a first charged moiety).

In an embodiment, the present methods and compositions can employ a building block including a lipophilic moiety (e.g., a second lipophilic moiety). Suitable lipophilic moieties include those described hereinabove. In an embodiment, the building block includes a lipophilic moiety (e.g., a second lipophilic moiety) and a covalent bonding moiety (e.g., a second covalent bonding moiety). In an embodiment, the building block includes a lipophilic moiety (e.g., a second lipophilic moiety) and a charged moiety (e.g., a second charged moiety).

In an embodiment, the lawn reagent includes a lipophilic moiety (e.g., a first lipophilic moiety) and a covalent bonding moiety (e.g., a first covalent bonding moiety) and the building block includes a lipophilic moiety (e.g., a second lipophilic moiety) and a covalent bonding moiety (e.g., a second covalent bonding moiety); the lawn reagent includes a lipophilic moiety (e.g., a first lipophilic moiety) and a charged moiety (e.g., a first charged moiety) and the building block includes a lipophilic moiety (e.g., a second lipophilic moiety) and a charged moiety (e.g., a second charged moiety); or combination thereof.

In an embodiment the present method of making an artificial receptor includes a method of making a receptor surface. Such a method can include forming a region on a solid support. The region can include a plurality of building blocks. The method can also include reversibly immobilizing the plurality of building blocks on the solid support in the region. In an embodiment, the present method of making an artificial receptor includes forming a region on a support that includes a plurality of building blocks. This embodiment can also include reversibly immobilizing the plurality of building blocks on the support in the region. The region can be a spot. These embodiments can include mixing the plurality of building blocks and employing the mixture in forming the plurality of spots, regions, or the receptor surface.

In an embodiment the present methods and compositions include reversibly and irreversibly coupled building blocks. For example, the present method can also include irreversibly coupling one or more building blocks to the support. In an embodiment, such an irreversibly coupled building block can be coupled through a covalent bond that cannot be broken without destroying or unacceptably degrading the building block or the support. In an embodiment, irreversible coupling employs a covalent bond that is stable under conditions used to reverse the reversible covalent bond. In an embodiment, an amide bond irreversibly couples a building block to a support.

In an embodiment, a building block reversibly coupled to a lawn or support can be irreversibly coupled to the support. For example, the present method can include converting a reversible covalent bond that links a building block to a lawn or support into an irreversible bond. Such conversions include converting a disulfide link to an irreversible link by, for example, methods including reducing and/or oxidizing the disulfide to an irreversible bond. Such a conversion can include reducing an imine to an amine. In an embodiment, a building block reversibly immobilized on the support through a noncovalent interaction can be irreversibly covalently linked through a photochemical reaction. Such a photochemical reaction can include a photochemically reactive group on the building block reacting with the lawn or support. Such a photochemical reaction can include a photochemically reactive group on the lawn or support reacting with the building block.

Artificial Receptors

The present invention relates to artificial receptors and compositions that can form such receptors, e.g., candidate artificial receptors. The artificial receptors or compositions include building blocks reversibly immobilized on a support. The building blocks can be reversibly immobilized through any of a variety of interactions, such as covalent, ionic, or hydrophobic interactions.

In an embodiment, the composition includes molecules forming a lawn and coupled to the support. The building blocks can be reversibly immobilized through interactions with the lawn. In an embodiment, the present composition includes a support, a functionalized lawn, and a plurality of building blocks. The functionalized lawn can be coupled to the support. The plurality of building blocks can be reversibly immobilized on the lawn.

The building blocks can be reversibly immobilized on the lawn or support through, for example, readily reversible covalent bonding or noncovalent interactions. For such interactions, the lawn or support includes a functional group or moiety suitable for forming a readily reversible covalent bond or noncovalent interaction with the building block. Similarly, the building block includes a functional group or moiety suitable for forming a readily reversible covalent bond or noncovalent interaction with the lawn or support. For example, the building block and support or lawn each include one or more functional groups or moieties that can form readily reversible covalent, ionic, hydrogen bonding, van der Waals, or like interactions.

In an embodiment, the support includes a surface or region functionalized to include moieties suitable for a reversible interaction with the building block. In an embodiment, the support includes moieties that can engage in reversible covalent bonding or noncovalent interactions.

In an embodiment, the support includes moieties that can engage in reversible covalent bonding. Suitable groups for reversible covalent bonding are described hereinabove. A composition, such as a candidate artificial receptor, can include building blocks reversibly immobilized on the support through, for example, imine, acetal, ketal, disulfide, ester, and like linkages. An artificial receptor can include functional groups on the support that are not linked to a building block and support functional groups covalently linked to a building block.

In an embodiment, the support includes moieties that can engage in noncovalent interactions. For example, the support can include functional groups such as an ionic group, a group that can hydrogen bond, or a group that can engage in van der Waals or other hydrophobic interactions. A composition, such as a candidate artificial receptor, can include building blocks reversibly immobilized on the support through ionic interactions. An artificial receptor can include both free ionic groups on the support and support ionic groups ionically linked to a building block. A composition, such as a candidate artificial receptor, can include building blocks reversibly immobilized on the support through hydrogen bonding. An artificial receptor can include both free hydrogen bonding groups on the support and support hydrogen bonding groups hydrogen bonded to a building block. A composition, such as a candidate artificial receptor, can include building blocks reversibly immobilized on the support through hydrophobic interactions. An artificial receptor can include both free hydrophobic groups on the support and support hydrophobic groups interacting with a building block.

In an embodiment, the support includes ionic groups, such as cationic groups, anionic groups, or mixtures thereof. The support can include a surface or region with ionic groups. For example, the support can include a surface or region including one or more cationic groups (e.g., at neutral pH in aqueous compositions) such as amines, quaternary ammonium moieties, sulfonium, phosphonium, ferrocene, or the like. For example, the support can include a surface or region including one or more anionic groups (e.g., at neutral pH in aqueous compositions) such as carboxylates, phenols substituted with strongly electron withdrawing groups (e.g., tetrachlorophenols), phosphates, phosphonates, phosphinates, sulphates, sulphonates, thiocarboxylates, hydroxamic acids, or the like. In an embodiment, the charge on the group relates to the charge at neutral pH in aqueous compositions.

In an embodiment, the support includes groups that can hydrogen bond, either as donors or acceptors. The support can include a surface or region with groups that can hydrogen bond. Suitable groups for hydrogen bonding include those described hereinabove. Ionic groups can also participate in hydrogen bonding.

In an embodiment, the support includes a hydrophobic or lipophilic group. The support can include a surface or region with hydrophobic or lipophilic groups. For example, the support can include a surface or region including one or more of the hydrophobic or lipophilic groups described hereinabove.

In an embodiment, the composition or artificial receptor includes a lawn (e.g., a functionalized lawn) coupled to a surface or region on the support. The lawn can be coupled to the support through covalent bonds that are stable under a variety of conditions such that it is difficult to remove the lawn from the support. For example, in an embodiment, the lawn cannot be uncoupled from the support under conditions that cleave a readily reversible covalent bond. The lawn reagent can include any of a variety of functional groups that can be coupled to the support plus any of a variety of functional groups that can reversibly interact with the building block. For example, the lawn can include one or more moieties that can engage in reversible covalent bonding or noncovalent interactions with the building block.

In an embodiment, the lawn includes moieties that can engage in reversible covalent bonding. Suitable functional groups for reversible covalent bonding are described hereinabove. An artificial receptor can include building blocks reversibly immobilized on the lawn through imine, acetal, ketal, disulfide, ester, or like linkages. An artificial receptor can include both free functional groups on the lawn and lawn functional groups covalently linked to a building block.

In an embodiment, the lawn includes moieties that can engage in noncovalent interactions. For example, the lawn can include functional groups such as an ionic group, a group that can hydrogen bond, or a group that can engage in van der Waals or other hydrophobic interactions. An artificial receptor can include building blocks reversibly immobilized on the lawn through ionic interactions. Suitable functional groups for ionic interactions are described hereinabove. An artificial receptor can include both free ionic groups on the lawn and lawn ionic groups ionically linked to a building block.

An artificial receptor can include building blocks reversibly immobilized on the lawn through hydrogen bonding. Suitable functional groups for hydrogen bonding interactions are described hereinabove. An artificial receptor can include both free hydrogen bonding groups on the lawn and lawn hydrogen bonding groups hydrogen bonded to a building block.

An artificial receptor can include building blocks reversibly immobilized on the lawn through hydrophobic interactions. Suitable functional groups for hydrophobic interactions are described hereinabove. An artificial receptor can include both free hydrophobic groups on the lawn and lawn hydrophobic groups interacting with a building block.

In an embodiment the present methods and compositions can include building blocks that are coupled to the support in a manner that is essentially irreversible. For example, an irreversibly coupled building block can be coupled through a covalent bond that cannot be broken without damaging the artificial receptor. In an embodiment, irreversible coupling employs a covalent bond that is stable under conditions used to reverse the reversible covalent bond. In an embodiment, an amide bond irreversibly couples a building block to a support. According to the present invention, an artificial receptor including n building blocks can include as many as n−1 irreversibly immobilized building blocks and 1 reversibly immobilized building block.

Illustrated Embodiments of Artificial Receptors

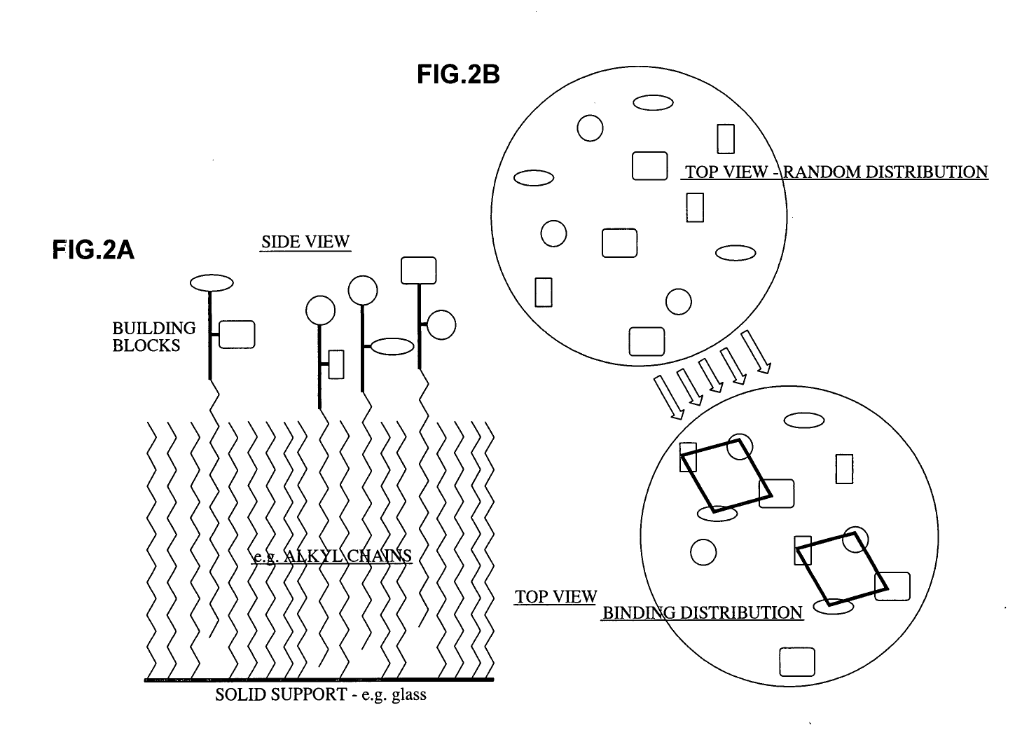

FIG. 2A schematically illustrates an embodiment of an artificial receptor including building blocks reversibly immobilized through hydrophobic interactions with a lawn on a solid support. In this embodiment, the hydrophobic interactions are provided by long unbranched alkyl chains. Building blocks can be synthesized with long chain alkyl or alkyl-like linkers appended to the framework through, e.g., a carboxyl moiety. The support can include an amino surface modified by reaction with, e.g., activated long chain fatty acids to form an alkyl (or alkyl-like) lawn. Addition of the building blocks to the surface environment leads to incorporation of at least some of the building blocks into the lawn with the portion of the building block including the recognition elements (e.g., ligand binding portion) on the surface of the lawn. The surface of the artificial receptor can also include any of a variety of solvent environments.

FIG. 2B schematically illustrates that the building blocks can achieve a random distribution on a region of the support and rearrange. Upon exposure to a test ligand, mobilized building blocks can rearrange to provide improved binding of the test ligand. Although not limiting the present invention, this binding and rearrangement can be envisioned as initial binding of a test ligand followed by kinetically and/or thermodynamically driven spatial redistribution of the building blocks. Such spatial redistribution can improve or optimize interactions between the artificial receptor and the test ligand. Such kinetic or thermodynamic improvement or optimization can be viewed as “evolution” toward greater binding affinity in an environment that can have mobile and/or immobilized building blocks.

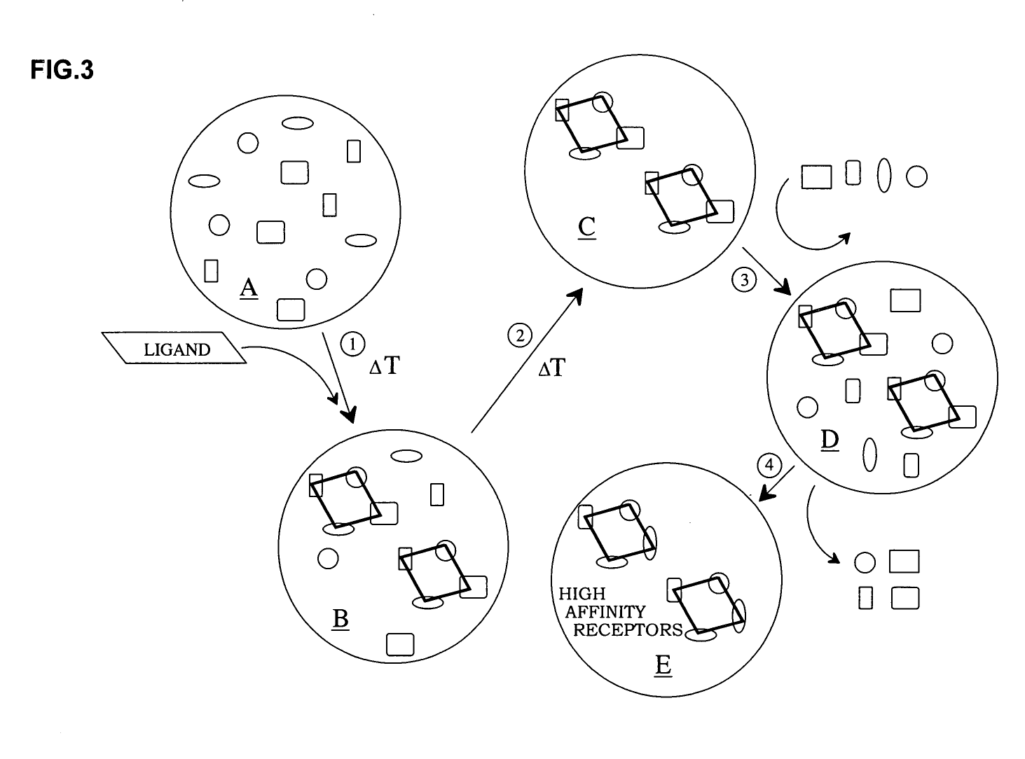

FIG. 3 schematically illustrates an embodiment employing the present artificial receptors to develop a lead artificial receptor using shuffling and exchanging of building blocks. View A of the artificial receptor schematically illustrates the building blocks in a random distribution on a region of the support. The building blocks and lawn can include, for example, the alkyl tails schematically illustrated in FIG. 2A.

Reaction 1 includes contacting the artificial receptor with a test ligand. Reaction 1 as illustrated also includes a change in temperature to allow building blocks to shuffle or rearrange within the receptor, which can improve binding to the test ligand. In another embodiment, shuffling or rearranging can be induced by other changes in conditions, such as change in solvent composition or a combination of change in temperature and solvent. View B of the artificial receptor schematically illustrates the rearranged building blocks with bound test ligand and also building blocks not bound to the test ligand.

Reaction 2 further mobilizes the building blocks and allows unbound building blocks to exchange off of the artificial receptor surface. Reaction 2 as illustrated also includes a change in temperature sufficient to allow building blocks to exchange into fluid contacting the artificial receptor. Such a temperature change may be larger than the temperature change in reaction 1. In another embodiment, this exchange can be accomplished, for example, by a change in conditions such as changing solvent composition (e.g., contacting with a more hydrophobic solvent), by increasing temperature and changing solvent composition, or the like. The change in conditions used to achieve exchange can be larger or more pronounced than a change used to achieve shuffling. Although not limiting to the present invention, this exchange can be viewed as increasing the on/off rate of the building blocks and leading to loss of the building blocks which are not protected by interaction with the target. View C of the artificial receptor schematically illustrates the rearranged building blocks with bound test ligand and the absence of building blocks exchanged off of the artificial receptor.

Reaction 3 exchanges additional building blocks onto the artificial receptor. Reaction 3 can include changing the conditions as described for exchanging building blocks off of the artificial receptor. View D schematically illustrates the artificial receptor including the added building blocks. Although not limiting to the present invention, the reaction can be considered affinity maturation of a receptor, exchanging one or more of the first set of building blocks for one or more building blocks which may have higher affinity for the test ligand.

Reaction 4, similar to reactions 1 and 2, shuffles or rearranges building blocks within the receptor and exchanges unbound building blocks off of the artificial receptor. This reaction can use conditions as described for reactions 1 and 2. View E schematically illustrates the artificial receptor with shuffled and exchanged building blocks bound to the test ligand. In an embodiment, the artificial receptor with the added building blocks has greater affinity for the test ligand than did the preceding receptor-ligand complexes. Although not limiting to the present invention, this process can be considered as equilibrium driven affinity maturation.

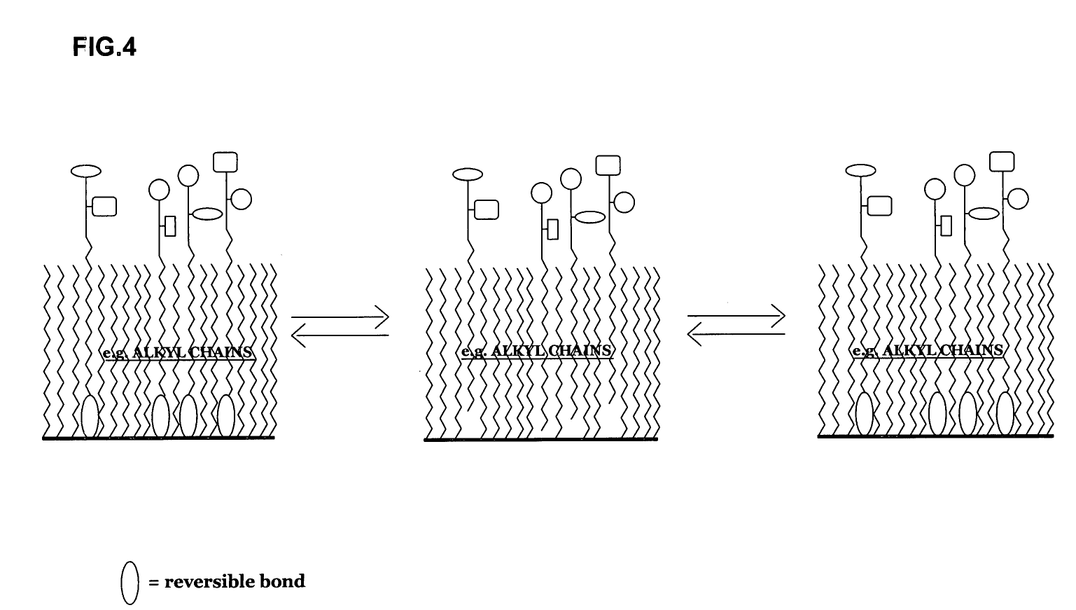

FIG. 4 schematically illustrates an embodiment of the artificial receptor shown in FIG. 2A. This embodiment includes building blocks reversibly immobilized through hydrophobic interactions with a lawn on a solid support. The hydrophobic interactions are provided by long alkyl chains. The hydrophobic interactions by themselves can be sufficient to reversibly immobilize the building block. In addition, the lawn or support and the alkyl chain on the building block each include a functional group or moiety that can form a reversible covalent bond. Forming the covalent bond can fix the building block in a particular location on the support of the artificial receptor. The building block can, for example, remain fixed under conditions suitable to mobilize building blocks reversibly immobilized only by hydrophobic interactions. Such as system can provide selective mobility of some but not all building blocks. Breaking the covalent bond can allow the building block mobility within the hydrophobic environment of the artificial receptor (e.g., to translate or shuffle) and to be released from the support and hydrophobic environment (e.g., to exchange).

In this embodiment, the receptor can begin either with the building block fixed by the covalent bond, fixed by the hydrophobic interaction, or both. For example, the building blocks can be initially fixed in position by the reversible covalent bond. Breaking the reversible covalent bond can allow mobility of the building block. Mobilization can allow affinity optimization or improvement of the artificial receptor. Although not limiting to the present invention, this approach can allow greater initial time for kinetic and thermodynamic equilibration of interactions between the test ligand and the artificial receptor before the onset of more stringent conditions. By way of further example, the building blocks can initially be reversibly immobilized on or in a place on the lawn by hydrophobic interactions and then be fixed into position by a covalent bond after binding of a test ligand. Although not limiting to the present invention, this approach can allow fixing the artificial receptors in a configuration useful for or optimal for binding test ligand, which can increase stability of the receptor:ligand complex.

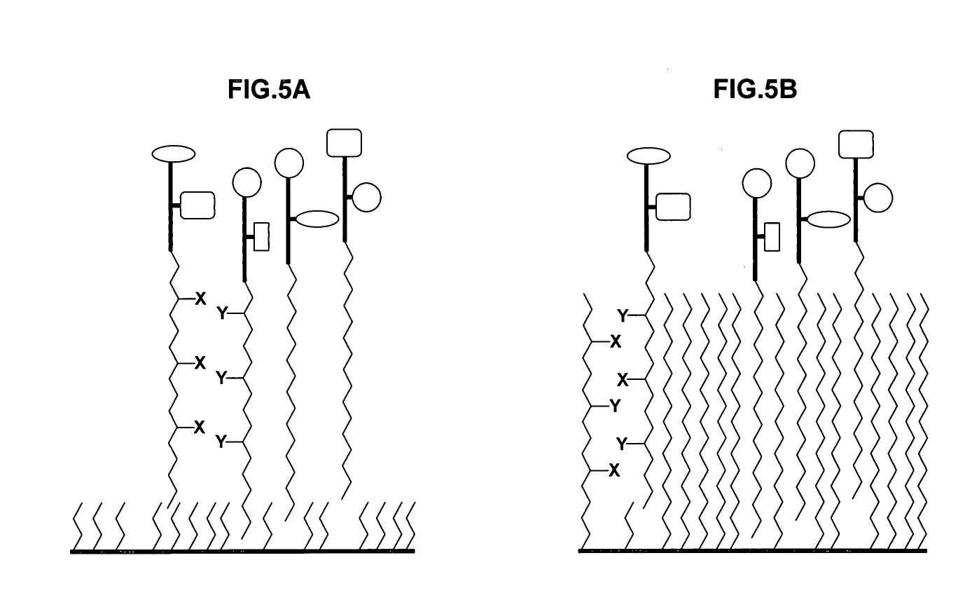

FIGS. 5A and 5B schematically illustrate embodiments of the artificial receptor shown in FIG. 2A. These embodiments include building blocks reversibly immobilized through hydrophobic interactions with a lawn on a solid support. The hydrophobic interactions are provided by alkyl chains on the support and/or building block. The lawn and/or the alkyl chain on the building block can each include one or more functional groups or moieties that can form a reversible bond, such as a reversible covalent bond, an ionic interaction, or a hydrogen bond. FIG. 5A illustrates an embodiment in which building blocks can be reversibly bound one to the other. FIG. 5B illustrates an embodiment in which one or more building blocks can be reversibly bound to one or more molecules making up the lawn. In the embodiments illustrated in FIGS. 5A and 5B, reversible bonds between the alkyl chains can control the position and/or mobility of the building blocks during or after binding of a test ligand. The various types of reversible immobilization of the present invention can provide variable degrees of building block mobility on the support.

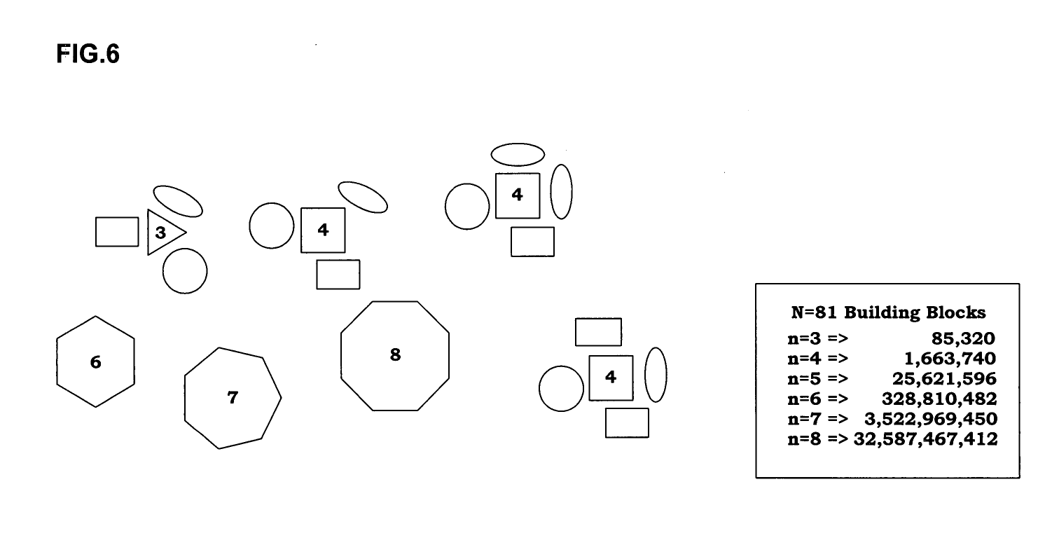

Referring now to FIG. 6, in an embodiment, a strategy employing the present artificial receptors with reversibly immobilized building blocks can provide convenient access to millions and even billions of different artificial receptors. Starting with, for example, 81 different building blocks, combinations of 2, 3, 4, 5, or more building blocks quickly yield more than several million artificial receptors including more than one building block. For example, 81 building blocks provide 85,320 combinations of three building blocks and 1,663,740 combinations of four building blocks. If an artificial receptor is a spot in a microarray, with 100,000 spots on a slide, the number of slides to contain millions of combinations of building blocks can become unwieldy. Reversible immobilization of building blocks can provide convenient access to several-fold more artificial receptors.

FIG. 6 schematically illustrates test ligands with 3, 4, 5, 6, 7, or 8 binding surfaces or environments as polygons with 3, 4, 5, 6, 7, or 8 sides. For small molecules, the number of surfaces or environments may be limited, for example, to 2, 3, or 4. However, for macromolecules the number of surfaces or environments can be significantly larger, for example, 6, 7, or 8. The present invention, through shuffling and exchanging reversibly immobilized building blocks can allow access to large number of combinations of up to, for example, 8 building blocks in an artificial receptor. Such a process can begin with a convenient number of initial receptors, which can be tested for binding of a test ligand. These artificial receptors can than undergo exchange of additional building blocks until the receptors include up to 8 building blocks. For a set of 81 building blocks, being able to test combinations of 8 building blocks through exchange and shuffling can give practical access to 32 billion artificial receptors (the number of combinations of 8 from a set of 81), without making 32 billion spots in an array.

Embodiments of Artificial Receptors

In an embodiment, the present artificial receptors and methods provide an initial binding event that produces a lead artificial receptor. This lead artificial receptor can then be improved through both shuffling and exchange of receptor substructures. Such compositions and methods employ combinatorial presentation of a large number of receptor building blocks for probing to find a lead artificial receptor. Then, these compositions and methods allow dynamic, spatial redistribution of building blocks for improving binding by the lead artificial receptor.

In an embodiment, reversible mobilization of building blocks on a support provides cooperative interaction of the building blocks with one another and/or with the ligand. This can favor interactive molecular recognition. By way of contrast, conventional dynamic combinatorial libraries (DCL) employ ligand and receptor subunits free in bulk solution. With all components free in bulk solution, each receptor subunit is only held in coordination with the ligand by the weak interactions between the individual subunits and the ligand. In DCL, improvement in binding is limited by dissociation of the building block into the surrounding solution. Thus, the present invention including reversible immobilization of building blocks on a surface provides significant advantages over conventional, solution based DCL.

In an embodiment, cooperative interaction of building blocks and ligand can be envisioned as follows. The ligand can be bound to n building blocks of an artificial receptor. Shuffling can be employed to induce 1 to n−1 of the building blocks to move on the receptor to a different or improved position for binding the ligand or to shuffle away from the ligand. In an embodiment, the ligand can also move and remain bound to one or more building blocks on the artificial receptor surface. In this manner, the cooperative interaction of building block and ligand can alter or improve ligand binding without the ligand being released from the artificial receptor.

In an embodiment, the artificial receptor includes a candidate artificial receptor, a lead artificial receptor, a working artificial receptor, or a combination thereof. One or more lead artificial receptors can be developed from a plurality of candidate artificial receptors. In an embodiment, a lead artificial receptor includes a combination of building blocks and binds detectable quantities of test ligand upon exposure to, for example, several picomoles of test ligand at a concentration of 1, 0.1, or 0.01 μg/ml, or at 1, 0.1, or 0.01 ng/ml test ligand; at a concentration of 0.01 μg/ml, or at 1, 0.1, or 0.01 ng/ml test ligand; or a concentration of 1, 0.1, or 0.01 ng/ml test ligand.

One or more working artificial receptors can be developed from one or more lead artificial receptors. In an embodiment, a working artificial receptor includes a combination of building blocks and binds categorizing or identifying quantities of test ligand upon exposure to, for example, several picomoles of test ligand at a concentration of 100, 10, 1, 0.1, 0.01, or 0.001 ng/ml test ligand; at a concentration of 10, 1, 0.1, 0.01, or 0.001 ng/ml test ligand; or a concentration of 1, 0.1, 0.01, or 0.001 ng/ml test ligand.

Building Blocks

The present invention relates to building blocks for making or forming candidate artificial receptors. Building blocks are designed, made, and selected to provide a variety of structural characteristics among a small number of compounds. The present building blocks also include a functional group or structural feature or moiety that allows them to be reversibly immobilized on a support, e.g., by way of a lawn.

A building block can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A building block can be bulky or it can be small.

A building block can be visualized as including several components, such as one or more frameworks, one or more linkers, and/or one or more recognition elements. The framework can be covalently coupled to each of the other building block components. The recognition element can be covalently coupled to the framework. The linker can be covalently coupled to the framework and reversibly coupled to a support or to a lawn molecule. In an embodiment, a building block includes a framework, a linker, and a recognition element. In an embodiment, a building block includes a framework, a linker, and two recognition elements.

A description of general and specific features and functions of a variety of building blocks and their synthesis can be found in copending U.S. patent application Ser. No. 10/244,727, filed Sep. 16, 2002, and Application No. PCT/US03/05328, filed Feb. 19, 2003, each entitled “ARTIFICIAL RECEPTORS, BUILDING BLOCKS, AND METHODS”, and U.S. Provisional Patent Application Serial No.______, also entitled “ARTIFICIAL RECEPTORS, BUILDING BLOCKS, AND METHODS”, filed evendate herewith, the disclosures of which are incorporated herein by reference. These patent documents include, in particular, a detailed written description of: function, structure, and configuration of building blocks, framework moieties, recognition elements, synthesis of building blocks, specific embodiments of building blocks, specific embodiments of recognition elements, and sets of building blocks.

Framework

The framework can be selected for functional groups that provide for coupling to the recognition moiety and for coupling to or being the linking moiety. The framework can interact with the ligand as part of the artificial receptor. In an embodiment, the framework includes multiple reaction sites with orthogonal and reliable functional groups and with controlled stereochemistry. Suitable functional groups with orthogonal and reliable chemistries include, for example, carboxyl, amine, hydroxyl, phenol, carbonyl, and thiol groups, which can be individually protected, deprotected, and derivatized. In certain embodiments, the framework has two, three, or four functional groups with orthogonal and reliable chemistries.

The three functional groups can be independently selected, for example, from carboxyl, amine, hydroxyl, phenol, carbonyl, or thiol group. The framework can include alkyl, substituted alkyl, cycloalkyl, heterocyclic, substituted heterocyclic, aryl alkyl, aryl, heteroaryl, heteroaryl alkyl, and like moieties.

The functional groups can be appended to an organic moiety, R₁. R₁ can be a 1-12, 1-6, or 1-4 carbon alkyl, substituted alkyl, cycloalkyl, heterocyclic, substituted heterocyclic, aryl alkyl, aryl, heteroaryl, heteroaryl alkyl, or like group; and the functional groups can independently be a carboxyl, amine, hydroxyl, phenol, carbonyl, or thiol group. The functional groups can independently be a 1-12, 1-6, or 1-4 carbon alkyl, substituted alkyl, cycloalkyl, heterocyclic, substituted heterocyclic, aryl alkyl, aryl, heteroaryl, heteroaryl alkyl, or inorganic group substituted with carboxyl, amine, hydroxyl, phenol, carbonyl, or thiol group. The framework can include 2, 3, 4 or more functional groups.

A variety of compounds fit the schemes and formulas describing the framework including amino acids, and naturally occurring or synthetic compounds including, for example, oxygen and sulfur functional groups. The compounds can be racemic, optically active, or achiral. For example, the compounds can be natural or synthetic amino acids, α-hydroxy acids, thioic acids, and the like.

Suitable molecules for use as a framework include a natural or synthetic amino acid, particularly an amino acid with a functional group (e.g., third functional group) on its side chain. Amino acids include carboxyl and amine functional groups. The side chain functional group can include, for natural amino acids, an amine (e.g., alkyl amine, heteroaryl amine), hydroxyl, phenol, carboxyl, thiol, thioether, or amidino group. Natural amino acids suitable for use as frameworks include, for example, serine, threonine, tyrosine, aspartic acid, glutamic acid, asparagine, glutamine, cysteine, lysine, arginine, histidine. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Suitable synthetic amino acids include β-amino acids and homo or β analogs of natural amino acids.

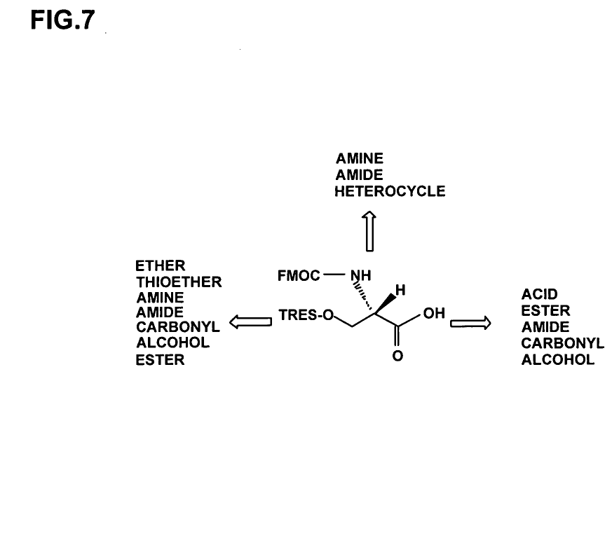

Suitable framework amino acids include serine, threonine, or tyrosine, e.g., serine or tyrosine, e.g., tyrosine. FIG. 7 illustrates serine as a framework for a building block and reactions for forming building blocks from serine, tyrosine, and other amino acids. Threonine and tyrosine can exhibit reactivity similar to serine.

All of the naturally occurring and many synthetic amino acids are commercially available. Further, forms of these amino acids derivatized or protected to be suitable for reactions for coupling to recognition element(s) and/or linkers can be purchased or made by known methods (see, e.g., Green, TW; Wuts, PGM (1999), Protective Groups in Organic Synthesis Third Edition, Wiley-Interscience, New York, 779 pp.; Bodanszky, M.; Bodanszky, A. (1994), The Practice of Peptide Synthesis Second Edition, Springer-Verlag, New York, 217 pp.).

Recognition Element

The recognition element can be selected to provide one or more structural characteristics to the building block. The framework and/or recognition element can interact with the ligand as part of the artificial receptor. For example, the recognition element can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A recognition element can be a small group or it can be bulky.

In an embodiment, the recognition element can be a 1-12, 1-6, or 1-4 carbon alkyl, substituted alkyl, cycloalkyl, heterocyclic, substituted heterocyclic, aryl alkyl, aryl, heteroaryl, heteroaryl alkyl, or like group. The recognition element can be substituted with a group that includes or imparts positive charge, negative charge, acid, base, electron acceptor, electron donor, hydrogen bond donor, hydrogen bond acceptor, free electron pair, π electrons, charge polarization, hydrophilicity, hydrophobicity, and the like.

Recognition elements with a positive charge (e.g., at neutral pH in aqueous compositions) include amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, e.g., 1-8 carbons, rings can have 3-12 carbons, e.g., 3-8 carbons. Suitable alkyl amines include that of formula B9. Suitable heterocyclic or alkyl heterocyclic amines include that of formula A9. Suitable pyridines include those of formulas A5 and B5. Any of the amines can be employed as a quaternary ammonium compound. Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like.

Recognition elements with a negative charge (e.g., at neutral pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

Recognition elements with a negative charge and a positive charge (at neutral pH in aqueous compositions) include sulfoxides, betaines, and amine oxides.

Acidic recognition elements can include carboxylates, phosphates, sulphates, and phenols. Suitable acidic carboxylates include thiocarboxylates. Suitable acidic phosphates include the phosphates listed hereinabove.

Basic recognition elements include amines. Suitable basic amines include alkyl amines, aryl amines, aryl alkyl amines, pyridines, heterocyclic amines (saturated or unsaturated, the nitrogen in the ring or not), amidines, and any additional amines listed hereinabove. Suitable alkyl amines include that of formula B9. Suitable heterocyclic or alkyl heterocyclic amines include that of formula A9. Suitable pyridines include those of formulas A5 and B5.

Recognition elements including a hydrogen bond donor include amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable alkyl amines include that of formula B9. Suitable heterocyclic or alkyl heterocyclic amines include that of formula A9. Suitable pyridines include those of formulas A5 and B5. Suitable protonated carboxylates, protonated phosphates include those listed hereinabove. Suitable amides include those of formulas A8 and B8. Suitable alcohols include primary alcohols, secondary alcohols, tertiary alcohols, and aromatic alcohols (e.g., phenols). Suitable alcohols include those of formulas A7 (a primary alcohol) and B7 (a secondary alcohol).

Recognition elements including a hydrogen bond acceptor or one or more free electron pairs include amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable alkyl amines include that of formula B9. Suitable heterocyclic or alkyl heterocyclic amines include that of formula A9. Suitable pyridines include those of formulas A5 and B5. Suitable carboxylates include those listed hereinabove. Suitable amides include those of formulas A8 and B8. Suitable phosphates, phosphonates and phosphinates include those listed hereinabove. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include alkyl ethers, aryl alkyl ethers. Suitable alkyl ethers include that of formula A6. Suitable aryl alkyl ethers include that of formula A4. Suitable thioethers include that of formula B6.

Recognition elements including uncharged polar or hydrophilic groups include amides, alcohols, ethers, thiols, thioethers, esters, thio esters, boranes, borates, and metal complexes. Suitable amides include those of formulas A8 and B8. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include those listed hereinabove. Suitable ethers include that of formula A6. Suitable aryl alkyl ethers include that of formula A4.

Recognition elements including uncharged hydrophobic groups include alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable lower alkyl groups include those of formulas A1, A3, A3a, and B1. Suitable aryl alkyl groups include those of formulas A3, A3a, A4, B3, B3a, and B4. Suitable alkyl cycloalkyl groups include that of formula B2. Suitable alkene groups include lower alkene and aryl alkene. Suitable aryl alkene groups include that of formula B4. Suitable aromatic groups include unsubstituted aryl, heteroaryl, substituted aryl, aryl alkyl, heteroaryl alkyl, alkyl substituted aryl, and polyaromatic hydrocarbons. Suitable aryl alkyl groups include those of formulas A3, A3a and B4. Suitable alkyl heteroaryl groups include those of formulas A5 and B5.

Spacer (e.g., small) recognition elements include hydrogen, methyl, ethyl, and the like. Bulky recognition elements include 7 or more carbon or hetero atoms.

Formulas A1-A9 and B1-B9 are:

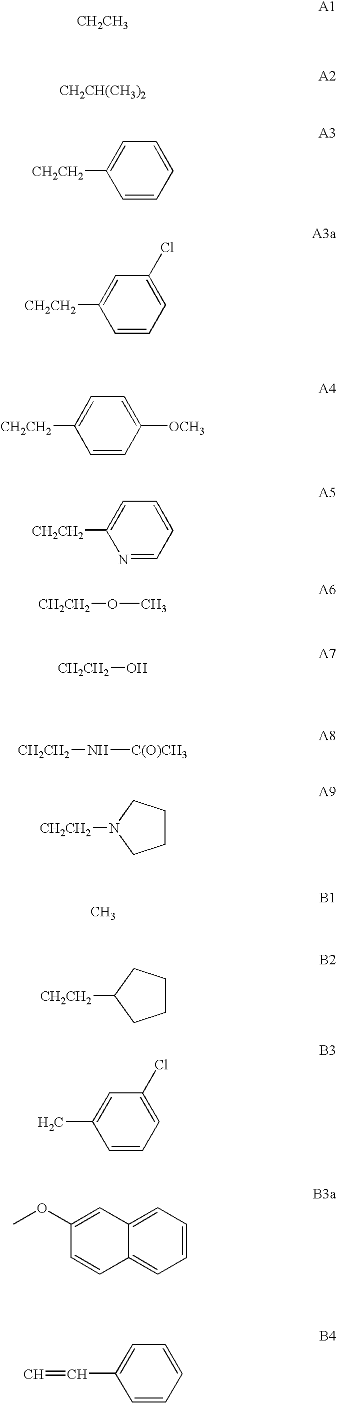

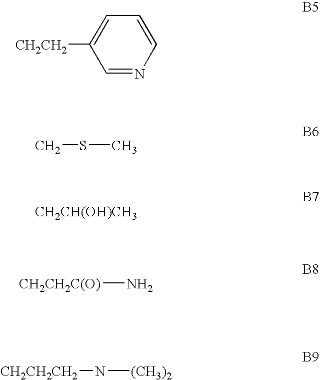

These A and B recognition elements can be called derivatives of, according to a standard reference: A1, ethylamine; A2, isobutylamine; A3, phenethylamine; A4, 4-methoxyphenethylamine; A5,2-(2-aminoethyl)pyridine; A6,2-methoxyethylamine; A7, ethanolamine; A8, N-acetylethylenediamine; A9,1-(2-aminoethyl)pyrrolidine; B 1, acetic acid, B2, cyclopentylpropionic acid; B3,3-chlorophenylacetic acid; B4, cinnamic acid; B5, 3-pyridinepropionic acid; B6, (methylthio)acetic acid; B7,3-hydroxybutyric acid; B8, succinamic acid; and B9,4-(dimethylamino)butyric acid.

In an embodiment, the recognition elements include one or more of the structures represented by formulas A1, A2, A3, A3a, A4, A5, A6, A7, A8, and/or A9 (the A recognition elements) and/or B1, B2, B3, B3a, B4, B5, B6, B7, B8, and/or B9 (the B recognition elements). In an embodiment, each building block includes an A recognition element and a B recognition element. In an embodiment, a group of 81 such building blocks includes each of the 81 unique combinations of an A recognition element and a B recognition element. In an embodiment, the A recognition elements are linked to a framework at a pendant position. In an embodiment, the B recognition elements are linked to a framework at an equatorial position. In an embodiment, the A recognition elements are linked to a framework at a pendant position and the B recognition elements are linked to the framework at an equatorial position.

In an embodiment, the building blocks including the A and B recognition elements can be visualized as occupying a binding space defined by lipophilicity/hydrophilicity and volume. A volume can be calculated (using known methods) for each building block including the various A and B recognition elements. A measure of lipophilicity/hydrophilicity (logP) can be calculated (using known methods) for each building block including the various A and B recognition elements. Negative values of logP show affinity for water over nonpolar organic solvent and indicate a hydrophilic nature. A plot of volume versus logP can then show the distribution of the building blocks through a binding space defined by size and lipophilicity/hydrophilicity.

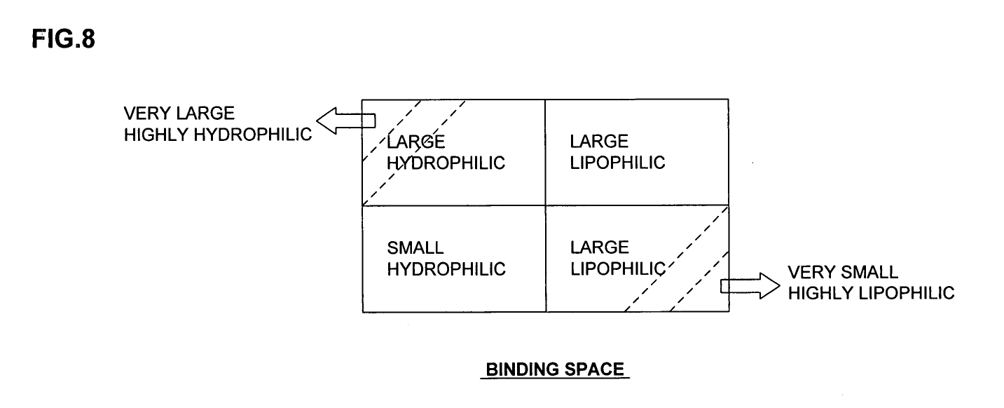

FIG. 8 schematically illustrates binding space divided qualitatively into 4 quadrants—large hydrophilic, large hydrophobic, small hydrophilic, and small lipophilic. FIG. 8 denotes a small triangle of the large hydrophilic quadrant as very large and highly hydrophilic. FIG. 8 denotes a small triangle of the small lipophilic quadrant as very small and highly lipophilic.

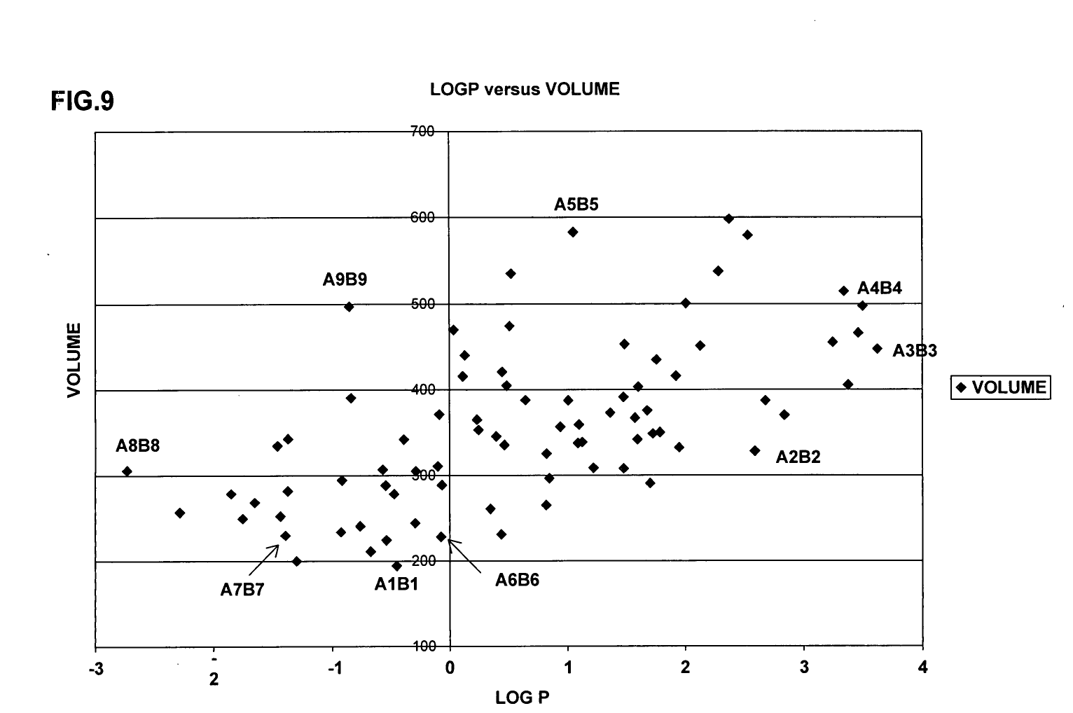

FIG. 9 illustrates a plot of volume versus logP for 81 building blocks including each of the 9A and 9B recognition elements. This plot illustrates that the 81 building blocks with A and B recognition elements fill a significant portion of the binding space defined by volume and lipophilicity/hydrophilicity. The space filled by the 81 building blocks is roughly bounded by the A1B1, A2B2, . . . A9B9 building blocks (FIG. 9). The 81 building blocks with A and B recognition elements fill a majority of this binding space excluding only the portion denoted very large and highly hydrophilic and the portion denoted very small and highly lipophilic.

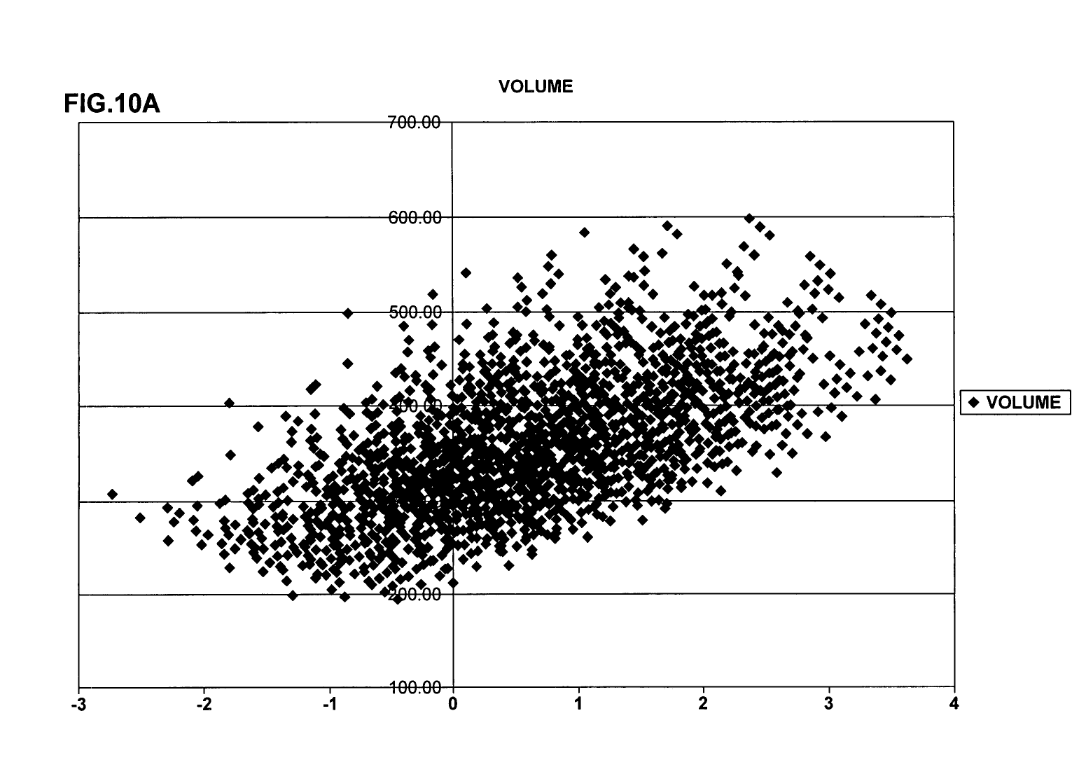

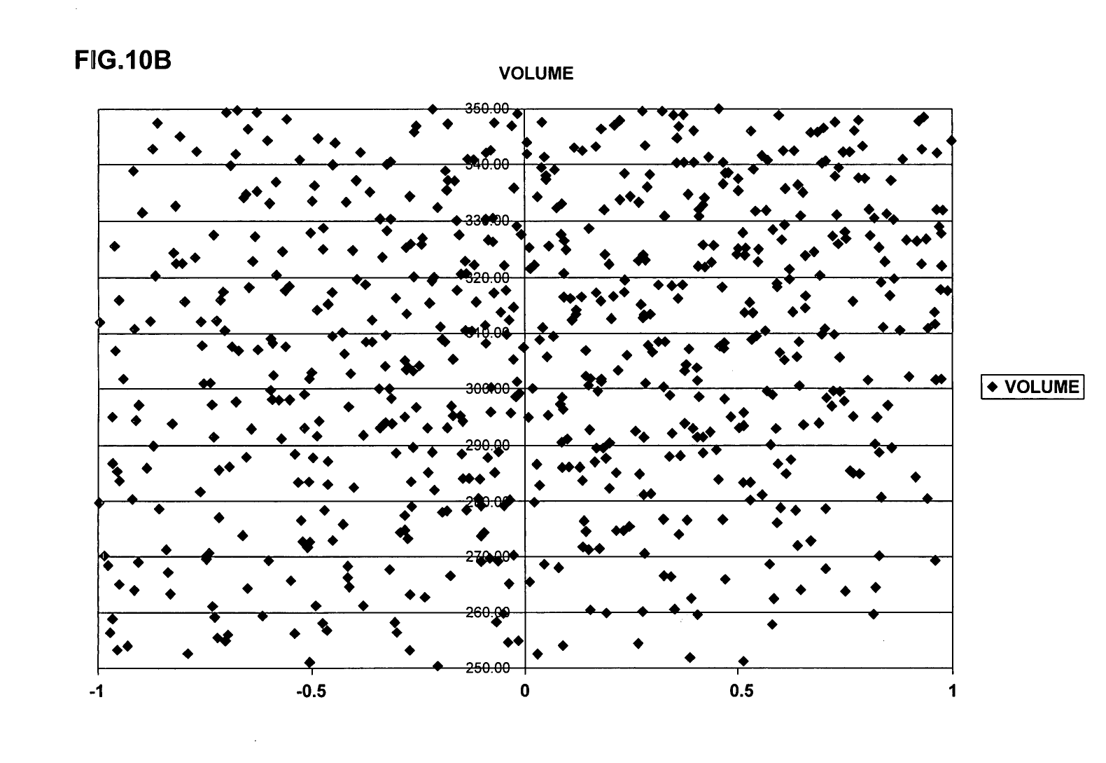

FIGS. 10A and 10B illustrate a plot of volume versus logP for combinations of building blocks with A and B recognition elements forming candidate artificial receptors. The volumes and values of logP for these candidate artificial receptors generally fill in the space occupied by the individual building blocks. FIG. 10B represents a detail from FIG. 10A. This detail illustrates that the candidate artificial receptors fill the binding space evenly. Candidate artificial receptors made from building blocks with A and B recognition elements include receptors with a wide range of sizes and a wide range of values of lipophilicity/hydrophilicity.

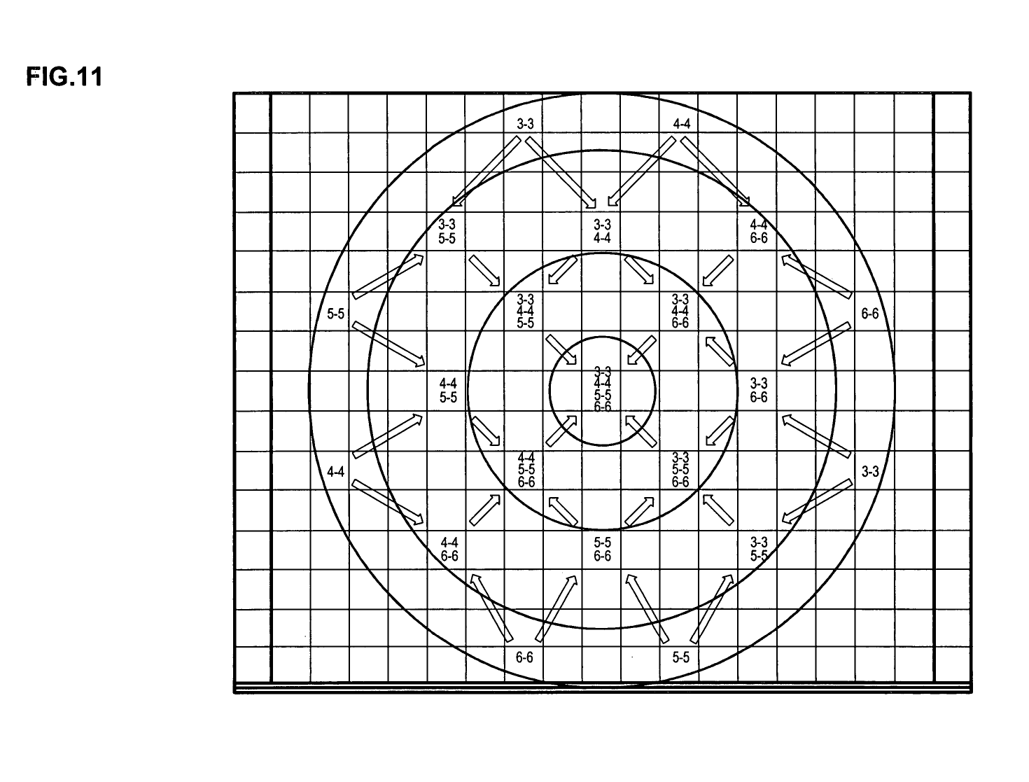

FIG. 11 illustrates that candidate artificial receptors made up of building blocks can be sorted and evaluated with respect to their nearest neighbors, other candidate artificial receptors made up of one or more of the same building blocks. In an embodiment, the nearest neighbor can be made up of a subset of the building blocks forming the subject candidate artificial receptor. For example, as shown in FIG. 11, a candidate artificial receptor made up of TyrA3B3/TyrA4B4/TyrA5B5/TyrA6B6 has among its nearest neighbors candidate artificial receptors TyrA4B4/TyrA5B5/TyrA6B6, TyrA3B3/TyrA5B5/TyrA6B6, TyrA3B3/TyrA4B4/TyrA6B6, and TyrA3B3/TyrA4B4/TyrA5B5. These candidate artificial receptors in turn have additional nearest neighbors. Candidate receptors and/or recognition elements can also be grouped as neighbors based on lipophilicity/hydrophilicity, size, charge, or another physical or chemical characteristic.

Reagents that form many of the recognition elements are commercially available. For example, reagents for forming recognition elements A1, A2, A3, A3a, A4, A5, A6, A7, A8, A9 B1, B2, B3, B3a, B4, B5, B6, B7, B8, and B9 are commercially available.

Linkers

The linker is selected to provide suitable reversible immobilization of the building block on a support or lawn. The linker can interact with the ligand as part of the artificial receptor. The linker can also provide bulk, distance from the support, hydrophobicity, hydrophilicity, and like structural characteristics to the building block. In an embodiment, the linker forms a covalent bond with a functional group on the framework. In an embodiment, the linker also includes a functional group that can reversibly interact with the support or lawn, e.g., through reversible covalent bonding or noncovalent interactions.

In an embodiment, the linker includes one or more moieties that can engage in reversible covalent bonding. Suitable groups for reversible covalent bonding include those described hereinabove. An artificial receptor can include building blocks reversibly immobilized on the lawn or support through, for example, imine, acetal, ketal, disulfide, ester, or like linkages. Such functional groups can engage in reversible covalent bonding. Such a functional group can be referred to as a covalent bonding moiety, e.g., a second covalent bonding moiety.

In an embodiment, the linker can be functionalized with moieties that can engage in noncovalent interactions. For example, the linker can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the present methods and compositions can employ a linker including a charged moiety (e.g., a second charged moiety). Suitable charged moieties include positively charged moieties and negatively charged moieties. Suitable positively charged moieties include amines, quaternary ammonium moieties, sulfonium, phosphonium, ferrocene, and the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, and hydroxamic acids.

In an embodiment, the present methods and compositions can employ a linker including a group that can hydrogen bond, either as donor or acceptor (e.g., a second hydrogen bonding group). For example, the linker can include one or more carboxyl groups, amine groups, hydroxyl groups, carbonyl groups, or the like. Ionic groups can also participate in hydrogen bonding.

In an embodiment, the present methods and compositions can employ a linker including a lipophilic moiety (e.g., a second lipophilic moiety). Suitable lipophilic moieties include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. In an embodiment the linker includes or is a lipid, such as a phospholipid.

In an embodiment, the linker includes a lipophilic moiety (e.g., a second lipophilic moiety) and a covalent bonding moiety (e.g., a second covalent bonding moiety). In an embodiment, the linker includes a lipophilic moiety (e.g., a second lipophilic moiety) and a charged moiety (e.g., a second charged moiety).

In an embodiment, the linker can form or can be visualized as forming a covalent bond with an alcohol, phenol, thiol, amine, carbonyl, or like group on the framework. Between the bond to the framework and the group participating in or formed by the reversible interaction with the support or lawn, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

For example, suitable linkers can include: the functional group participating in or formed by the bond to the framework, the functional group or groups participating in or formed by the reversible interaction with the support or lawn, and a linker backbone moiety. The linker backbone moiety can include about 4 to about 48 carbon or heteroatoms, about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms, or the like. The linker backbone can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, mixtures thereof, or like moiety.

In an embodiment, the linker includes a lipophilic moiety, the functional group participating in or formed by the bond to the framework, and, optionally, one or more moieties for forming a reversible covalent bond, a hydrogen bond, or an ionic interaction. In such an embodiment, the lipophilic moiety can have about 4 to about 48 carbons, about 8 to about 14 carbons, about 12 to about 24 carbons, about 16 to about 18 carbons, or the like. In such an embodiment, the linker can include about 1 to about 8 reversible bond/interaction moieties or about 2 to about 4 reversible bond/interaction moieties. Suitable linkers have structures such as (CH₂)ₙCOOH, with n=12-24, n=17-24, or n=16-18.

Embodiments of Building Blocks

In an embodiment, building blocks can be represented by Formula 1: 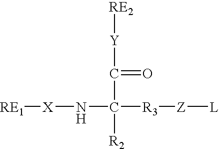
in which: RE₁ is recognition element 1, RE₂ is recognition element 2, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z is CH2, O, NH, S, CO, NR, NR₂, NHCONH, SCONH, CH═N, or OCH₂NH. In an embodiment, Z is O. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, n=16-18, n=12-16, n=12-14, or n=12.

In an embodiment, RE₁ is B1, B2, B3, B3a, B4, B5, B6, B7, B8, B9, A1, A2, A3, A3a, A4, A5, A6, A7, A8, or A9. In an embodiment, RE₁ is B1, B2, B3, B3a, B4, B5, B6, B7, B8, or B9. In an embodiment, RE₂ is A1, A2, A3, A3a, A4, A5, A6, A7, A8, A9, B1, B2, B3, B3a, B4, B5, B6, B7, B8, or B9. In an embodiment, RE₁ can be B2, B3a, B4, B5, B6, B7, or B8. In an embodiment, RE₂ can be A2, A3a, A4, A5, A6, A7, or A8.

Embodiments of such building blocks include:
4-{4-[(Acetylamino-ethylcarbamoyl-methyl)-amino]-phenoxy}-N-dodecyl-butyramide;
4-(4-{[(3-Cyclopentyl-propionylamino)-ethylcarbamoyl-methyl]-amino}-phenoxy)-N-dodecyl-butyramide;
4-[4-({[2-(3-Chloro-phenyl)-acetylamino]-ethylcarbamoyl-methyl}-amino)-phenoxy]-N-dodecyl-butyramide;
N-{[4-(3-Dodecylcarbamoyl-propoxy)-phenylamino]-ethylcarbamoyl-methyl}-3-phenyl-acrylamide;
N-Dodecyl-4-(4-{[ethylcarbamoyl-(3-pyridin-3-yl-propionylamino)-methyl]-amino}-phenoxy)-butyramide;
N-Dodecyl-4-(4-{[ethylcarbamoyl-(2-methylsulfanyl-acetylamino)-methyl]-amino}-phenoxy)-butyramide;
N-{[4-(3-Dodecylcarbamoyl-propoxy)-phenylamino]-ethylcarbamoyl-methyl}-3-hydroxy-butyramide;
N-{[4-(3-Dodecylcarbamoyl-propoxy)-phenylamino]-ethylcarbamoyl-methyl}-succinamide;
4-(4-{[(4-Dimethylamino-butyrylamino)-ethylcarbamoyl-methyl]-amino}-phenoxy)-N-dodecyl-butyramide;
4-{4-[(Acetylamino-isobutylcarbamoyl-methyl)-amino]-phenoxy}-N-dodecyl-butyramide;
4-(4-{[(3-Cyclopentyl-propionylamino)-isobutylcarbamoyl-methyl]-amino}-phenoxy)-N-dodecyl-butyramide;
4-[4-({[2-(3-Chloro-phenyl)-acetylamino]-isobutylcarbamoyl-methyl}-amino)-phenoxy]-N-dodecyl-butyramide;
N-{[4-(3-Dodecylcarbamoyl-propoxy)-phenylamino]-isobutylcarbamoyl-methyl}-3-phenyl-acrylamide;
N-Dodecyl-4-(4-{[isobutylcarbamoyl-(3-pyridin-3-yl-propionylamino)-methyl]-amino}-phenoxy)-butyramide;
N-Dodecyl-4-(4-{[isobutylcarbamoyl-(2-methylsulfanyl-acetylamino)-methyl]-amino}-phenoxy)-butyramide;
N-{[4-(3-Dodecylcarbamoyl-propoxy)-phenylamino]-isobutylcarbamoyl-methyl}-3-hydroxy-butyramide;
N-{[3-(3-Dodecylcarbamoyl-propoxy)-phenylamino]-isobutylcarbamoyl-methyl}-succinamide;
4-(4-{[(4-Dimethylamino-butyrylamino)-isobutylcarbamoyl-methyl]-amino}-phenoxy)-N-dodecyl-butyramide;
4-{4-[(Acetylamino-phenethylcarbamoyl-methyl)-amino]-phenoxy}-N-dodecyl-butyramide;
4-(4-{[(3-Cyclopentyl-propionylamino)-phenethylcarbamoyl-methyl]-amino}-phenoxy)-N-dodecyl-butyramide;
4-(4-{[[2-(3-Chloro-phenyl)-acetylamino]-(3-methyl-hexa-3,5-dienylcarbamoyl)-methyl]-amino}-phenoxy)-N-dodecyl-butyramide;
N-{[4-(3-Dodecylcarbamoyl-propoxy)-phenylamino]-phenethylcarbamoyl-methyl}-3-phenyl-acrylamide;
N-Dodecyl-4-(4-{[phenethylcarbamoyl-(3-pyridin-3-yl-propionylamino)-methyl]-amino}-phenoxy)-butyramide;
N-Dodecyl-4-(4-{[(2-methylsulfanyl-acetylamino)-phenethylcarbamoyl-methyl]-amino}-phenoxy)-butyramide;
N-{[4-(3-Dodecylcarbamoyl-propoxy)-phenyl amino]-phenethylcarbamoyl-methyl}-3-hydroxy-butyramide;
N-{[4-(3-Dodecylcarbamoyl-propoxy)-phenylamino]-phenethylcarbamoyl-methyl}-succinamide;
4-(4-{[(4-Dimethylamino-butyrylamino)-phenethylcarbamoyl-methyl]-amino}-phenoxy)-N-dodecyl-butyramide;
4-[4-({Acetylamino-[2-(4-methoxy-phenyl)-ethylcarbamoyl]-methyl}-amino)-phenoxy]-N-dodecyl-butyramide;
4-[4-({(3-Cyclopentyl-propionylamino)-[2-(4-methoxy-phenyl)-ethylcarbamoyl]-methyl}-amino)-phenoxy]-N-dodecyl-butyramide;
4-[4-({[2-(3-Chloro-phenyl)-acetylamino]-[2-(4-methoxy-phenyl)-ethylcarbamoyl]-methyl}-amino)-phenoxy]-N-dodecyl-butyramide;
N-{[4-(3-Dodecylcarbamoyl-propoxy)-phenylamino]-[2-(4-methoxy-phenyl)-ethylcarbamoyl]-methyl}-3-phenyl-acrylamide;
N-Dodecyl-4-(4-{[[2-(4-methoxy-phenyl)-ethylcarbamoyl]-(3-pyridin-3-yl-propionylamino)-methyl]-amino}-phenoxy)-butyramide;
N-Dodecyl-4-(4-{[[2-(4-methoxy-phenyl)-ethylcarbamoyl]-(2-methylsulfanyl-acetylamino)-methyl]-amino}-phenoxy)-butyramide;
N-{[4-(3-Dodecylcarbamoyl-propoxy)-phenylamino]-[2-(4-methoxy-phenyl)-ethylcarbamoyl]-methyl}-3-hydroxy-butyramide;
N-{[4-(3-Dodecylcarbamoyl-propoxy)-phenylamino]-[2-(4-methoxy-phenyl)-ethylcarbamoyl]-methyl}-succinamide;
4-[4-({(4-Dimethylamino-butyrylamino)-[2-(4-methoxy-phenyl)-ethylcarbamoyl]-methyl}-amino)-phenoxy]-N-dodecyl-butyramide;
4-(4-{[Acetylamino-(2-pyridin-2-yl-ethylcarbamoyl)-methyl]-amino}-phenoxy)-N-dodecyl-butyramide;
4-(4-{[(3-Cyclopentyl-propionylamino)-(2-pyridin-2-yl-ethylcarbamoyl)-methyl]-amino}-phenoxy)-N-dodecyl-butyramide;
4-(4-{[[2-(3-Chloro-phenyl)-acetylamino]-(2-pyridin-2-yl-ethylcarbamoyl)-methyl]-amino}-phenoxy)-N-dodecyl-butyramide;
N-[[4-(3-Dodecylcarbamoyl-propoxy)-phenylamino]-(2-pyridin-2-yl-ethylcarbamoyl)-methyl]-3-phenyl-acrylamide;
N-Dodecyl-4-(4-{[(2-pyridin-2-yl-ethylcarbamoyl)-(3-pyridin-3-yl-propionylamino)-methyl]-amino}-phenoxy)-butyramide
N-Dodecyl-4-(4-{[(2-methylsulfanyl-acetylamino)-(2-pyridin-2-yl-ethylcarbamoyl)-methyl]-amino}-phenoxy)-butyramide;
N-[[4-(3-Dodecylcarbamoyl-propoxy)-phenylamino]-(2-pyridin-2-yl-ethylcarbamoyl)-methyl]-3-hydroxy-butyramide;
N-[[4-(3-Dodecylcarbamoyl-propoxy)-phenylamino]-(2-pyridin-2-yl-ethylcarbamoyl)-methyl]-succinamide;
4-(4-{[(4-Dimethylamino-butyrylamino)-(2-pyridin-2-yl-ethylcarbamoyl)-methyl]-amino}-phenoxy)-N-dodecyl-butyramide;
4-(4-{[Acetylamino-(2-methoxy-ethylcarbamoyl)-methyl]-amino}-phenoxy)-N-dodecyl-butyramide;
4-(4-{[(3-Cyclopentyl-propionylamino)-(2-methoxy-ethylcarbamoyl)-methyl]-amino}-phenoxy)-N-dodecyl-butyramide;
4-(4-{[[2-(3-Chloro-phenyl)-acetylamino]-(2-methoxy-ethylcarbamoyl)-methyl]-amino}-phenoxy)-N-dodecyl-butyramide;
N-[[4-(3-Dodecylcarbamoyl-propoxy)-phenylamino]-(2-methoxy-ethylcarbamoyl)-methyl]-3-phenyl-acrylamide;
N-Dodecyl-4-(4-{[(2-methoxy-ethylcarbamoyl)-(3-pyridin-3-yl-propionylamino)-methyl]-amino}-phenoxy)-butyramide;
N-Dodecyl-4-(4-{[(2-methoxy-ethylcarbamoyl)-(2-methylsulfanyl-acetylamino)-methyl]-amino}-phenoxy)-butyramide;
N-[[4-(3-Dodecylcarbamoyl-propoxy)-phenylamino]-(2-methoxy-ethylcarbamoyl)-methyl]-3-hydroxy-butyramide;
N-[[3-(3-Dodecylcarbamoyl-propoxy)-phenylamino]-(2-methoxy-ethylcarbamoyl)-methyl]-succinamide;
4-(4-{[(4-Dimethylamino-butyrylamino)-(2-methoxy-ethylcarbamoyl)-methyl]-amino}-phenoxy)-N-dodecyl-butyramide;
4-(4-{[Acetylamino-(2-hydroxy-ethylcarbamoyl)-methyl]-amino}-phenoxy)-N-dodecyl-butyramide;
4-(4-{[(3-Cyclopentyl-propionylamino)-(2-hydroxy-ethylcarbamoyl)-methyl]-amino}-phenoxy)-N-dodecyl-butyramide;
4-(4-{[[2-(3-Chloro-phenyl)-acetylamino]-(2-hydroxy-ethylcarbamoyl)-methyl]-amino}-phenoxy)-N-dodecyl-butyramide;
N-[[4-(3-Dodecylcarbamoyl-propoxy)-phenylamino]-(2-hydroxy-ethylcarbamoyl)-methyl]-3-phenyl-acrylamide;
N-Dodecyl-4-(4-{[(2-hydroxy-ethylcarbamoyl)-(3-pyridin-3-yl-propionylamino)-methyl]-amino}-phenoxy)-butyramide;
N-Dodecyl-4-(4-{[(2-hydroxy-ethylcarbamoyl)-(2-methylsulfanyl-acetylamino)-methyl]-amino}-phenoxy)-butyramide;
N-[[4-(3-Dodecylcarbamoyl-propoxy)-phenylamino]-(2-hydroxy-ethylcarbamoyl)-methyl]-3-hydroxy-butyramide;
N-[[3-(3-Dodecylcarbamoyl-propoxy)-phenylamino]-(2-hydroxy-ethylcarbamoyl)-methyl]-succinamide;
4-(4-{[(4-Dimethylamino-butyrylamino)-(2-hydroxy-ethylcarbamoyl)-methyl]-amino}-phenoxy)-N-dodecyl-butyramide;
4-(4-{[Acetylamino-(2-acetylamino-ethylcarbamoyl)-methyl]-amino}-phenoxy)-N-dodecyl-butyramide;
4-(4-{[(2-Acetylamino-ethylcarbamoyl)-(3-cyclopentyl-propionylamino)-methyl]-amino}-phenoxy)-N-dodecyl-butyramide;
4-[4-({(2-Acetylamino-ethylcarbamoyl)-[2-(3-chloro-phenyl)-acetylamino]-methyl}-amino)-phenoxy]-N-dodecyl-butyramide;
N-{(2-Acetylamino-ethylcarbamoyl)-[4-(3-dodecylcarbamoyl-propoxy)-phenylamino]-methyl}-3-phenyl-acrylamide;
4-(4-{[(2-Acetylamino-ethylcarbamoyl)-(3-pyridin-3-yl-propionylamino)-methyl]-amino}-phenoxy)-N-dodecyl-butyramide;
4-(4-{[(2-Acetylamino-ethylcarbamoyl)-(2-methylsulfanyl-acetylamino)-methyl]-amino}-phenoxy)-N-dodecyl-butyramide;
N-{(2-Acetylamino-ethylcarbamoyl)-[4-(3-dodecylcarbamoyl-propoxy)-phenylamino]-methyl}-3-hydroxy-butyramide;
N-{(2-Acetylamino-ethylcarbamoyl)-[3-(3-dodecylcarbamoyl-propoxy)-phenylamino]-methyl}-succinamide;
4-(4-{[(2-Acetylamino-ethylcarbamoyl)-(4-dimethylamino-butyrylamino)-methyl]-amino}-phenoxy)-N-dodecyl-butyramide;
4-(4-{[Acetylamino-(2-pyrrolidin-1-yl-ethylcarbamoyl)-methyl]-amino}-phenoxy)-N-dodecyl-butyramide;
4-(4-{[(3-Cyclopentyl-propionylamino)-(2-pyrrolidin-1-yl-ethylcarbamoyl)-methyl]-amino}-phenoxy)-N-dodecyl-butyramide;
4-(4-{[[2-(3-Chloro-phenyl)-acetylamino]-(2-pyrrolidin-1-yl-ethylcarbamoyl)-methyl]-amino}-phenoxy)-N-dodecyl-butyramide;
N-[[4-(3-Dodecylcarbamoyl-propoxy)-phenylamino]-(2-pyrrolidin-1-yl-ethylcarbamoyl)-methyl]-3-phenyl-acrylamide;
N-Dodecyl-4-(4-{[(3-pyridin-3-yl-propionylamino)-(2-pyrrolidin-1-yl-ethylcarbamoyl)-methyl]-amino}-phenoxy)-butyramide;
N-Dodecyl-4-(4-{[(2-methylsulfanyl-acetylamino)-(2-pyrrolidin-1-yl-ethylcarbamoyl)-methyl]-amino}-phenoxy)-butyramide;
N-[[4-(3-Dodecylcarbamoyl-propoxy)-phenylamino]-(2-pyrrolidin-1-yl-ethylcarbamoyl)-methyl]-3-hydroxy-butyramide;
N-[[3-(3-Dodecylcarbamoyl-propoxy)-phenylamino]-(2-pyrrolidin-1-yl-ethylcarbamoyl)-methyl]-succinamide;
4-(4-{[(4-Dimethylamino-butyrylamino)-(2-pyrrolidin-1-yl-ethylcarbamoyl)-methyl]-amino}-phenoxy)-N-dodecyl-butyramide;
salts thereof, esters thereof, protected or blocked derivatives thereof, immobilized derivatives thereof, derivatives thereof, or mixtures thereof. The nomenclature in this paragraph is according to the program CS CHEMDRAW ULTRA®.

Building blocks including an A and/or a B recognition element, a linker, and an amino acid framework can be made by methods illustrated in general Scheme 1. 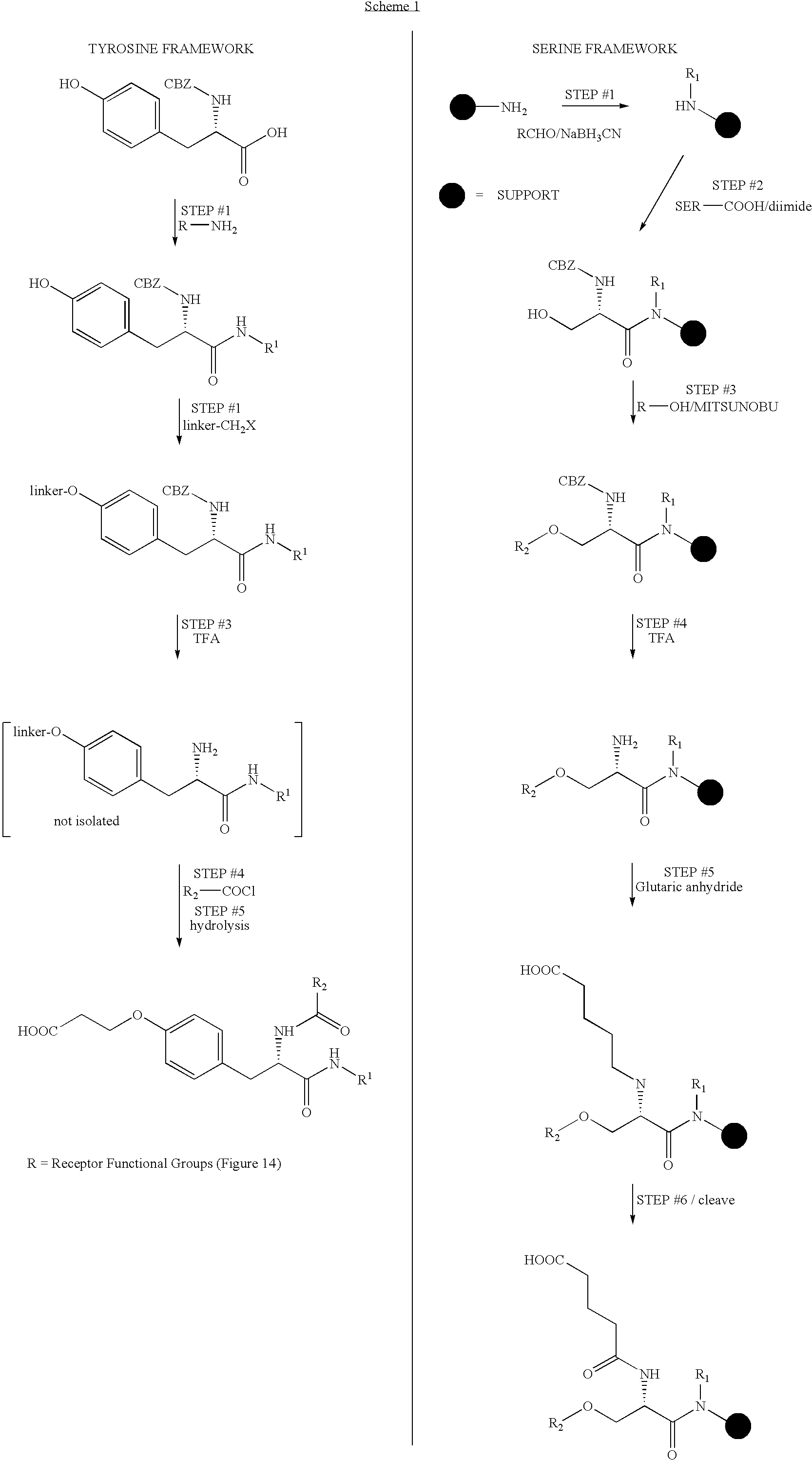
Embodiments of Building Blocks Reversibly Immobilized on Lawn or Support

The present invention includes building blocks reversibly immobilized on a lawn or a support through any of a variety of interactions or combination of interactions described above. In an embodiment, the functionalized lawn includes a first covalent bonding moiety and the building block includes a second covalent bonding moiety. The first and second covalent bonding moieties can form or can be coupled by a readily reversible covalent bond. In an embodiment, the first covalent bonding moiety includes an amine nitrogen and the second covalent bonding moiety includes a carbonyl carbon. In an embodiment, the first covalent bonding moiety includes a carbonyl carbon and the second covalent bonding moiety includes an amine nitrogen.

In an embodiment, the first covalent bonding moiety includes an amine nitrogen and the second covalent bonding moiety includes a carbonyl carbon; the first covalent bonding moiety includes a carbonyl carbon and the second covalent bonding moiety includes an amine nitrogen; or a mixture or a combination thereof.

In an embodiment, the functionalized lawn includes a first charged moiety and the building block includes a second charged moiety. In such an embodiment, the first and second charged moieties advantageously have opposite charges. In an embodiment, the first charged moiety includes a carboxylate and the second charged moiety includes an ammonium. In an embodiment, the first charged moiety includes an ammonium and the second charged moiety includes a carboxylate.

In an embodiment, the first charged moiety includes a carboxylate and the second charged moiety includes an ammonium; the first charged moiety includes an ammonium and the second charged moiety includes a carboxylate; or a mixture or a combination thereof.

In an embodiment, the functionalized lawn includes a first lipophilic moiety and the building block includes a second lipophilic moiety. In an embodiment, the first and second lipophilic moieties includes independently one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like.

In an embodiment, the functionalized lawn includes a first lipophilic moiety and a first covalent bonding moiety; and the building block includes a second lipophilic moiety and a second covalent bonding moiety. In an embodiment, the functionalized lawn includes a first lipophilic moiety and a first charged moiety; and the building block includes a second lipophilic moiety and a second charged moiety. In an embodiment, the functionalized lawn includes a first lipophilic moiety and a first covalent bonding moiety and the building block includes a second lipophilic moiety and a second covalent bonding moiety; the functionalized lawn includes a first lipophilic moiety and a first charged moiety; and the building block includes a second lipophilic moiety and a second charged moiety; or a combination or a combination thereof.

In an embodiment, the present invention includes a heterogeneous building block array. Such a building block array can include a support, a functionalized lawn, and a plurality of building blocks. The functionalized lawn can be coupled to the support. The array can include a plurality of regions on the support, and the regions can include a plurality of building blocks. In this embodiment, the plurality of building blocks can be reversibly immobilized on the lawn.

In an embodiment, the present invention includes a composition. Such a composition can include a support, a functionalized lawn, and a plurality of building blocks. The functionalized lawn can be coupled to the surface, and a region on the surface can include a plurality of building blocks. In this embodiment, the building blocks can be reversibly immobilized on the lawn.

Embodiments of Sets or Kits of Reagents

The present invention includes compositions, articles of manufacture, kits, and reagents that can make, form, or include artificial receptors, such as candidate artificial receptors. Such an artificial receptor can include or be part of a dynamic building block array.

In an embodiment, the present invention includes an article of manufacture. Such an article of manufacture can include a support, a functionalized lawn reagent, and a plurality of building blocks. The functionalized lawn can be configured to be coupled to the support. The plurality of building blocks can be configured to be reversibly coupled to the lawn. For example, the functionalized lawn reagent can include a first covalent bonding moiety and the building block comprises a second covalent bonding moiety. For example, the functionalized lawn reagent can include a first charged moiety and the building block comprises a second charged moiety, the first and second charged moieties having opposite charges. For example, the functionalized lawn reagent can include a first lipophilic moiety and the building block comprises a second lipophilic moiety. The article of manufacture can include a functionalized glass support.

Building Blocks and/or Lawns on Supports

Forming a spot on a support can be accomplished by methods and apparatus such as pin spotters (sometimes referred to as printers), which can, for example, spot 10,000 to more than 100,000 spots on a microscope slide. Other spotters include piezoelectric spotters (similar to ink jets) and electromagnetic spotters that can also spot, for example, 10,000 to more than 100,000 spots on a microscope slide. An array of spots can also be printed on the bottom of a well of a microtiter plate. Arrays can also be built using photolithography and other known processes that can produce spots containing building blocks on a substrate. Conventional mixing valves or manifolds can be employed to mix the activated building blocks before spotting. These valves or manifolds can be under control of conventional microprocessor based controllers for selecting building blocks and amounts of reagents. Alternatively, the activated building blocks can be provided as mixtures made, for example, in large numbers in microwell plates by a robotic system.

Such spotting yields a microarray of spots of heterogeneous combinations of building blocks, each of which can be a candidate artificial receptor. Each spot in a microarray includes a statistically significant number of each building block. For example, although not limiting to the present invention, it is believed that each micro spot of a size sufficiently small that 100,000 fit on a microscope slide can include approximately 320 million clusters of 4 building blocks.

In an embodiment, the present method includes making a receptor surface. Making a receptor surface can include forming a region on a solid support, the region including a plurality of building blocks, and coupling the plurality of building blocks to the solid support in the region. The method can include mixing a plurality of activated building blocks and employing the mixture in forming the region or regions. Alternatively, the method can include applying individual activated building blocks in a region on the support. Forming a region on a support can be accomplished, for example, by soaking a portion of the support with the building block solution.

A region including a plurality of building blocks can be independent and distinct from other regions including a plurality of building blocks. In an embodiment, one or more regions including a plurality of building blocks can overlap to produce a region including the combined pluralities of building blocks. In an embodiment, two or more regions including a single building block can overlap to form one or more regions each including a plurality of building blocks. The overlapping regions can be envisioned, for example, as portions of overlap in a Ven diagram, or as portions of overlap in a pattern like a plaid or tweed.

In an embodiment, a tube or well coated with a support matrix can be filled with activated building block (e.g., a solution containing activated building block), which couples to the support matrix. For example, the support can be a glass tube or well coated with a plurality of building blocks. The surface of the glass tube or well can be coated with a coating to which the plurality of building blocks become covalently bound. The resulting coating including building blocks can be referred to as including heterogeneous building blocks.

In an embodiment, the method produces a surface or coating with a density of building blocks sufficient to provide interactions of more than one building block with a ligand. That is, the building blocks can be in proximity to one another. Proximity of different building blocks can be detected by determining different (e.g., greater) binding of a test ligand to a surface including a plurality of building blocks compared to a surface or surfaces including only one of the building blocks.

In an embodiment, the present method can be employed to produce a solid support having on its surface a plurality of regions or spots, each region or spot including a plurality of building blocks. For example, the method can include spotting a glass slide with a plurality of spots, each spot including a plurality of building blocks. In an embodiment, the spots include 2, 3, 4, 5, or 6 building blocks. Such a spot can be referred to as including heterogeneous building blocks.

Each spot can include a density of building blocks sufficient to provide interactions of more than one building block with a ligand. Such interactions can be determined as described above for regions. The method can include spotting the building blocks so that each spot is separated from the others. A plurality of spots of building blocks is referred to herein as an array of spots. In an embodiment, an array of spots can include more than 1 million spots.

In an embodiment, the method includes forming an array including one or more spots that function as controls for validating or evaluating binding to artificial receptors of the present invention. In an embodiment, the method includes forming one or more regions, tubes, or wells that function as controls for validating or evaluating binding to artificial receptors of the present invention. Such a control spot, region, tube, or well can include no building block, only a single building block, only functionalized lawn, or combinations thereof.

The method can employ any of the variety of known supports employed in combinatorial or synthetic chemistry (e.g., a microscope slide, a bead, a resin, a gel, or the like). Suitable supports include functionalized glass, such as a functionalized slide or tube, glass microscope slide, glass plate, glass coverslip, glass beads, microporous glass beads, microporous polymer beads (e.g. those sold under the tradename Stratospheres™), silica gel supports, and the like. Suitable supports also include plates with wells, such as 96 or 384 well microplates. Suitable supports with hydrophobic surfaces include micelles and reverse micelles.

The support can include a support matrix of a compound or mixture of compounds having functional groups suitable for reversibly coupling to a building block or coupling to a lawn reagent. The support matrix can be, for example, a coating on a microscope slide or functionalizing groups on a bead, gel, or resin. Known support matrices are commercially available and/or include linkers with functional groups that are coupled beads, gels, or resins. The support matrix functional groups can be pendant from the support in groups of one (e.g., as a lawn of amines, a lawn of another functional group, or a lawn of a mixture of functional groups) or in groups of, for example, 2, 3, 4, 5, 6, or 7.

A commercially available glass support can be prepared for coupling building blocks by adding a support matrix to the surface of the support. The support matrix provides functional groups for coupling to the building block. Suitable support matrices include silanating agents. Starting with a commercially available slide, an amino functionalized slide from Corning, building blocks including an activated ester can be spotted on and covalently bound to the slide in a micro array by this same reaction.

The method can couple the lawn reagent to a support using known methods for activating compounds of the types employed as lawn reagent and for coupling them to supports. Covalent coupling can produce lawns on supports that are sufficiently durable to be used repeatedly over a period of months. The method can employ lawn reagent including activated esters and couple them to supports including amine functional groups. The method can include activating a carboxyl group on a lawn reagent by derivatizing to form the activated ester. By way of further example, the method can couple lawn reagent including amine functional groups to supports including carboxyl groups. Pairs of functional groups that can be employed on lawn reagent and support according to the present invention include nucleophile/electrophile pairs, such as amine and carboxyl (or activated carboxyl), thiol and maleimide, alcohol and carboxyl (or activated carboxyl), mixtures thereof, and the like.

The support can include any functional group suitable for forming a covalent bond with a lawn reagent. The support or the lawn reagent can include a functional group such as alcohol, phenol, thiol, amine, carbonyl, or like group. The support or the lawn reagent can include a carboxyl, alcohol, phenol, thiol, amine, carbonyl, maleimide, or like group that can react with or be activated to react with the support or the building block. The support can include one or more of these groups. The lawn reagent can include a plurality of these groups.

The support or the lawn reagent can include a good leaving group bonded to, for example, an alkyl or aryl group. The leaving group being “good” enough to be displaced by the alcohol, phenol, thiol, amine, carbonyl, or like group on the support or the lawn reagent. Such a support or lawn reagent can include a moiety represented by the formula: R—X, in which X is a leaving group such as halogen (e.g., —Cl, —Br, or —I), tosylate, mesylate, or triflate, and R is alkyl, substituted alkyl, cycloalkyl, heterocyclic, substituted heterocyclic, aryl alkyl, aryl, heteroaryl, or heteroaryl alkyl. The support can include one or more of these groups. The lawn reagent can include a plurality of these groups.

An amine modified glass surface can be functionalized with lawn reagent, for example, by reaction with activated carboxyl derivatives to form an amide link to the functionalized support. For example, a lawn reagent carboxyl group can be activated by reacting the lawn reagent with carbodiimide in the presence of sulfo N-hydroxysuccinimide in aqueous dimethylformamide. The activated lawn reagent can be reacted directly with an amine on a glass support (hereinafter amino glass). Derivatization of only a portion of the amine groups on the support can be effective for producing candidate artificial receptors. Although not limiting to the present invention, it is believed that the amine load on the glass is in excess of that required for candidate artificial receptor preparation.

A lawn or other coating of functional groups can be derivatized with a maximum density of lawn reagent by exposing the support to several equivalents of lawn reagent. For example, less than 1 (e.g., 0.1) or more (e.g., 10) equivalents can sufficient for an adequate density of lawn reagent on the support.

In an embodiment, the candidate artificial receptor can include lawn, building blocks, and unmodified amines (or other functional groups) on the support. In an embodiment, the candidate artificial receptor can include lawn, building blocks, and modified amines on the support. For example, the amines on a support can be modified by the simplest amide modification of the amines to form the acetamide (e.g., by reacting with acetic anhydride or acetyl chloride). Alternatively, the amines of the support can be modified by reaction with succinic anhydride, benzoyl chloride, or the like. In an embodiment, the support can be modified with or the lawn can include a signal element that produces a detectable signal when a test ligand is bound to the receptor. For example, the signal element can be a fluorescent molecule that is quenched by binding to the artificial receptor. For example, the signal element can be a molecule that fluoresces only when binding occurs.

In an embodiment, the present methods, compositions, artificial receptors, and articles of manufacture can include or make artificial receptors including reversibly immobilized building blocks and building blocks that are more stringently immobilized. For example, more stringently immobilized building blocks can be coupled to the lawn or the support as described above for coupling lawn to support. The method can couple the more stringently immobilized building blocks to a support using known methods for activating compounds of the types employed as building blocks and for coupling them to supports. Covalent coupling can produce building blocks on supports that are sufficiently durable to be used repeatedly over a period of months.

The present methods and compositions can employ building blocks including activated esters and couple them to supports including amine functional groups. The method can include activating a carboxyl group on a building block by derivatizing to form the activated ester. By way of further example, the method can couple building block including amine functional groups to supports including carboxyl groups. Pairs of functional groups that can be employed on building block and support for more stringent coupling, according to the present invention, include nucleophile/electrophile pairs, such as amine and carboxyl (or activated carboxyl), thiol and maleimide, alcohol and carboxyl (or activated carboxyl), mixtures thereof, and the like.

The support can include any functional group suitable for forming a covalent bond with a building block. The support or the building block can include a functional group such as alcohol, phenol, thiol, amine, carbonyl, or like group. The support or the building block can include a carboxyl, alcohol, phenol, thiol, amine, carbonyl, maleimide, or like group that can react with or be activated to react with the support or the building block. The support can include one or more of these groups. The building block can include a plurality of these groups.

The support or the building block can include a good leaving group bonded to, for example, an alkyl or aryl group. The leaving group being “good” enough to be displaced by the alcohol, phenol, thiol, amine, carbonyl, or like group on the support or the building block. Such a support or building block can include a moiety represented by the formula: R—X, in which X is a leaving group such as halogen (e.g., —Cl, —Br, or —I), tosylate, mesylate, or triflate, and R is alkyl, substituted alkyl, cycloalkyl, heterocyclic, substituted heterocyclic, aryl alkyl, aryl, heteroaryl, or heteroaryl alkyl. The support can include one or more of these groups. The building block can include a plurality of these groups.

An amine modified glass surface can be reacted with building block by reaction with activated carboxyl derivatives to form an amide link to the functionalized support. For example, a building block carboxyl group can be activated by reacting the building block with carbodiimide in the presence of sulfo N-hydroxysuccinimide in aqueous dimethylformamide. The activated building block can be reacted directly with an amine on a glass support (hereinafter amino glass). Derivatization of only a portion of the amine groups on the support can be effective for producing candidate artificial receptors. Although not limiting to the present invention, it is believed that the amine load on the glass is in excess of that required for candidate artificial receptor preparation.

A lawn or other coating of functional groups can be derivatized with a maximum density of building blocks by exposing the lawn to several equivalents of building blocks. For example, less than 1 (e.g., 0.1) or more equivalents (e.g., up to 10) is sufficient for an adequate density of building blocks on the support to observe building-block-dependent binding of a ligand.

In an embodiment, one or more slides or supports can include heterogeneous spots or regions of building blocks made from combinations of a subset of the total building blocks and/or smaller groups of the building blocks in each spot or region. That is, each spot or region includes only, for example, 2 or 3 building blocks, rather than 4 or 5. For example, one or more slides can include the number of spots formed by combinations of a full set of building blocks (e.g. 81 of a set of 81) in groups of 2 or 3. For example, the one or more slides can include the number of spots formed by combinations of a subset of the building blocks (e.g., 25 of the set of 81) in groups of 4 or 5. For example, the one or more slides can include the number of spots formed by combinations of a subset of the building blocks (e.g., 25 of the set of 81) in groups of 2 or 3. Should a candidate artificial receptor of interest be identified from the subset and/or smaller groups, then additional subsets and groups can be made or selected incorporating the building blocks in the candidates of interest or structurally similar building blocks. These additional subsets or groups can be exchanged into the original artificial receptors or building block compositions. The content of the additional subsets or groups of building blocks can be selected, for example, to provide structural diversity or to provide various structures similar to the building blocks in the artificial receptor.

The method can apply or spot building blocks onto a support in combinations of 2 or 3 building blocks. Effective artificial receptors can be developed employing as few as several dozen or several hundred artificial receptors, that can include 2 and/or, preferably, 3 building blocks. Such artificial receptors can employ, for example, a tube, well, or slide as a support.

In an embodiment, a candidate artificial receptor can be identified from binding of a test ligand to one group of building blocks. Then, the entire set of building blocks can be exchanged with the candidate artificial receptor.

In an embodiment, the present invention includes a scaffold molecule having coupled to it a plurality of building blocks. For example, the scaffold can be a polyamine, for example, a cyclic molecule with a plurality of primary amine groups around the ring. Such a scaffold can include a plurality of building blocks coupled to the amines. Such a scaffold can be referred to as including heterogeneous building blocks. The scaffold can provide a density of building blocks sufficient to provide interactions of more than one building block with a ligand. A scaffold can be the support for an artificial receptor including a combination of 3, 4, or more building blocks occupying distinct positions relative to one another on the scaffold. For example, building block 1 can be adjacent to any of building blocks 2, 3, or 4.

The present invention includes sets of building blocks as reagents. Reagent sets of building blocks can include individual or mixtures of building blocks. The reagent sets can be used to make immobilized building blocks and groups of building blocks, and can be sold for this purpose. In an embodiment, the set includes building blocks with recognition elements representing hydrophobic alkyl, hydrophobic aryl, hydrogen bond acceptor, basic, hydrogen bond donor, and small size as structural characteristics. The set can be part of a kit including containers of one or mixtures of building blocks, the containers can be in a package, and the kit can include written material describing the building blocks and providing instructions for their use.

Using the Artificial Receptors

The present invention includes a method of using artificial receptors. The present invention includes a method of screening candidate artificial receptors to find lead artificial receptors that bind a particular test ligand. The method can then improve upon or test additional candidate or lead artificial receptors by allowing movement of the building blocks that make up the artificial receptors. Movement of building blocks can include mobilizing the building block to move along or on the support and/or to leave the support and enter a fluid (e.g., liquid) phase separate from the support or lawn.

In an embodiment, building blocks can be mobilized to move along or on the support (translate or shuffle). Such translation can be employed, for example, to allow building blocks already bound to a test ligand to rearrange into a lower energy or tighter binding configuration still bound to the test ligand. Such translation can be employed, for example, to allow the ligand access to building blocks that are on the support but not bound to the ligand. These building blocks can translate into proximity with and bind to a test ligand.

Building blocks can be induced to move along or on the support or to be reversibly immobilized on the support through any of a variety of mechanisms. For example, inducing mobility of building blocks can include altering the conditions of the support or lawn. That is, altering the conditions can reverse the immobilization of the building blocks, thus mobilizing them. Reversibly immobilizing the building blocks after they have moved can include, for example, returning to the previous conditions. Suitable alterations of conditions include changing pH, changing temperature, changing polarity or hydrophobicity, changing ionic strength, changing nucleophilicity or electrophilicity (e.g. of solvent or solute), and the like.

A variety of methods can be used to change the conditions of the surface or the building block. For example, fluid can be applied to the surface or lawn in an amount or of a composition that can wet and/or change the conditions of the surface or lawn without providing bulk fluid into which building blocks can exchange. In an embodiment, the amount of fluid is sufficient to hydrate the surface or lawn without leaving any bulk solvent. In an embodiment, translation or shuffling can be achieved without exchanging by change in temperature, or the like.

A building block reversibly immobilized by hydrophobic interactions can be mobilized by increasing the temperature, by exposing the surface, lawn, or building block to a more hydrophobic solvent (e.g., an organic solvent or a surfactant), or by reducing ionic strength around the building block. In an embodiment, the organic solvent includes acetonitrile, acetic acid, an alcohol, tetrahydrofuran (THF), dimethylformamide (DMF), hydrocarbons such as hexane or octane, acetone, chloroform, methylene chloride, or the like, or mixture thereof. In an embodiment, the surfactant includes a nonionic surfactant, such as a nonylphenol ethoxylate, or the like. A building block that is mobile on a support can be reversibly immobilized by hydrophobic interactions, for example, by decreasing the temperature, exposing the surface, lawn, or building block to a more hydrophilic solvent (e.g., an aqueous solvent) or increased ionic strength.

A building block reversibly immobilized by hydrogen bonding can be mobilized by increasing the ionic strength, concentration of hydrophilic solvent, or concentration of a competing hydrogen bonder in the environs of the building block. A building block that is mobile on a support can be reversibly immobilized through an ionic interaction by decreasing ionic strength of the hydrophilic solvent, or the like.

A building block reversibly immobilized by an ionic interaction can be mobilized by increasing the ionic strength in the environs of the building block. Increasing ionic strength can disrupt ionic interactions. A building block that is mobile on a support can be reversibly immobilized through an ionic interaction by decreasing ionic strength.

A building block reversibly immobilized by an imine, acetal, or ketal bond can be mobilized by decreasing the pH or increasing concentration of a nucleophilic catalyst in the environs of the building block. In an embodiment, the pH is about 1 to about 4. Imines, acetals, and ketals undergo acid catalyzed hydrolysis. A building block that is mobile on a support can be reversibly immobilized by a reversible covalent interaction, such as by forming an imine, acetal, or ketal bond, by increasing the pH.

In an embodiment, building blocks can be mobilized to leave the support and enter a fluid (e.g., liquid) phase separate from the support or lawn (exchange). For example, building blocks can be exchanged onto and/or off of the support. Exchange can be employed, for example, to allow building blocks on a support but not bound to a test ligand to be removed from the support. Exchange can be employed, for example, to add additional building blocks to the support. The added building blocks can have structures selected based on knowledge of the structures of the building blocks in artificial receptors that bind the test ligand. The added building blocks can have structures selected to provide additional structural diversity. The added building blocks can include all of the building blocks.

Building blocks can be induced to exchange on to and/or off of the support through any of a variety of mechanisms. For example, inducing exchange of building blocks can include contacting the building block with fluid. In an embodiment, contacting employs sufficient volume of the fluid to dilute the building block from the support. In an embodiment, contacting employs an amount and type of fluid that extracts the building block from the support. The contacting fluid can include reagents or have a characteristic that can reverse the immobilization of the building blocks, thus allowing them to exchange. In an embodiment, contacting employs a fluid containing a building block to be added to the support. The contacting fluid can include a reagent or have a characteristic that promotes reversible immobilization of the building blocks on the support.

For example, the fluid can have a pH, temperature, polarity or hydrophobicity, ionic strength, nucleophilicity or electrophilicity, and the like that promotes release of the building blocks from the support. Alternatively, the fluid can have a pH, temperature, polarity or hydrophobicity, ionic strength, nucleophilicity or electrophilicity, and the like that promotes reversible immobilization of the building blocks on the support.

A building block reversibly immobilized by hydrophobic interactions can be released from the support by, for example, raising the temperature, e.g., of the support and/or artificial receptor. For example, the hydrophobic interactions (e.g., the hydrophobic group on the support or lawn and on the building block) can be selected to provide immobilized building block at about room temperature or below and release can be accomplished at a temperature above room temperature. For example, the hydrophobic interactions can be selected to provide immobilized building block at about refrigerator temperature (e.g., 4° C.) or below and release can be accomplished at a temperature of, for example, room temperature or above. By way of further example, a building block can be reversibly immobilized by hydrophobic interactions, for example, by contacting the surface or artificial receptor with a fluid containing the building block and that is at or below room temperature.

A building block reversibly immobilized by hydrophobic interactions can be released from the support by, for example, contacting the artificial receptor with a sufficiently hydrophobic fluid (e.g., an organic solvent or a surfactant). In an embodiment, the organic solvent includes acetonitrile, acetic acid, an alcohol, tetrahydrofuran (THF), dimethylformamide (DMF), hydrocarbons such as hexane or octane, acetone, chloroform, methylene chloride, or the like, or mixture thereof. In an embodiment, the surfactant includes a nonionic surfactant, such as a nonylphenol ethoxylate, or the like. Such reversible immobilization can also be effected by contacting the surface or artificial receptor with a hydrophilic solvent and allowing the somewhat lipophilic building block to partition on to the hydrophobic surface or lawn.

A building block reversibly immobilized by an imine, acetal, or ketal bond can be released from the support by, for example, contacting the artificial receptor with fluid having an acid pH or including a nucleophilic catalyst. In an embodiment, the pH is about 1 to about 4. A building block can be reversibly immobilized by a reversible covalent interaction, such as by forming an imine, acetal, or ketal bond, by contacting the surface or artificial receptor with fluid having a neutral or basic pH.

A building block reversibly immobilized by an ionic interaction can be released by, for example, contacting the artificial receptor with fluid having sufficiently high ionic strength to disrupt the ionic interaction. A building block can be reversibly immobilized through an ionic interaction by contacting the surface or artificial receptor with fluid having ionic strength that promotes ionic interaction between the building block and the support and/or lawn.

The methods of using the present artificial receptors including reversibly immobilized building blocks can also include using artificial receptors with more permanently linked building blocks. The more permanent receptor can be employed, for example, to provide structures for lead or candidate artificial receptors including reversibly immobilized building blocks. Suitable more permanently linked artificial receptors are described in copending U.S. patent application Ser. No. 10/244,727, filed Sep. 16, 2002, and Application No. PCT/US03/05328, filed Feb. 19, 2003, each entitled “ARTIFICIAL RECEPTORS, BUILDING BLOCKS, AND METHODS”, the disclosures of which are incorporated herein by reference.

Embodiments Employing the Present Receptors

In an embodiment, the present invention includes a method of using an artificial receptor that includes translating or shuffling one or more building blocks in one or more regions on the support. Such a method can include contacting a reversibly immobilized heterogeneous molecular array with a test ligand and shuffling building blocks in one or more regions. This embodiment of the method can also include detecting binding of a test ligand to one or more regions and/or selecting one or more of the binding regions as the artificial receptor. The artificial receptor can be a lead artificial receptor. In this method, the building blocks in the array define a first set of building blocks, and the plurality of building blocks in the one or more binding regions defines one or more selected binding combinations of building blocks.

This embodiment of the method can employ an array including a support, a functionalized lawn, and a plurality of building blocks. The functionalized lawn can be coupled to the support. The array can include a plurality of regions on the support. The regions can include a plurality of building blocks. The plurality of building blocks can be reversibly immobilized on the lawn.

In an embodiment, the functionalized lawn includes a first covalent bonding moiety and the building block includes a second covalent bonding moiety. The first and second covalent bonding moieties form a readily reversible covalent bond. In this embodiment, shuffling includes contacting one or more regions to be shuffled with a composition including reagent promoting cleavage of the readily reversible covalent bond. In an embodiment, the reagent promoting cleavage has pH of about 1 to about 4.

In an embodiment, the functionalized lawn includes a first charged moiety and the building block includes a second charged moiety, the first and second charged moieties having opposite charges. In this embodiment, shuffling includes contacting one or more regions to be shuffled with a composition including reagent promoting separation of the first and second charged moieties. In an embodiment, the reagent includes salt concentration of about 0.1 to about 1 M.

In an embodiment, the functionalized lawn includes a first lipophilic moiety and the building block includes a second lipophilic moiety. In this embodiment, shuffling includes contacting one or more regions to be shuffled with a composition including lipophilic reagent. In an embodiment, the lipophilic reagent includes organic solvent, surfactant, or mixture thereof. Suitable organic solvents and surfactants include those described hereinabove.

In an embodiment, the present invention includes a method of using an artificial receptor that includes exchanging one or more building blocks onto or off of one or more regions on the support. Such a method can include contacting a reversibly immobilized heterogeneous molecular array with a test ligand and exchanging one or more building blocks onto or off of the support. This embodiment of the method can also include detecting binding of a test ligand to one or more regions and/or selecting one or more of the binding regions as the artificial receptor. The artificial receptor can be a lead artificial receptor. In this method, the building blocks in the array define a first set of building blocks, and the plurality of building blocks in the one or more binding regions defines one or more selected binding combination of building blocks.

This embodiment of the method can employ an array including a support, a functionalized lawn, and a plurality of building blocks. The functionalized lawn can be coupled to the support. The array can include a plurality of regions on the support. The regions can include a plurality of building blocks. The plurality of building blocks can be reversibly immobilized on the lawn.

In an embodiment, exchanging includes contacting one or more regions with added building block and reversibly immobilizing the added building block in the region. In an embodiment, exchanging includes contacting one or more regions with reagent promoting release of reversibly immobilized building block and removing released building block. In an embodiment, exchanging includes contacting one or more regions with reagent promoting release of reversibly immobilized building block and removing released building block; and contacting one or more regions with added building block and reversibly immobilizing the added building block in the region.

In an embodiment, the functionalized lawn includes a first covalent bonding moiety and the building block includes a second covalent bonding moiety. The first and second covalent bonding moieties form a readily reversible covalent bond. In this embodiment, exchanging can include contacting one or more regions to be exchanged with an effective volume of a fluid including reagent promoting cleavage of the readily reversible covalent bond. In this embodiment, exchanging can include contacting one or more regions to be exchanged with an effective volume of a fluid including one or more building blocks and reagent promoting formation of the readily reversible covalent bond.

In an embodiment, the functionalized lawn includes a first charged moiety and the building block includes a second charged moiety, the first and second charged moieties having opposite charges. In this embodiment, exchanging can include contacting one or more regions to be exchanged with a fluid including reagent promoting separation of the first and second charged moieties. In an embodiment, the reagent includes salt concentration of about 0.1 to about 2 M. In an embodiment, exchanging can include contacting one or more regions to be exchanged with a fluid including one or more building blocks and reagent promoting formation of ionic interactions.

In an embodiment, the functionalized lawn includes a first lipophilic moiety and the building block includes a second lipophilic moiety. In this embodiment, exchanging can include contacting one or more regions to be exchanged with a fluid including lipophilic reagent. In an embodiment, the lipophilic reagent includes organic solvent, surfactant, or mixture thereof. Suitable organic solvents and surfactants include those described hereinabove. In an embodiment, exchanging can include contacting one or more regions to be exchanged with a fluid including one or more building blocks and reagent promoting formation of hydrophobic interactions. In an embodiment, the reagent promoting formation of hydrophobic interactions includes water, or another nucleophilic or hydroxylic solvent.

In an embodiment, the method also includes determining the combinations of building blocks in one or more of the binding regions. The method can then include developing, based on the combinations determined, one or more developed sets of building blocks distinct from those in the one or more selected combinations of building blocks. This embodiment also includes exchanging into one or more of the regions one or more of the developed sets of building blocks. This embodiment can also include detecting binding of a test ligand to one or more of the exchanged regions and selecting one or more of the spots of the second heterogeneous molecular array as the artificial receptor. The artificial receptor can be a lead artificial receptor.

In an embodiment, this method includes varying the structure of the lead artificial receptor to increase binding speed or binding affinity of the test ligand. In an embodiment, the first set of building blocks includes a subset of a larger set of building blocks. In an embodiment, the first set of building blocks includes a subset of a larger set of building blocks, the second subset of building blocks defines a subset of the larger set of building blocks, and the first subset is not equivalent to the second subset. In an embodiment, the regions include 2, 3, or 4 building blocks.

In an embodiment, the method includes identifying the plurality of building blocks making up the artificial receptor; coupling the identified plurality of building blocks to a scaffold molecule; and evaluating the scaffold artificial receptor for binding of the test ligand. In an embodiment, coupling includes making a plurality of positional isomers of the building blocks on the scaffold; evaluating includes comparing the plurality of the scaffold positional isomer artificial receptors; and selecting one or more of the scaffold positional isomer artificial receptors as lead or working artificial receptor.

In an embodiment, the method includes applying the test ligand to one or more regions that function as controls for validating or evaluating binding to an artificial receptor. This embodiment can include employing a control region including no building block, only a single building block, only functionalized lawn, or a combination thereof.

Embodiments of methods including shuffling can also include exchanging building blocks onto or off of one or more regions. Embodiments of methods including exchanging can also include shuffling building blocks in one or more regions.

In an embodiment, the method includes shuffling before detecting. In an embodiment, the method includes detecting before shuffling. In an embodiment, the method includes shuffling, then detecting, then shuffling again. In an embodiment, the method includes contacting, then shuffling, then contacting again. In an embodiment, the method includes a combination thereof. In an embodiment, the method includes shuffling before detecting; detecting before shuffling; shuffling, then detecting, then shuffling again; contacting, then shuffling, then contacting again; or combinations thereof.

In an embodiment, this method includes shuffling before detecting. In an embodiment, the method includes detecting before shuffling. In an embodiment, the method includes shuffling, then detecting, then shuffling again. In an embodiment, the method includes contacting, then shuffling, then contacting again. In an embodiment, the method includes exchanging before detecting. In an embodiment, the method includes detecting before exchanging. In an embodiment, the method includes exchanging, then detecting, then exchanging again. In an embodiment, the method includes contacting, then exchanging, then contacting again. In an embodiment, the method includes shuffling before exchanging. In an embodiment, the method includes exchanging before shuffling. In an embodiment, the method includes combinations thereof.

In an embodiment, the method includes shuffling before detecting; detecting before shuffling; shuffling, then detecting, then shuffling again; contacting, then shuffling, then contacting again; exchanging before detecting; detecting before exchanging; exchanging, then detecting, then exchanging again; contacting, then exchanging, then contacting again; shuffling before exchanging; exchanging before shuffling; or combinations thereof.

In an embodiment, the method includes shuffling before detecting. In an embodiment, the method includes detecting before shuffling. In an embodiment, the method includes shuffling, then detecting, then shuffling again. In an embodiment, the method includes contacting, then shuffling, then contacting again. In an embodiment, the method includes exchanging before detecting. In an embodiment, the method includes detecting before exchanging. In an embodiment, the method includes exchanging, then detecting, then exchanging again. In an embodiment, the method includes contacting, then exchanging, then contacting again. In an embodiment, the method includes shuffling before exchanging. In an embodiment, the method includes exchanging before shuffling. In an embodiment, the method includes combinations thereof.

In an embodiment, the method includes shuffling before detecting; detecting before shuffling; shuffling, then detecting, then shuffling again; contacting, then shuffling, then contacting again; exchanging before detecting; detecting before exchanging; exchanging, then detecting, then exchanging again; contacting, then exchanging, then contacting again; shuffling before exchanging; exchanging before shuffling; or combinations thereof.

In an embodiment, the method includes exchanging before detecting. In an embodiment, the method includes detecting before exchanging. In an embodiment, the method includes exchanging, then detecting, then exchanging again. In an embodiment, the method includes contacting, then exchanging, then contacting again. In an embodiment, the method includes combinations thereof. In an embodiment, the method includes exchanging before detecting; detecting before exchanging; exchanging, then detecting, then exchanging again; contacting, then exchanging, then contacting again; or combinations thereof.

Detecting test ligand bound to a candidate artificial receptor can be accomplished using known methods for detecting binding to arrays on a slide or to coated tubes or wells. In an embodiment, the method employs test ligand labeled with a detectable label, such as a fluorophore or an enzyme that produces a detectable product. Alternatively, the method can employ an antibody (or other binding agent) specific for the test ligand and including a detectable label. The degree of labeling can be evaluated by evaluating the signal strength from the label. The amount of signal can be directly proportional to the amount of label and binding.

According to the present method, screening candidate artificial receptors against a test ligand can yield one or more lead artificial receptors. One or more lead artificial receptors can be a working artificial receptor. That is, the one or more lead artificial receptors can be useful for detecting the ligand of interest as is. The method can then employ the one or more artificial receptors as a working artificial receptor for monitoring or detecting the test ligand. Alternatively, the one or more lead artificial receptors can be employed in the method for developing a working artificial receptor. For example, the one or more lead artificial receptors can provide structural or other information useful for designing or screening for an improved lead artificial receptor or a working artificial receptor. Such designing or screening can include making and testing additional candidate artificial receptors including combinations of a subset of building blocks, a different set of building blocks, or a different number of building blocks.

In certain embodiments, the method of the present invention can employ a smaller number of spots formed by combinations of a subset of the total building blocks and/or smaller groups of the building blocks. For example, the present method can employ an array including the number of spots formed by combinations of 81 building blocks in groups of 2 and/or 3. Then a smaller number of building blocks indicated by test compound binding, for example 36 building blocks, can be tested in a microarray with spots including larger groups, for example 4, of the building blocks.

Test Ligands

The test ligand can be any ligand for which binding to an array or surface can be detected. The test ligand can be a pure compound, a mixture, or a “dirty” mixture containing a natural product or pollutant. Such dirty mixtures can be tissue homogenate, biological fluid, soil sample, water sample, or the like.

Test ligands include prostate specific antigen, other cancer markers, insulin, warfarin, other anti-coagulants, cocaine, other drugs-of-abuse, markers for E. coli, markers for Salmonella sp., markers for other food-borne toxins, food-borne toxins, markers for Smallpox virus, markers for anthrax, markers for other possible infectious agents, pharmaceuticals and medicines, pollutants and chemicals in hazardous waste, nerve agents, other toxic chemical agents, markers of disease, pharmaceuticals, pollutants, biologically important cations (e.g., potassium or calcium ion), peptides, carbohydrates, enzymes, bacteria, viruses, mixtures thereof, and the like. In certain embodiments, the test ligand can be at least one of small organic molecules, inorganic/organic complexes, metal ion, mixture of proteins, protein, nucleic acid, mixture of nucleic acids, mixtures thereof, and the like.

### EXAMPLES

### Example 1

### Synthesis of Building Blocks

Selected building blocks representative of the alkyl-aromatic-polar span of the an embodiment of the building blocks were synthesized and demonstrated effectiveness of these building blocks for making candidate artificial receptors. These building blocks were made on a framework that can be represented by tyrosine and included numerous recognition element pairs. These recognition element pairs were selected along the diagonal of Table 2, and include enough of the range from alkyl, to aromatic, to polar to represent a significant degree of the interactions and functional groups of the full set of 81 such building blocks.

Synthesis

Building block synthesis employed a general procedure outlined in Scheme 2, which specifically illustrates synthesis of a building block on a tyrosine framework with recognition element pair A4B4. This general procedure was employed for synthesis of building blocks including TyrA1B1 [1-1], TyrA2B2, TyrA2B4, TyrA2B6, TyrA2B8, TyrA4B2, TyrA4B4, TyrA4B6, TyrA4B8, TyrA6B2, TyrA6B4, TyrA6B6, TyrA6B8, TyrA8B2, TyrA8B4, TyrA8B6, TyrA8B8, and TyrA9B9, respectively. 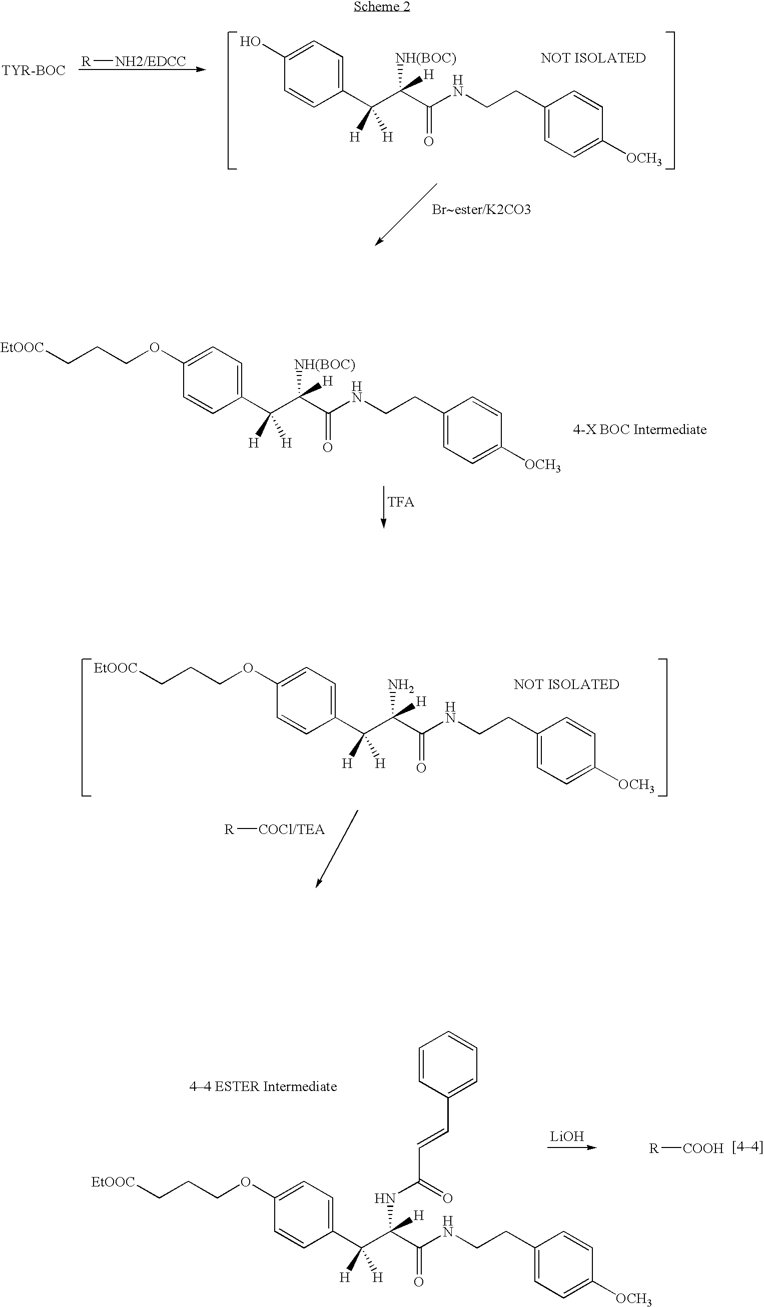
Results

Synthesis of the desired building blocks proved to be generally straightforward. These syntheses illustrate the relative simplicity of preparing the building blocks with 2 recognition elements having different structural characteristics or structures (e.g. A4B2, A6B3, etc.) once the building blocks with corresponding recognition elements (e.g. A2B2, A4B4, etc) have been prepared via their X BOC intermediate.

The conversion of one of these building blocks to a building block with a lipophilic linker can be accomplished by reacting the activated building block with, for example, dodecyl amine.

### Example 2

### Preparation and Evaluation of Microarrays of Candidate Artificial Receptors

Microarrays of candidate artificial receptors were made and evaluated for binding several protein ligands. The results obtained demonstrate the 1) the simplicity with which microarrays of candidate artificial receptors can be prepared, 2) binding affinity and binding pattern reproducibility, 3) significantly improved binding for building block heterogeneous receptor environments when compared to the respective homogeneous controls, and 4) ligand distinctive binding patterns (e.g., working receptor complexes).

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6. The abbreviation for the building block including a linker, a tyrosine framework, and recognition elements AxBy is TyrAxBy.

Microarrays for the evaluation of the 130 n=2 and n=3, and for evaluation of the 273 n=2, n=3, and n=4, candidate receptor environments were prepared as follows by modifications of known methods. Briefly: Amine modified (amine “lawn”; SuperAmine Microarray plates) microarray plates were purchased from Telechem Inc., Sunnyvale, Calif. (www.arrayit.com). These plates were manufactured specifically for microarray preparation and had a nominal amine load of 2-4 amines per square nm according to the manufacturer. The CAM microarrays were prepared using a pin microarray spotter instrument from Telechem Inc. (SpotBot™ Arrayer) typically with 200 um diameter spotting pins from Telechem Inc. (Stealth Micro Spotting Pins, SMP6) and 400-420 um spot spacing.

The 9 building blocks were activated in aqueous dimethylformamide (DMF) solution as described above. For preparing the 384-well feed plate, the activated building block solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). A separate series of controls were prepared by aliquotting 10 μl of building block with either 10 μl or 20 μl of the activated [1-1] solution. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was also used to rinse the microarrays on completion of the SpotBot™ printing run. The plates were given a final rinse with deionized (DI) water, dried using a stream of compressed air, and stored at room temperature.

Certain of the microarrays were further modified by reacting the remaining amines with succinic anhydride to form a carboxylate lawn in place of the amine lawn.

The following test ligands and labels were used in these experiments:
1) r-Phycoerythrin, a commercially available and intrinsically fluorescent protein with a FW of 2,000,000.
2) Ovalbumin labeled with the Alexa™ fluorophore (Molecular Probes Inc., Eugene, Oreg.).
3) BSA, bovine serum albumin, labeled with activated Rhodamine (Pierce Chemical, Rockford, Ill.) using the known activated carboxylprotocol. BSA has a FW of 68,000; the material used for this study had ca. 1.0 rhodamine per BSA.
4) Horseradish peroxidase (HRP) modified with extra amines and labeled as the acetamide derivative or with a 2,3,7,8-tetrachlorodibenzodixoin derivative were available through known methods. Fluorescence detection of these HRP conjugates was based on the Alexa 647-tyramide kit available from Molecular Probes, Eugene, Oreg.
5) Cholera toxin.

Microarray incubation and analysis was conducted as follows: For test ligand incubation with the microarrays, solutions (e.g. 500 μl) of the target proteins in PBS-T (PBS with 20 μl/L of Tween-20) at typical concentrations of 10, 1.0 and 0.1 μg/ml were placed onto the surface of a microarray and allowed to react for, e.g., 30 minutes. The microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Results

The CARA™: Combinatorial Artificial Receptor Array™ concept has been demonstrated using a microarray format. A CARA microarray based on N=9 building blocks was prepared and evaluated for binding to several protein and substituted protein ligands. This microarray included 144 candidate receptors (18 n=1 controls plus 6 blanks; 36 n=2 candidate receptors; 84 n=3 candidate receptors). This microarray demonstrated: 1) the simplicity of CARA microarray preparation, 2) binding affinity and binding pattern reproducibility, 3) significantly improved binding for building block heterogeneous receptor environments when compared to the respective homogeneous controls, and 4) ligand distinctive binding patterns.

Reading the Arrays

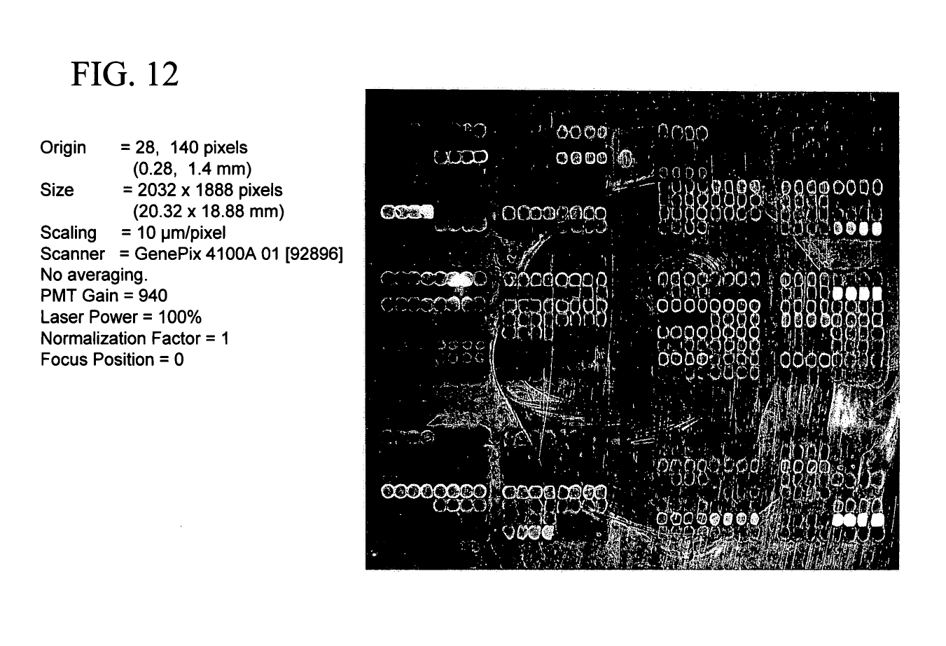

A typical false color/gray scale image of a microarray that was incubated with 2.0 μg/ml r-phycoerythrin is shown in FIG. 12. This image illustrates that the processes of both preparing the microarray and probing it with a protein test ligand produced the expected range of binding as seen in the visual range of relative fluorescence from dark to bright spots.

The starting point in analysis of the data was to take the integrated fluorescence units data for the array of spots and normalize to the observed value for the [1-1] building block control. Subsequent analysis included mean, standard deviation and coefficient of variation calculations. Additionally, control values for homogeneous building blocks were obtained from the building block plus [1-1] data.

First Set of Experiments

The following protein ligands were evaluated for binding to the candidate artificial receptors in the microarray. The resulting Fluorescence Units versus candidate receptor environment data is presented in both a 2D format where the candidate receptors are placed along the X-axis and the Fluorescence Units are shown on the Y-axis and a 3D format where the Candidate Receptors are placed in an X-Y format and the Fluorescence Units are shown on the Z-axis. A key for the composition of each spot was developed (not shown). A key for the building blocks in each of the 2D and 3D representations of the results was also developed (not shown). The data presented are for 1-2 μg/ml protein concentrations.

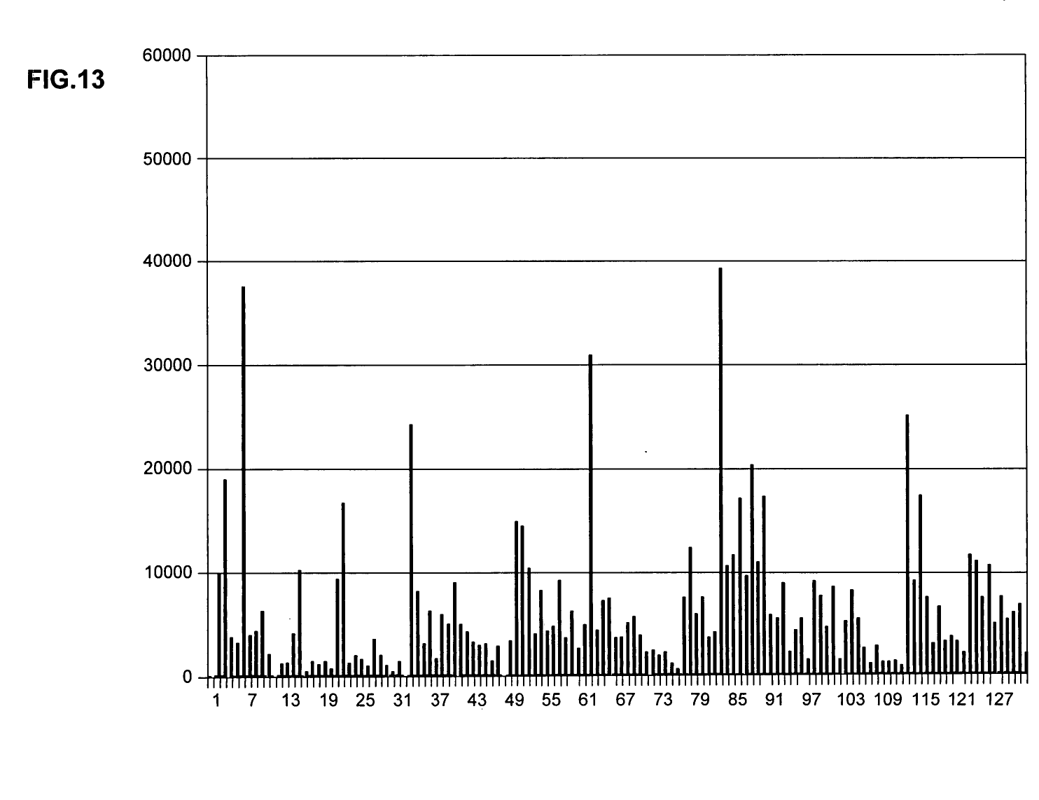

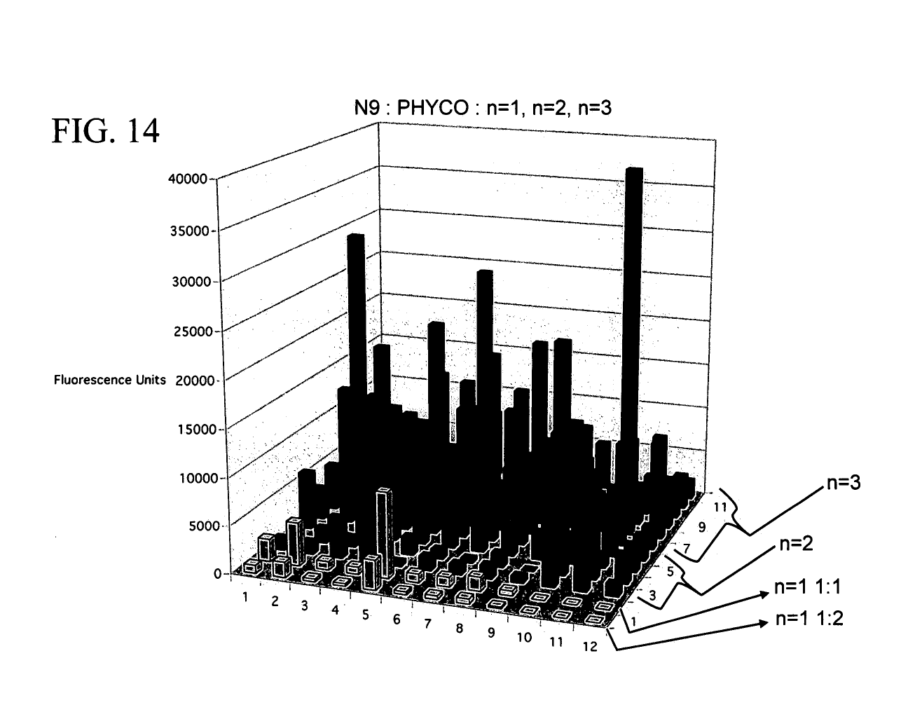

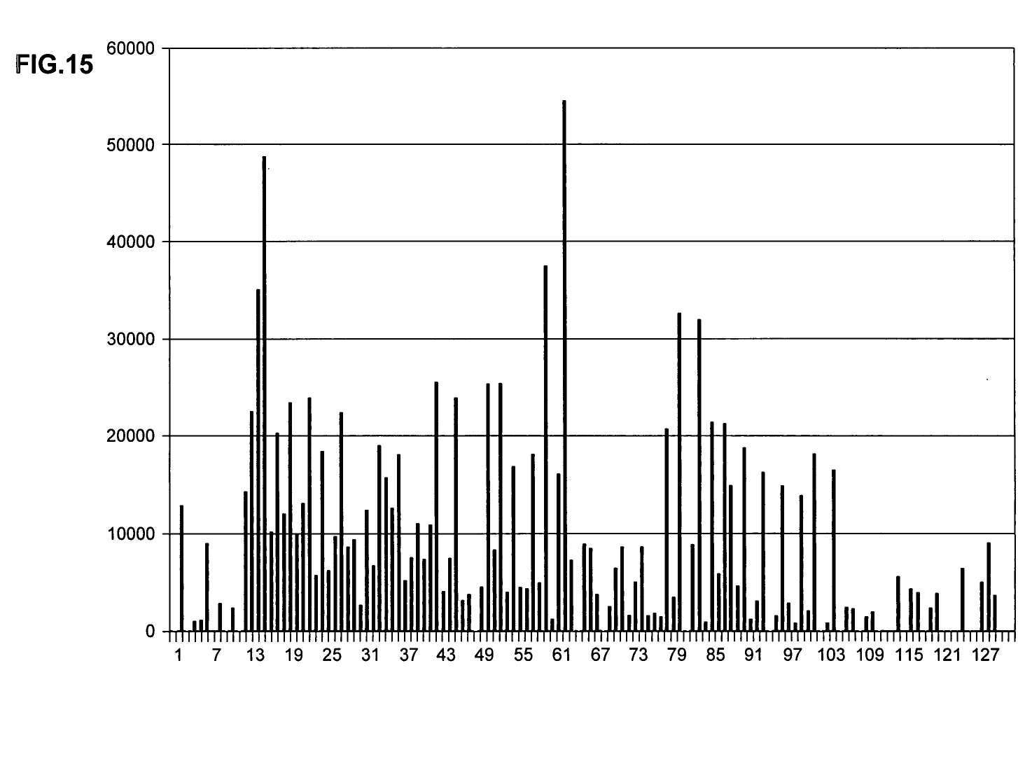

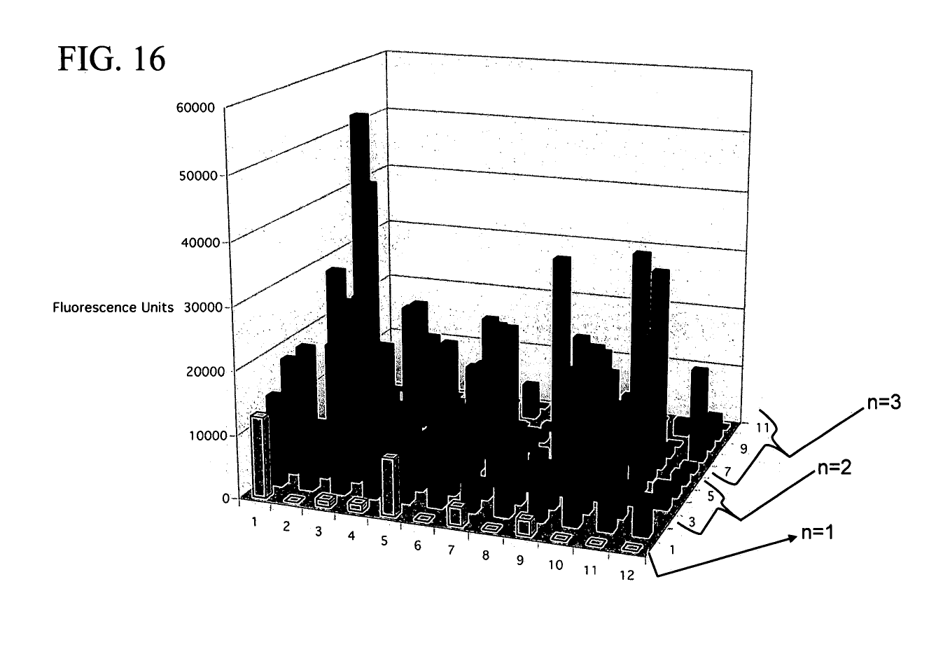

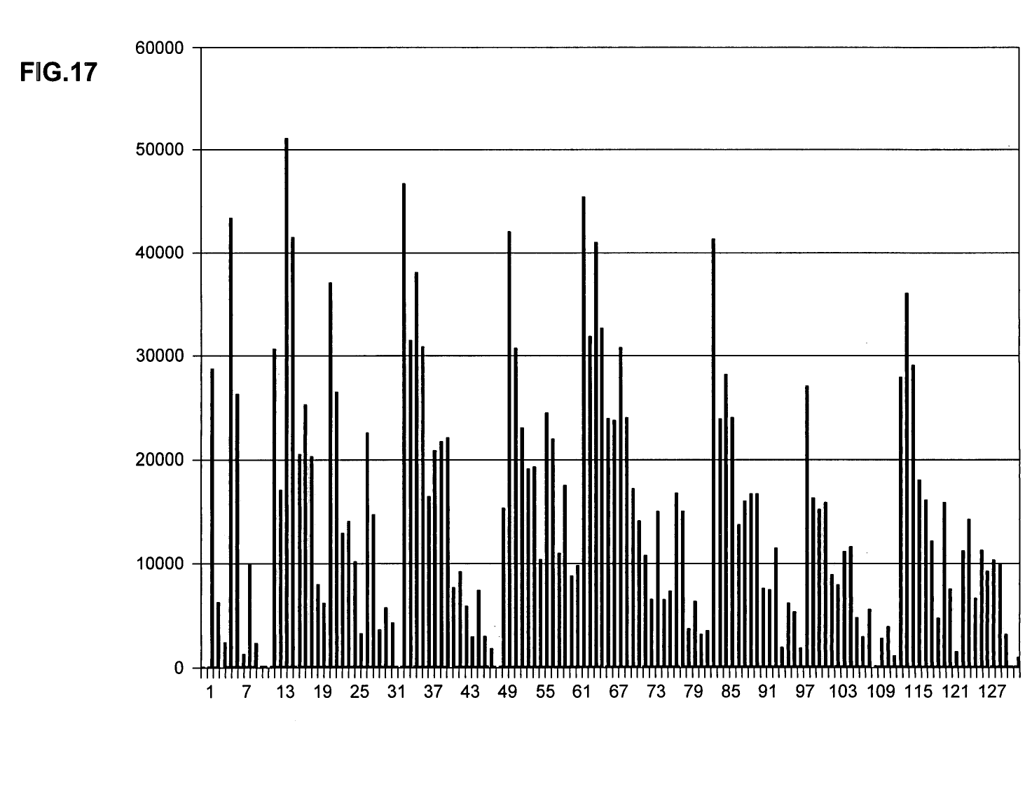

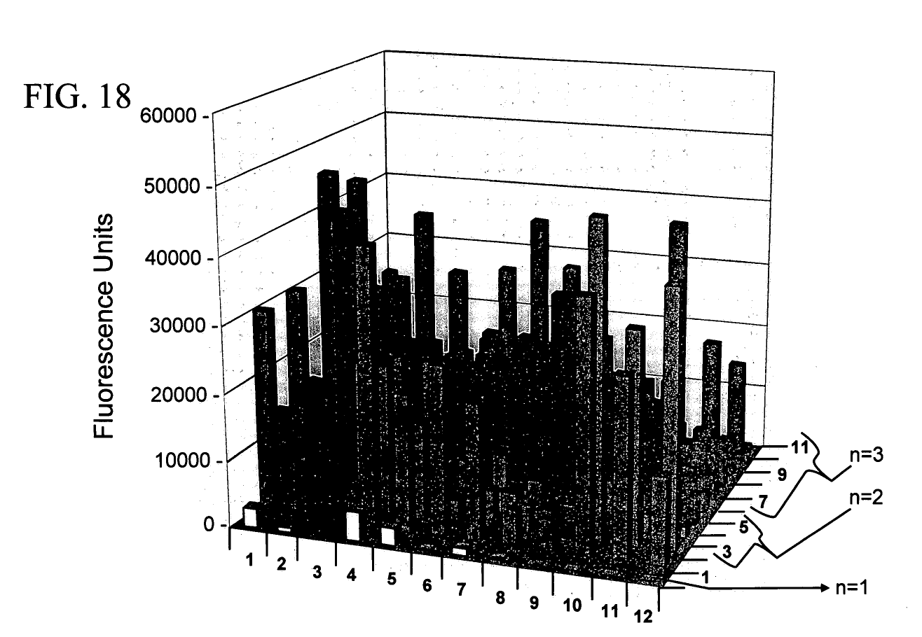

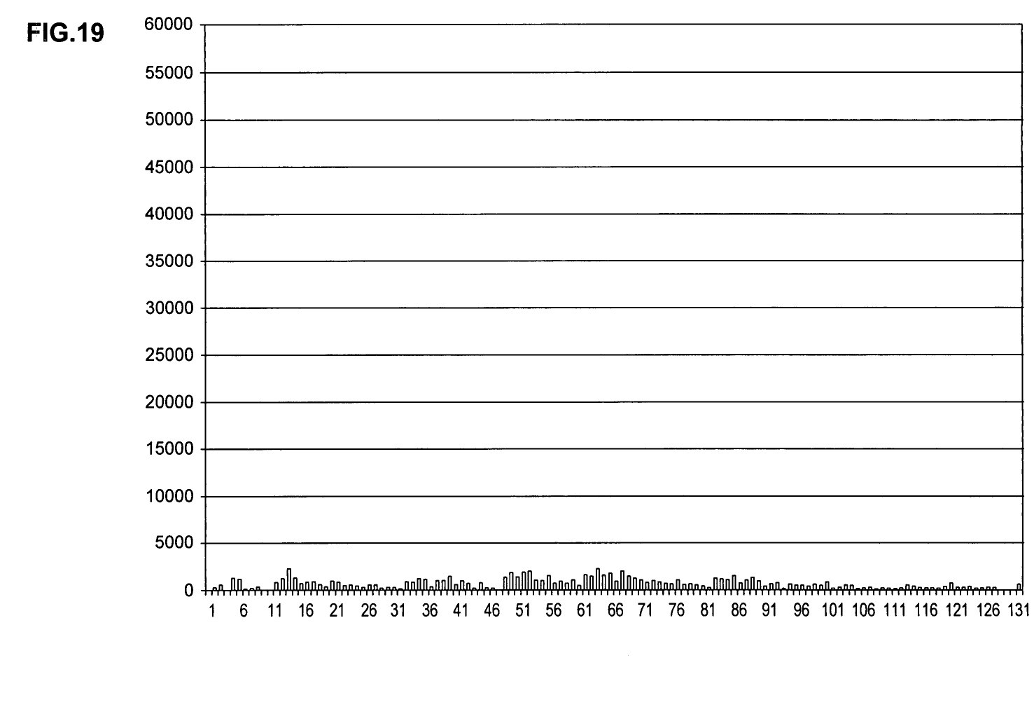

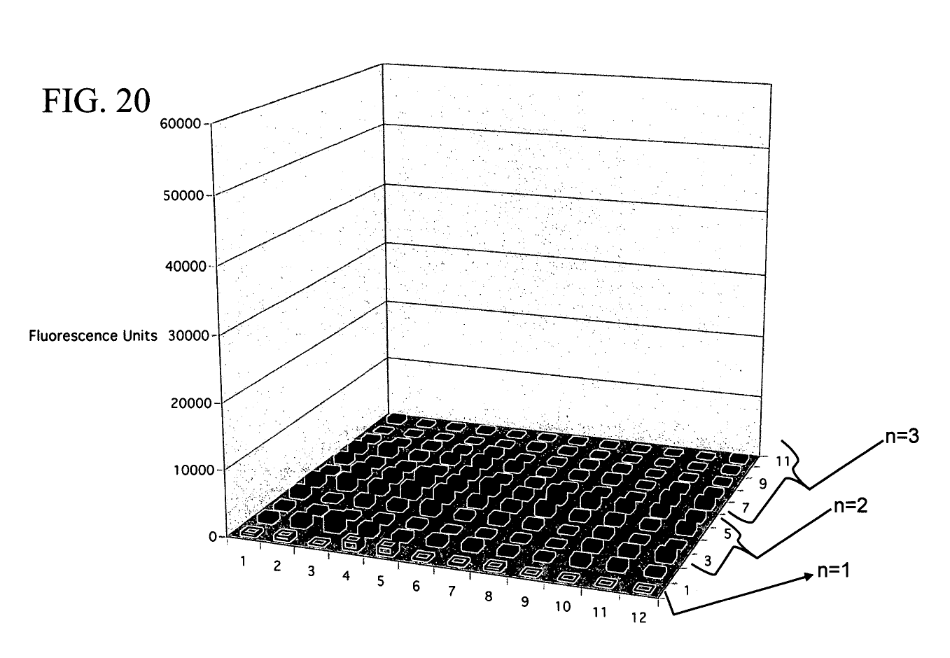

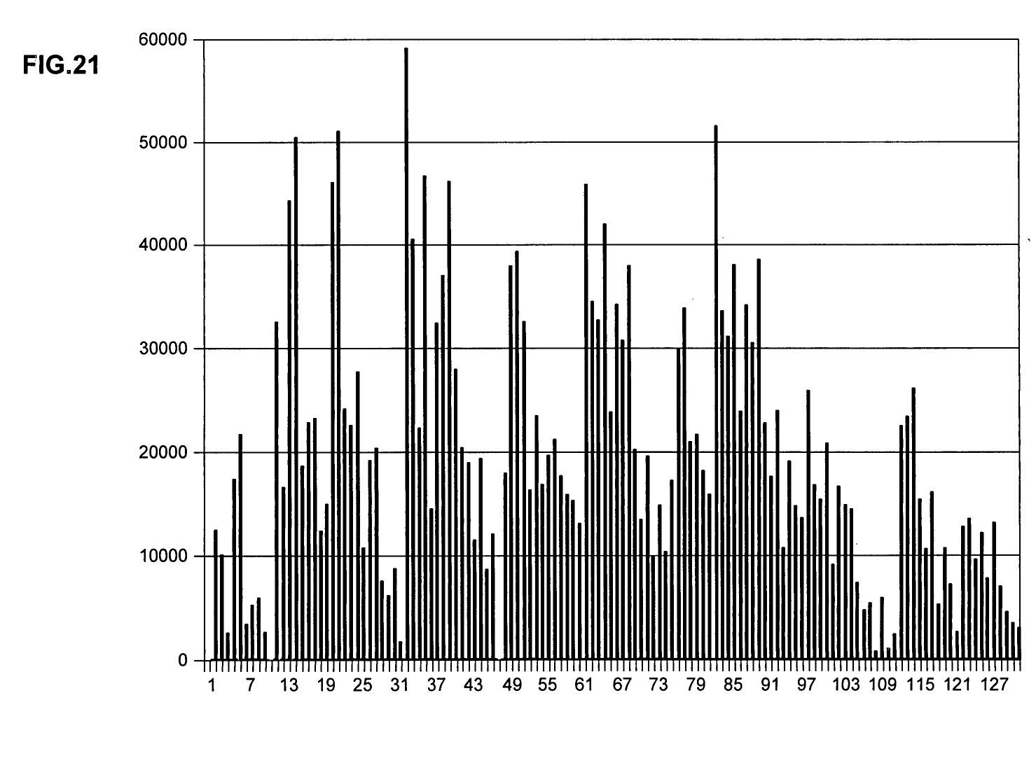

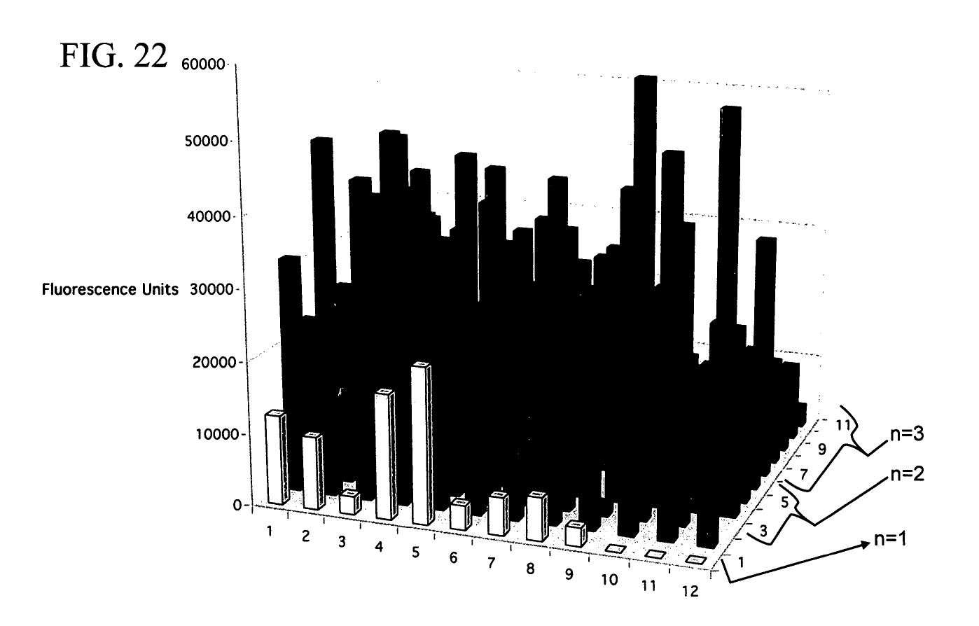

FIGS. 13 and 14 illustrate binding data for r-phycoerythrin (intrinsic fluorescence). FIGS. 15 and 16 illustrate binding data for ovalbumin (commercially available with fluorescence label). FIGS. 17 and 18 illustrate binding data for bovine serum albumin (labeled with rhodamine). FIGS. 19 and 20 illustrate binding data for HRP—NH—Ac (fluorescent tyramide read-out). FIGS. 21 and 22 illustrate binding data for HRP—NH-TCDD (fluorescent tyramide read-out).

These results demonstrate not only the application of the CARA microarray to candidate artificial receptor evaluation but also a few of the many read-out methods (e.g. intrinsic fluorescence, fluorescently labeled, in situ fluorescence labeling) which can be utilized for high throughput candidate receptor evaluation.

The evaluation of candidate receptors benefits from reproducibility. The following results demonstrate that the present microarrays provided reproducible ligand binding.

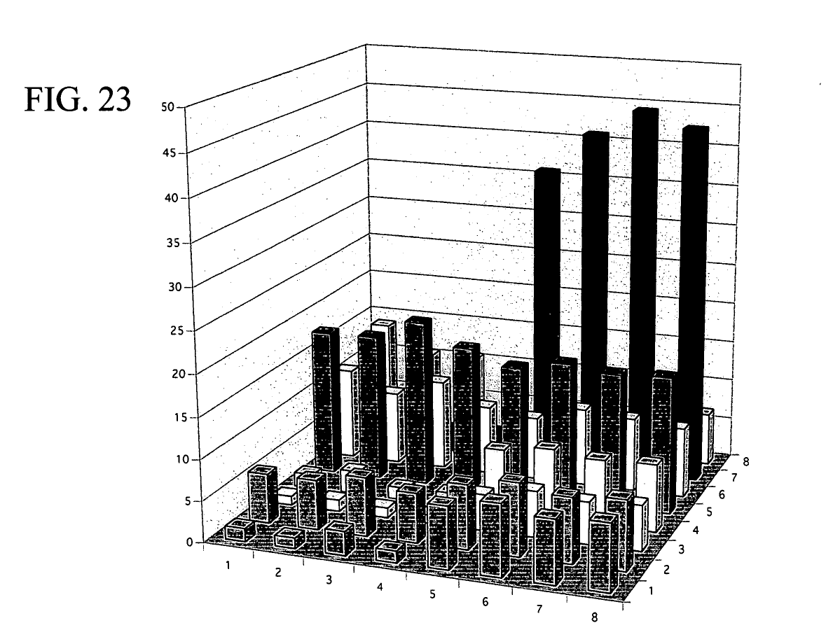

The microarrays were printed with each combination of building blocks spotted in quadruplicate. Visual inspection of a direct plot (FIG. 23) of the raw fluorescence data (from the run illustrated in FIG. 12) for one block of binding data obtained for r-phycoerythrin demonstrates that the candidate receptor environment “spots” showed reproducible binding to the test ligand. Further analysis of the r-phycoerythrin data (FIG. 12) led to only 9 out of 768 spots (1.2%) being deleted as outliers. Analysis of the r-phycoerythrin quadruplicate data for the entire array gives a mean standard deviation for each experimental quadruplicate set of 938 fluorescence units, with a mean coefficient of variation of 19.8%.

Although these values are acceptable, a more realistic comparison employed the standard deviation and coefficient of variation of the more strongly bound, more fluorescent receptors. The overall mean standard deviation unrealistically inflates the coefficient of variation for the weakly bound, less fluorescent receptors. The coefficient of variation for the 19 receptors with greater than 10,000 Fluorescent Units of bound target is 11.1%, which is well within the range required to produce meaningful binding data.

One goal of the CARA approach is the facile preparation of a significant number of candidate receptors through combinations of structurally simple building blocks. The following results establish that both the individual building blocks and combinations of building blocks have a significant, positive effect on test ligand binding.

The binding data illustrated in FIGS. 54-22 demonstrate that heterogeneous combinations of building blocks (n=2, n=3) are dramatically superior candidate receptors made from a single building block (n=1). For example, FIG. 14 illustrate both the diversity of binding observed for n=2, n=3 candidate receptors with fluorescent units ranging from 0 to ca. 40,000. These data also illustrate and the ca. 10-fold improvement in binding affinity obtained upon going from the homogeneous (n=1) to heterogeneous (n=2, n=3) receptor environments.

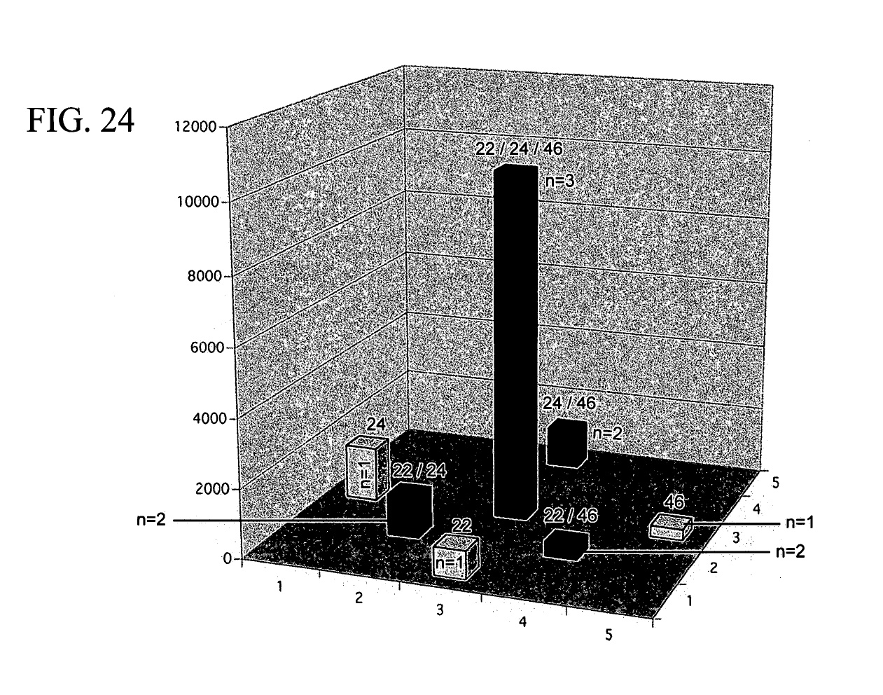

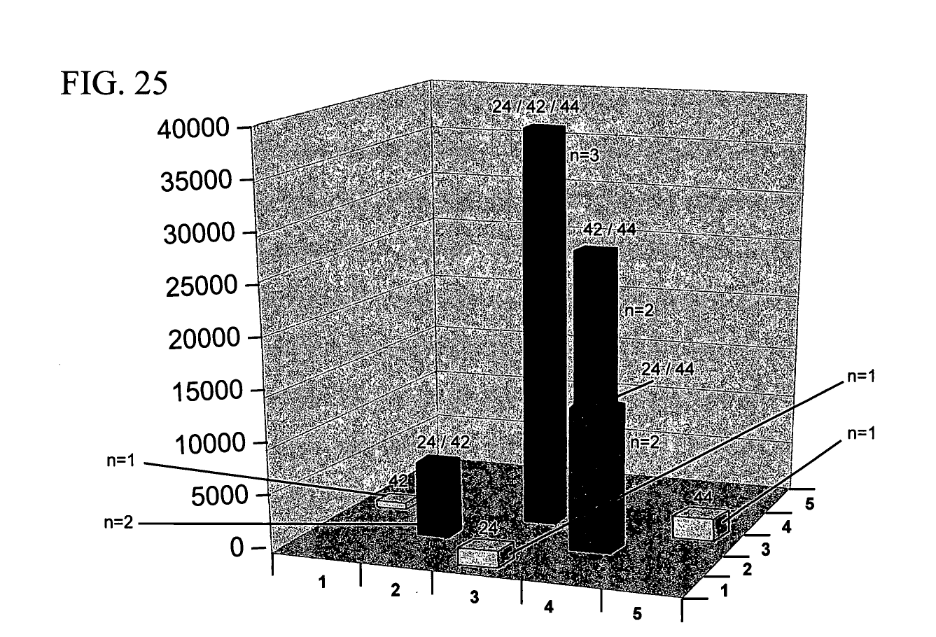

The effect of heterogeneous building blocks is most easily observed by comparing selected n=3 receptor environments candidate receptors including 1 or 2 of those building blocks (their n=2 and n=1 subsets). FIGS. 24 and 25 illustrate this comparison for two different n=3 receptor environments using the r-phycoerythrin data. In these examples, it is clear that progression from the homogeneous system (n=1) to the heterogeneous systems (n=2, n=3) produces significantly enhanced binding.

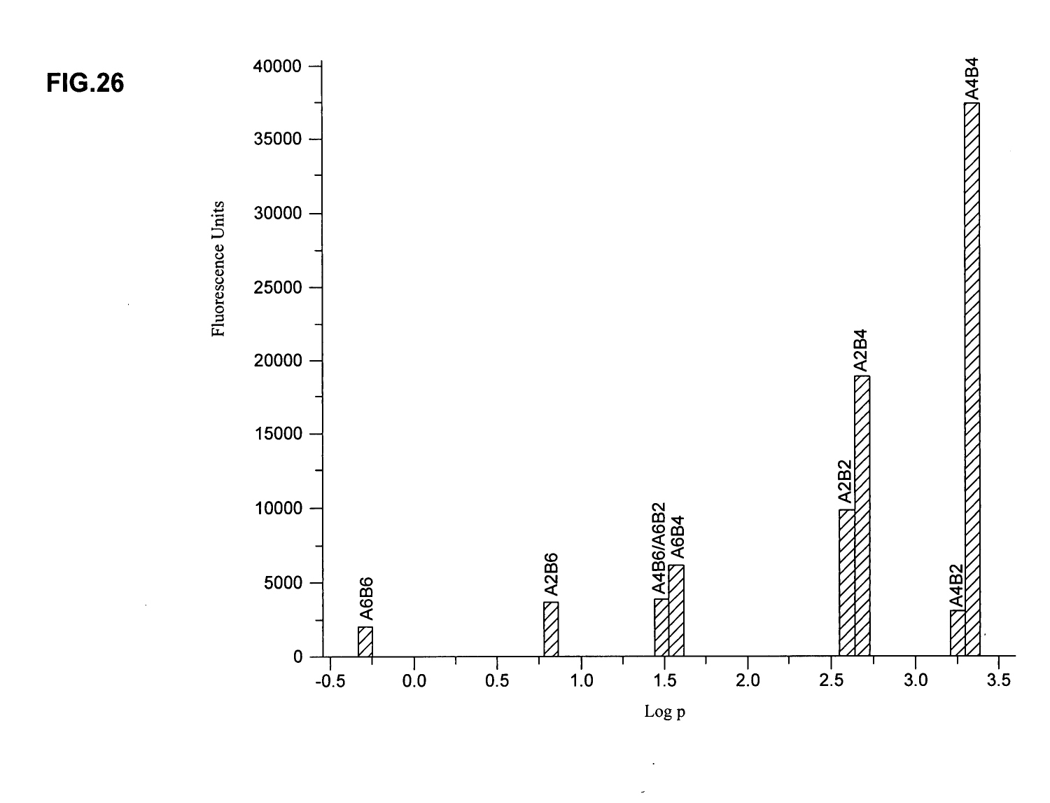

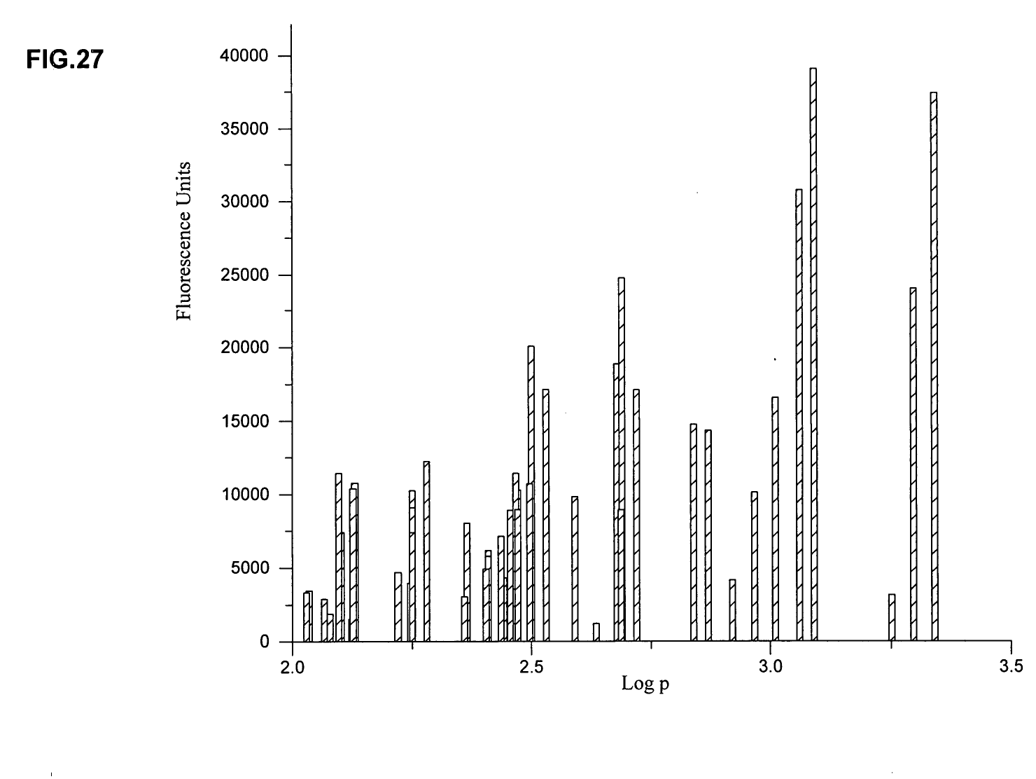

Although van der Waals interactions are an important part of molecular recognition, it is important to establish that the observed binding is not a simple case of hydrophobic/hydrophilic partitioning. That is, that the observed binding was the result of specific interactions between the individual building blocks and the target The simplest way to evaluate the effects of hydrophobicity and hydrophilicity is to compare building block logP value with observed binding. LogP is a known and accepted measure of lipophilicity, which can be measured or calculated by known methods for each of the building blocks. FIGS. 26 and 27 establish that the observed target binding, as measured by fluorescence units, is not directly proportional to building block logP. The plots in FIGS. 26 and 27 illustrate a non-linear relationship between binding (fluorescence units) and building block logP.

One advantage of the present methods and arrays is that the ability to screen large numbers of candidate receptor environments will lead to a combination of useful target affinities and to significant target binding diversity. High target affinity is useful for specific target binding, isolation, etc. while binding diversity can provide multiplexed target detection systems. This example employed a relatively small number of building blocks to produce ca. 120 binding environments. The following analysis of the present data clearly demonstrates that even a relatively small number of binding environments can produce diverse and useful artificial receptors.

The target binding experiments performed for this study used protein concentrations including 0.1 to 10 μg/ml. Considering the BSA data as representative, it is clear that some of the receptor environments readily bound 1.0 ug/ml BSA concentrations near the saturation values for fluorescence units (see, e.g., FIG. 18). Based on these data and the formula weight of 68,000 for BSA, several of the receptor environments readily bind BSA at ca. 15 picomole/ml or 15 nanomolar concentrations. Additional experiments using lower concentrations of protein (data not shown) indicate that, even with a small selection of candidate receptor environments, femptomole/ml or picomolar detection limits have been attained.

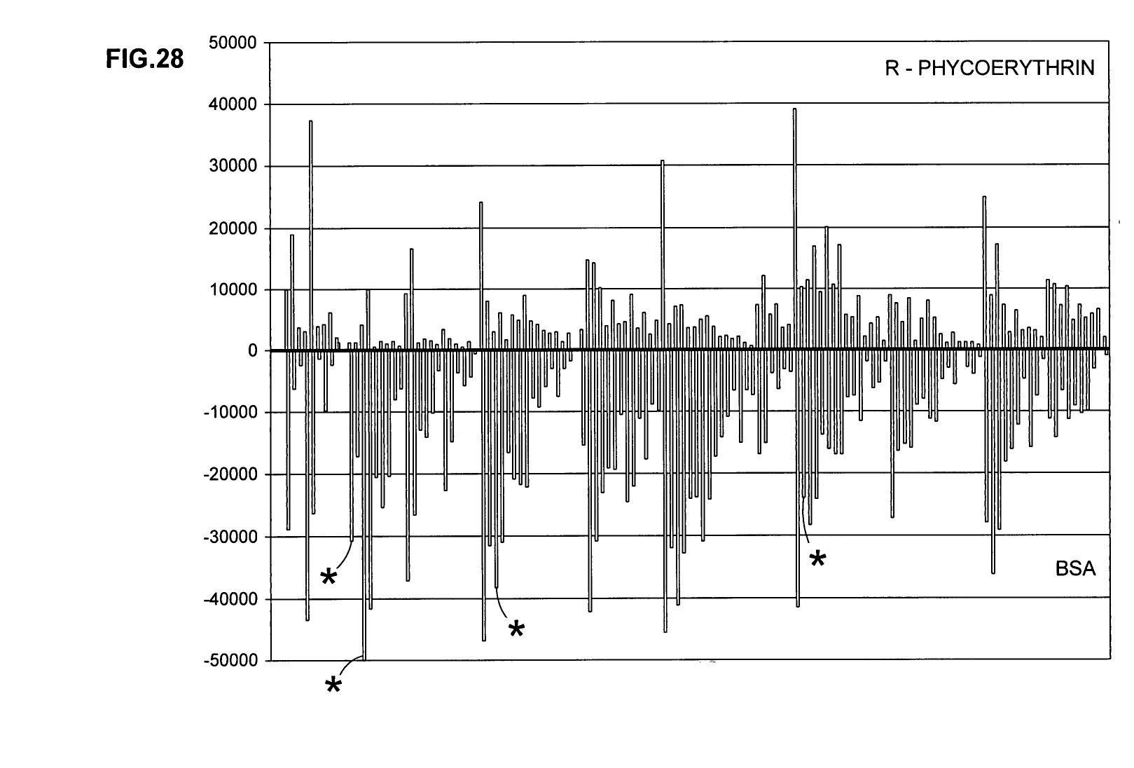

One goal of artificial receptor development is the specific recognition of a particular target. FIG. 28 compares the observed binding for r-phycoerythrin and BSA. Comparison of the overall binding pattern indicates some general similarities. However, comparison of specific features of binding for each receptor environment demonstrates that the two targets have distinctive recognition features as indicated by the (*) in FIG. 28.

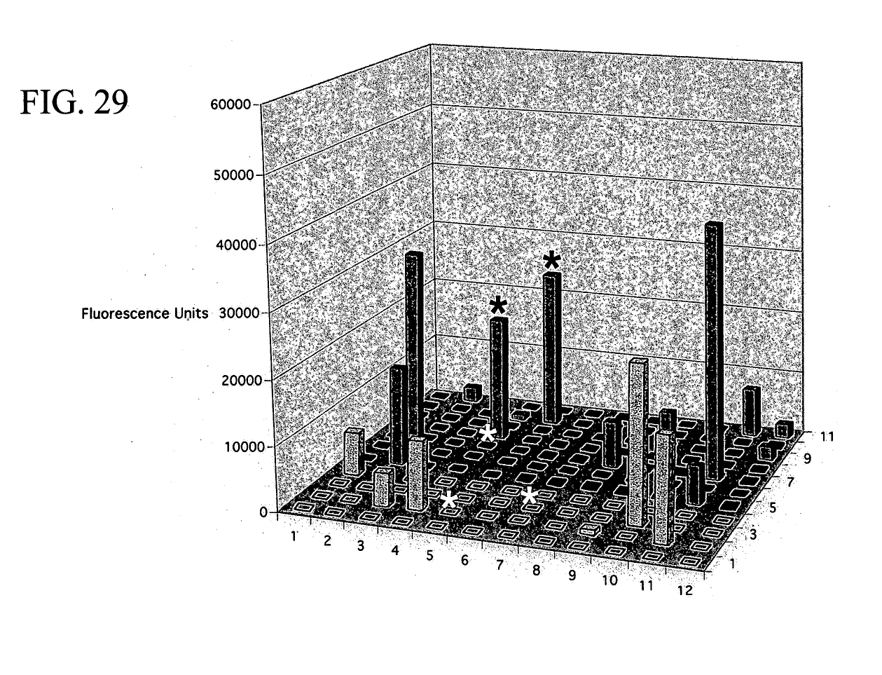

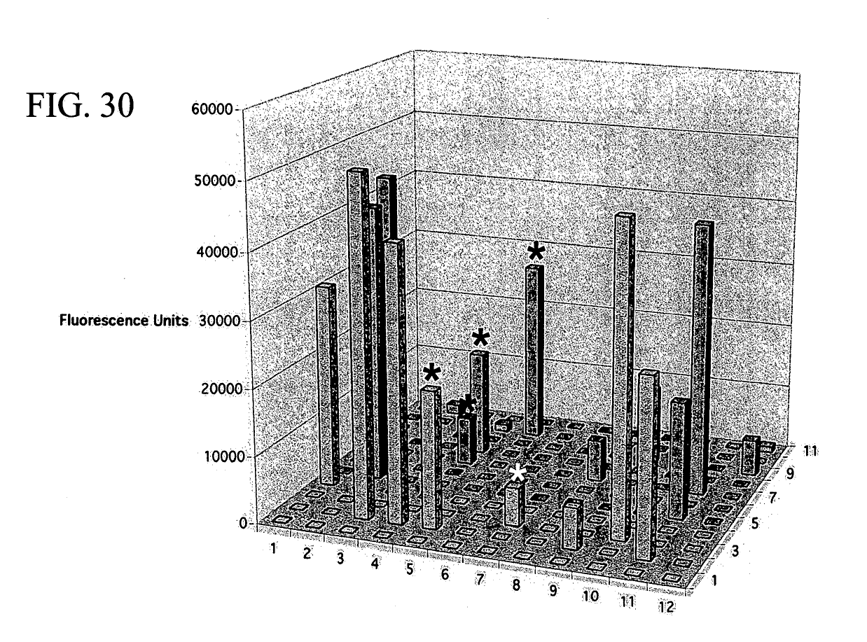

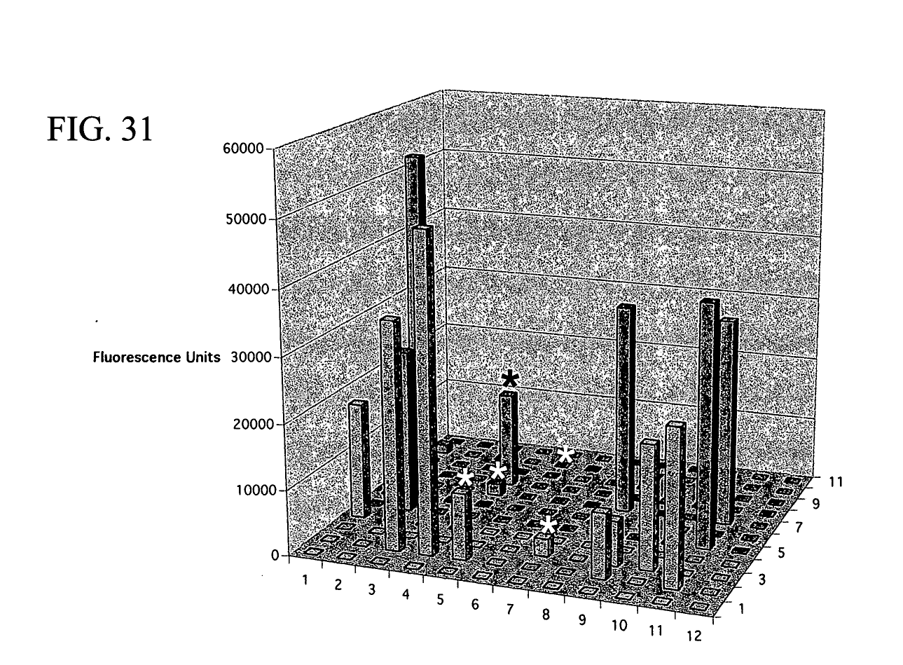

One goal of artificial receptor development is to develop receptors which can be used for the multiplexed detection of specific targets. Comparison of the r-phycoerythrin, BSA and ovalbumin data from this study (FIGS. 14, 16, 18) were used to select representative artificial receptors for each target. FIGS. 29, 30 and 31 employ data obtained in the present example to illustrate identification of each of these three targets by their distinctive binding patterns.

### CONCLUSIONS

The optimum receptor for a particular target requires molecular recognition which is greater than the expected sum of the individual hydrophilic, hydrophobic, ionic, etc. interactions. Thus, the identification of an optimum (specific, sensitive) artificial receptor from the limited pool of candidate receptors explored in this prototype study, was not expected and not likely. Rather, the goal was to demonstrate that all of the key components of the CARA: Combinatorial Artificial Receptor Array concept could be assembled to form a functional receptor microarray. This goal has been successfully demonstrated.

This study has conclusively established that CARA microarrays can be readily prepared and that target binding to the candidate receptor environments can be used to identify artificial receptors and test ligands. In addition, these results demonstrate that there is significant binding enhancement for the building block heterogeneous (n=2, n=3, or n=4) candidate receptors when compared to their homogeneous (n=1) counterparts. When combined with the binding pattern recognition results and the demonstrated importance of both the heterogeneous receptor elements and heterogeneous building blocks, these results clearly demonstrate the significance of the CARA Candidate Artificial Receptor->Lead Artificial Receptor->Working Artificial Receptor strategy.

### Example 3

### Preparation and Evaluation of Microarrays of Candidate Artificial Receptors Including Reversibly Immobilized Building Blocks

Microarrays of candidate artificial receptors including building blocks immobilized through van der Waals interactions were made and evaluated for binding of a protein ligand. The evaluation was conducted at several temperatures, above and below a phase transition temperature for the lawn (vide infra).

Materials and Methods

Building blocks 2-2,2-4, 2-6,4-2, 4-4,4-6, 6-2,6-4, 6-6 where prepared as described in Example 1. The C12 amide was prepared using the previously described carbodiimide activation of the carboxyl followed by addition of dodecylamine.

Amino lawn microarray plates (Telechem) were modified to produce the C18 lawn by reaction of stearoyl chloride (Aldrich Chemical Co.) in A) dimethylformamide/PEG 400 solution (90:10, v/v, PEG 400 is polyethylene glycol average MW 400 (Aldrich Chemical Co.) or B) methylene chloride/TEA solution (100 ml methylene chloride, 200 ul triethylamine) using the lawn modification procedures generally described in Example 2.

The C18 lawn plates where printed using the SpotBot standard procedure as described in Example 2. The building blocks were in printing solutions prepared by solution of ca. 10 mg of each building block in 300 ul of methylene chloride and 100 ul methanol. To this stock was added 900 ul of dimethylformamide and 100 ul of PEG 400. The 36 combinations of the 9 building blocks taken two at a time (N9:n2, 36 combinations) where prepared in a 384-well microwell plate which was then used in the SpotBot to print the microarray in quadruplicate. A random selection of the print positions contained only print solution.

The selected microarray was incubated with a 1.0 μg/ml solution of the probe protein (e.g. fluorescently labeled cholera toxin B) using the following variables: the microarray was washed with methylene chloride, ethanol and water to create a control plate, the microarray was incubated at 4° C., 23° C., or 44° C. After incubation, the plate(s) were rinsed with water, dried and scanned (AXON 4100A). Data analysis was as described in Example 2.

Results

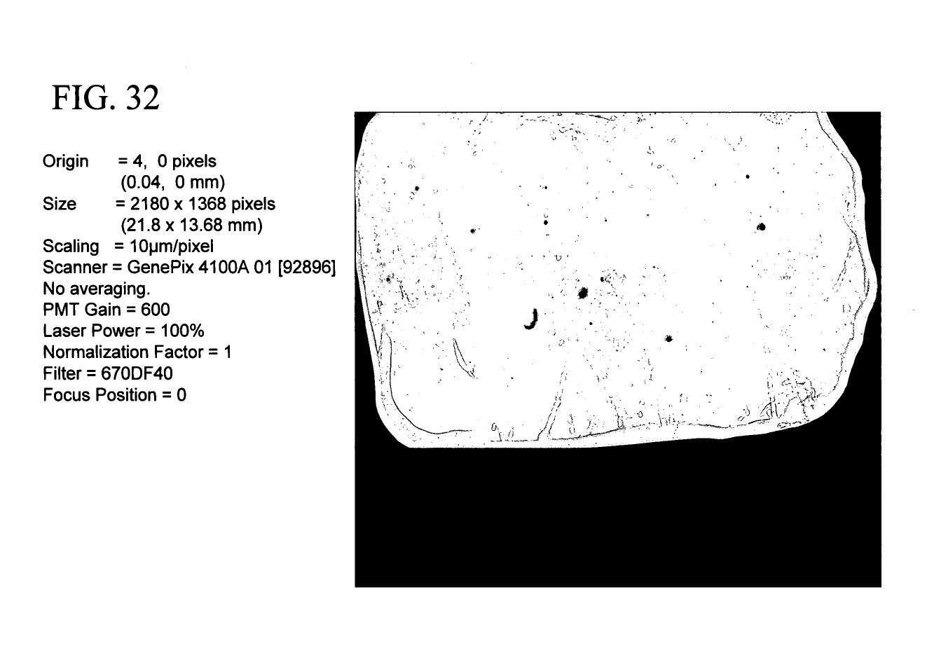

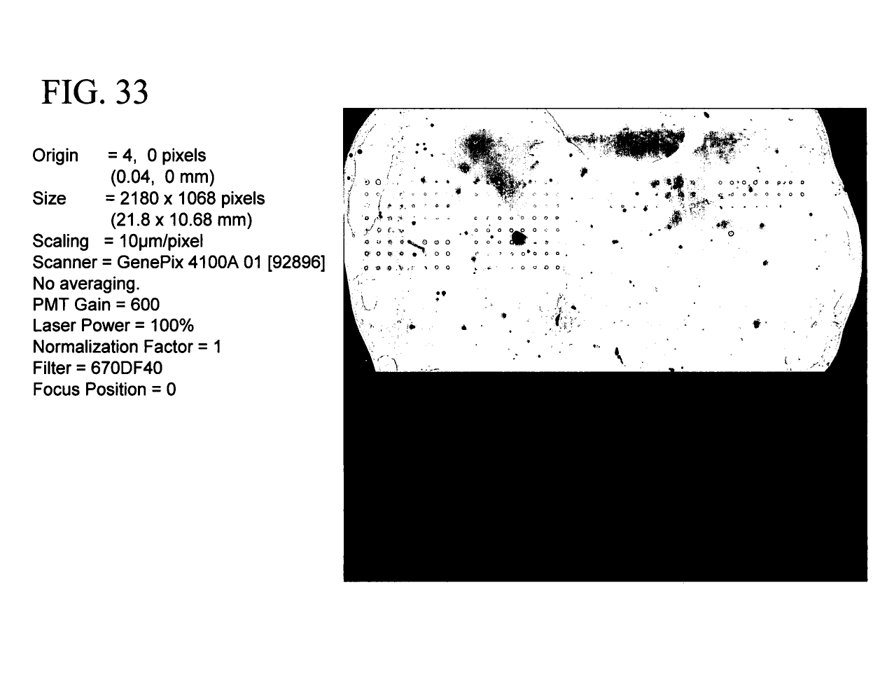

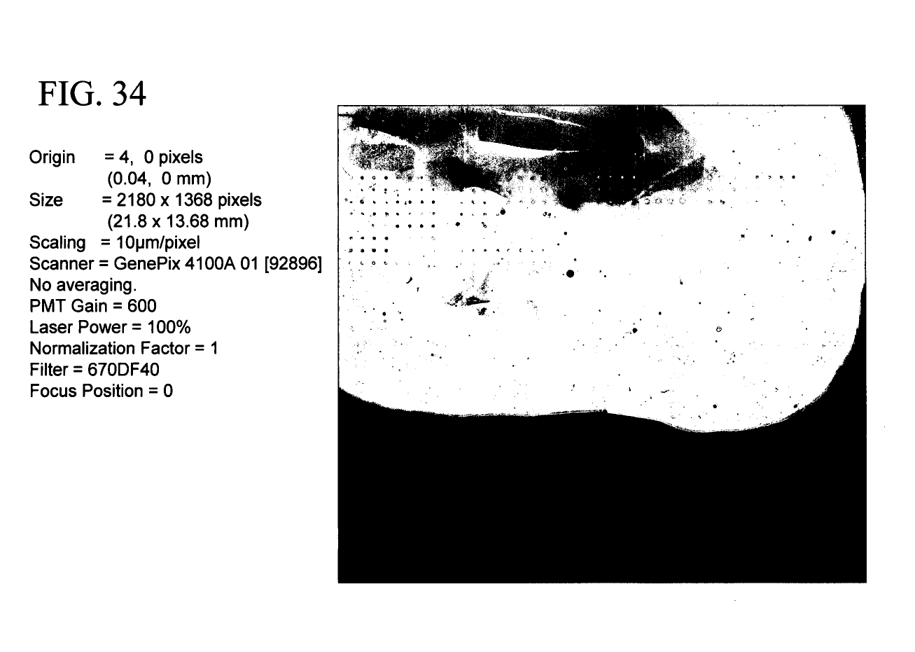

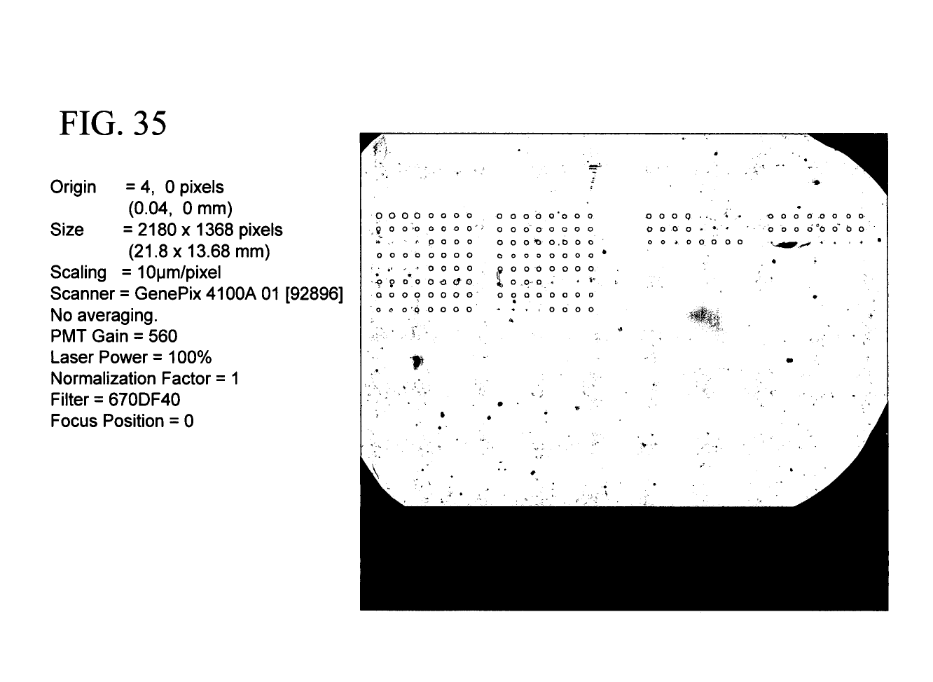

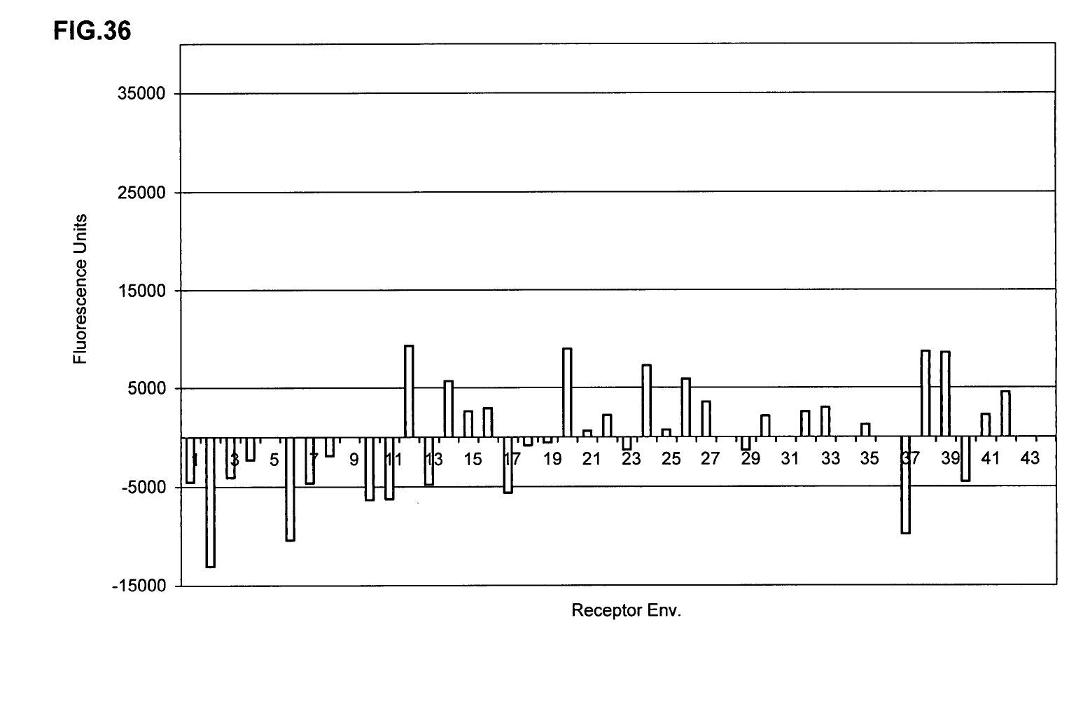

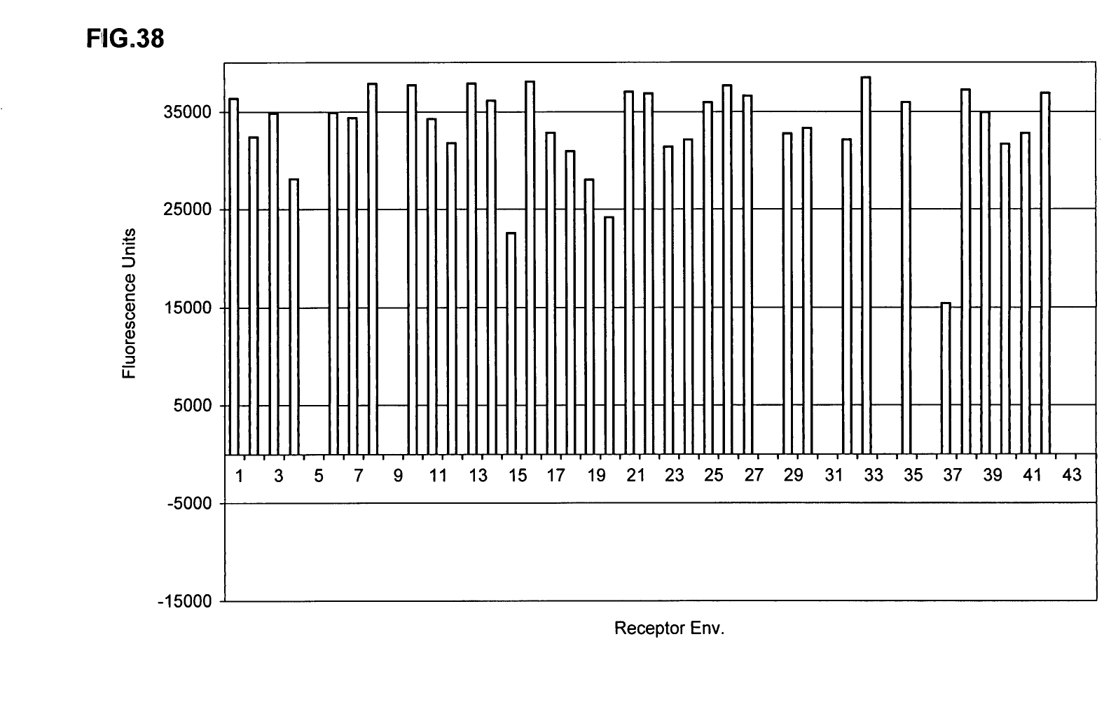

A control array from which the building blocks had been removed by washing with organic solvent did not bind cholera toxin (FIG. 32). FIGS. 33-35 illustrate fluorescence signals from arrays printed identically, but incubated with cholera toxin at 4° C., 23° C., or 44° C., respectively. Spots of fluorescence can be seen in each array, with very pronounced spots produced by incubation at 44° C. The fluorescence values for the spots in each of these three arrays are shown in FIGS. 36-38. Fluorescence signal generally increases with temperature, with many nearly equally large signals observed after incubation at 44° C. Linear increases with temperature can reflect expected improvements in binding with temperature. Nonlinear increases reflect rearrangement of the building blocks on the surface to achieve improved binding, which occurred above the phase transition for the lipid surface (vide infra).

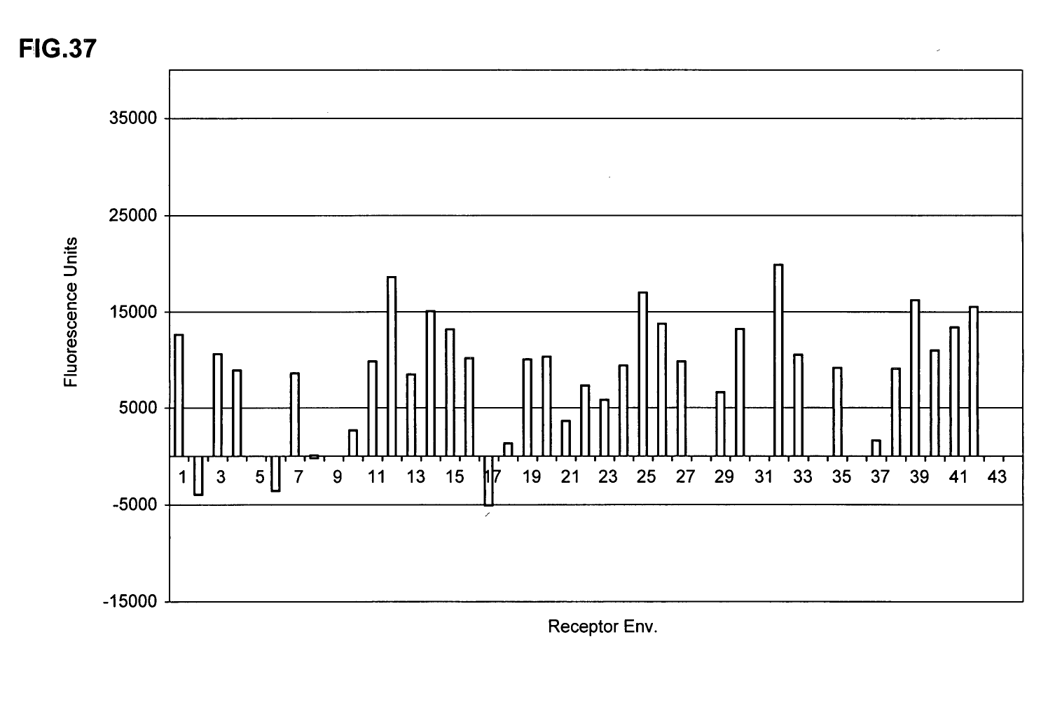

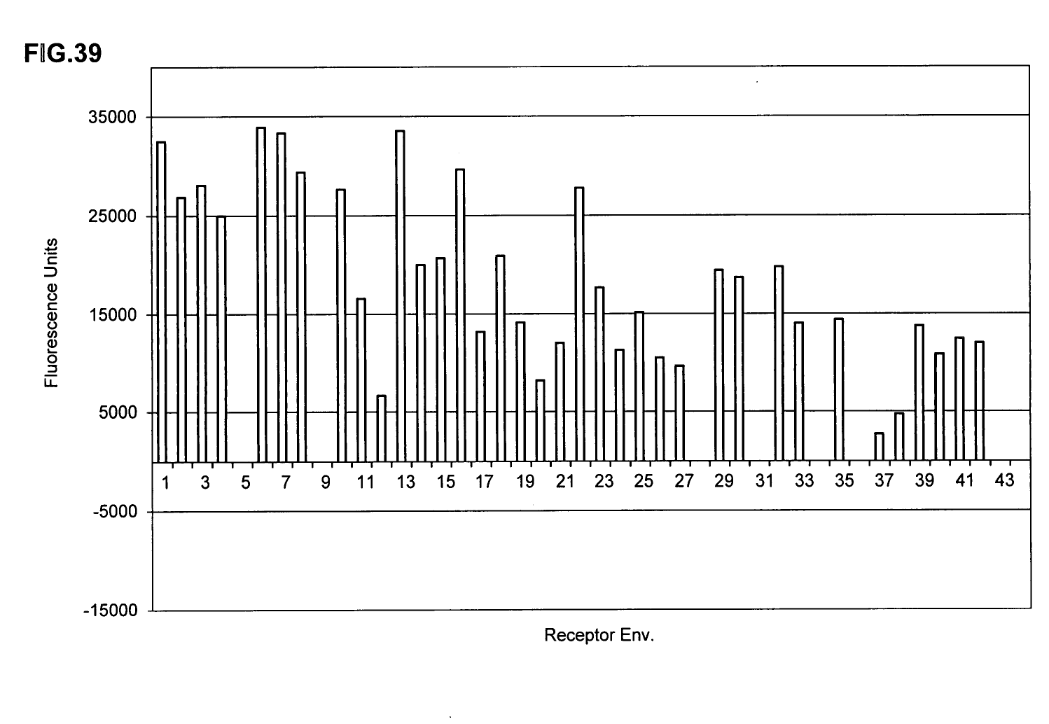

FIG. 39 can be compared to FIG. 37. The fluorescence signals plotted in FIG. 37 resulted from binding to reversibly immobilized building blocks on a support at 23° C. The fluorescence signals plotted in FIG. 39 resulted from binding to covalently immobilized building blocks on a support at 23° C. These figures compare the same combinations of building blocks in the same relative positions, but immobilized in two different ways.

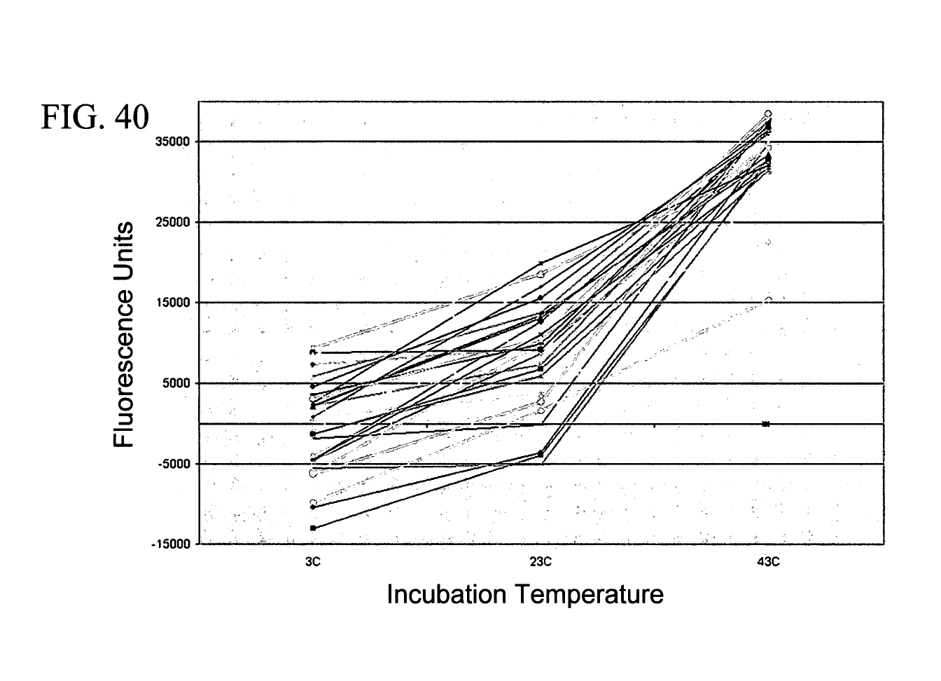

FIG. 40 illustrates the changes in fluorescence signal from individual combinations of building blocks at 4° C., 23° C., or 44° C. This graph illustrates that at least one combination of building blocks (candidate artificial receptor) exhibited a signal that remained constant as temperature increased. At least one candidate artificial receptor exhibited an approximately linear increase in signal as temperature increased. Such a linear increase indicates normal temperature effects on binding. The candidate artificial receptor with the lowest binding signal at 4° C. became one of the best binders at 44° C. This indicates that rearrangement of the building blocks of this receptor above the phase transition for the lipophilic lawn produced increased binding. Other receptors characterized by greater changes in binding between 23° C. and 44° C. (compared to between 4° C. and 23° C.) also underwent dynamic affinity optimization.

### CONCLUSIONS

This experiment demonstrated that an array including reversibly immobilized building blocks binds a protein substrate, like an array with covalently immobilized building blocks. The binding increased nonlinearly as temperature increased, indicating that movement of the building blocks increased binding. The candidate artificial receptors demonstrated improved binding upon mobilization of the building blocks.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A method of making an array comprising reversibly immobilized building blocks, the method comprising:
forming a plurality of spots on a solid support, the spots comprising a plurality of building blocks;
reversibly immobilizing building blocks on the solid support in the spots.

2. The method of claim 1, further comprising mixing the plurality of building blocks and employing the mixture in forming the plurality of spots.

3. The method of claim 1, wherein forming comprises piezoelectric spotting, pin spotting, electromagnetic spotting, or photolithography.

4. The method of claim 1, wherein the solid support comprises a glass plate or microscope slide.

5. A method of making a receptor surface, the method comprising:
forming a region on a solid support, the region comprising a plurality of building blocks;
reversibly immobilizing the plurality of building blocks on the solid support in the region.

6. The method of claim 5, further comprising mixing the plurality of building blocks and employing the mixture in forming the receptor surface.

7. The method of claim 5, wherein the solid support comprises a tube, plate, or well.

8. A method of making an artificial receptor, the method comprising:
forming a region on a support, the region comprising a plurality of building blocks;
reversibly immobilizing building blocks on the support in the region.

9. The method of claim 8, wherein the region is a spot.

10. A composition comprising:
a support, a functionalized lawn, and a plurality of building blocks;
the functionalized lawn being coupled to the support;
building blocks being reversibly immobilized on the lawn.

11. The composition of claim 10, wherein the functionalized lawn comprises a first covalent bonding moiety and the building block comprises a second covalent bonding moiety, the first and second covalent bonding moieties forming a readily reversible covalent bond.

12. The composition of claim 11, wherein:
the first covalent bonding moiety comprises an amine nitrogen and the second covalent bonding moiety comprises a carbonyl carbon;
the first covalent bonding moiety comprises a carbonyl carbon and the second covalent bonding moiety comprises an amine nitrogen;
or combination thereof.

13. The composition of claim 10, wherein the functionalized lawn comprises a first charged moiety and the building block comprises a second charged moiety, the first and second charged moieties having opposite charges.

14. The composition of claim 13, wherein:
the first charged moiety comprises a carboxylate and the second charged moiety comprises an ammonium;
the first charged moiety comprises an ammonium and the second charged moiety comprises a carboxylate;
or combination thereof.

15. The composition of claim 13, wherein the first charged moiety comprises amine, quaternary ammonium, sulfonium, phosphonium, ferrocene, or mixture thereof.

16. The composition of claim 13, wherein the second charged moiety comprises carboxylate, alkoxylate, phenol substituted with strongly electron withdrawing group, phosphate, phosphonate phosphinate, sulphate, sulphonates, thiocarboxylate, hydroxamic acid, or mixture thereof.

17. The composition of claim 10, wherein the functionalized lawn comprises a first lipophilic moiety and the building block comprises a second lipophilic moiety.

18. The composition of claim 17, wherein the first and second lipophilic moieties comprise independently branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof.

19. The composition of claim 17, wherein:
the functionalized lawn comprises a first lipophilic moiety and a first covalent bonding moiety; and the building block comprises a second lipophilic moiety and a second covalent bonding moiety;
the functionalized lawn comprises a first lipophilic moiety and a first charged moiety; and the building block comprises a second lipophilic moiety and a second charged moiety;
or combination thereof.

20. The composition of claim 10, comprising a plurality of spots on the support, the spots comprising a plurality of building blocks.

21. The composition of claim 20, wherein the spots are configured in an array.

22. The composition of claim 21, wherein the array comprises more than 1 million spots.

23. The composition of claim 20, wherein the spots comprise 2, 3, 4, 5, or 6 building blocks.

24. The composition of claim 20, wherein the support comprises a solid support.

25. The composition of claim 24, comprising a plurality of spots on a surface of the solid support.

26. The composition of claim 24, comprising a functionalized glass support.

27. The composition of claim 10, comprising a candidate artificial receptor, a lead artificial receptor, a working artificial receptor, or a combination thereof.

28. The composition of claim 27, wherein the artificial receptor comprises 2, 3, 4, 5, or 6 different building blocks.

29. A heterogeneous building block array comprising:
a support, a functionalized lawn, and a plurality of building blocks;
the functionalized lawn being coupled to the support;
a plurality of regions on the support;
the regions comprising a plurality of building blocks; and
building blocks being reversibly immobilized on the lawn.

30. A composition comprising:
a surface, a functionalized lawn, and a plurality of building blocks;
the functionalized lawn being coupled to the surface;
a region on the surface comprising a plurality of building blocks;
building blocks being reversibly immobilized on the lawn.

31. An article of manufacture comprising:
a support, a functionalized lawn reagent, and a plurality of building blocks;
the functionalized lawn being configured to be coupled to the support;
the plurality of building blocks being configured to be reversibly coupled to the lawn.

32. The article of manufacture of claim 31, wherein the functionalized lawn reagent comprises a first covalent bonding moiety and the building block comprises a second covalent bonding moiety.

33. The article of manufacture of claim 31, wherein the functionalized lawn reagent comprises a first charged moiety and the building block comprises a second charged moiety, the first and second charged moieties having opposite charges.

34. The article of manufacture of claim 31, wherein the functionalized lawn reagent comprises a first lipophilic moiety and the building block comprises a second lipophilic moiety.

35. The article of manufacture of claim 31, comprising a functionalized glass support.

36. A method of using an artificial receptor comprising:
contacting a reversibly immobilized heterogeneous molecular array with a test ligand;
the array comprising:
a support, a functionalized lawn, and a plurality of building blocks;
the functionalized lawn being coupled to the support;
a plurality of regions on the support;
the regions comprising a plurality of building blocks; and
the plurality of building blocks being reversibly immobilized on the lawn;
shuffling building blocks in one or more regions;
detecting binding of a test ligand to one or more regions; and
selecting one or more of the binding regions as the artificial receptor;
wherein the building blocks in the array define a first set of building blocks, and the plurality of building blocks in the one or more binding regions defines one or more selected binding combination of building blocks.

37. The method of claim 36, wherein the artificial receptor comprises a lead artificial receptor.

38. The method of claim 36, wherein:
the functionalized lawn comprises a first covalent bonding moiety and the building block comprises a second covalent bonding moiety, the first and second covalent bonding moieties forming a readily reversible covalent bond; and
shuffling comprises contacting one or more regions to be shuffled with a composition comprising reagent promoting cleavage of the readily reversible covalent bond;

39. The method of claim 36, wherein the readily reversible covalent bond comprises an acetal or ketal bond and the reagent comprises pH about 1 to about 4.

40. The method of claim 36, wherein:
the functionalized lawn comprises a first charged moiety and the building block comprises a second charged moiety, the first and second charged moieties having opposite charges; and
shuffling comprises contacting one or more regions to be shuffled with a composition comprising reagent promoting separation of the first and second charged moieties;

41. The method of claim 40, wherein the reagent comprises salt concentration of about 0.1 to about 1 M.

42. The method of claim 36, wherein:
the functionalized lawn comprises a first lipophilic moiety and the building block comprises a second lipophilic moiety; and
shuffling comprises contacting one or more regions to be shuffled with a composition comprising lipophilic reagent;

43. The method of claim 42, wherein the lipophilic reagent comprises organic solvent, surfactant, or mixture thereof.

44. The method of claim 42, wherein the organic solvent comprises acetonitrile, acetic acid, an alcohol, tetrahydrofuran (THF), dimethylformamide (DMF), hydrocarbon solvent, acetone, chloroform, methylene chloride, or mixture thereof.

45. The method of claim 36, comprising:
shuffling before detecting;
detecting before shuffling;
shuffling, then detecting, then shuffling again;
contacting, then shuffling, then contacting again; or
combinations thereof.

46. The method of claim 36, further comprising
determining the combinations of building blocks in one or more of the binding regions;
developing, based on the combinations determined, one or more developed sets of building blocks distinct from those in the one or more selected combinations of building blocks;
exchanging into one or more of the regions one or more of the developed sets of building blocks;
detecting binding of a test ligand to one or more of the exchanged regions; and
selecting one or more of the spots of the second heterogeneous molecular array as the artificial receptor.

47. The method of claim 46, wherein the artificial receptor comprises a lead artificial receptor.

48. The method of claim 46, further comprising varying the structure of the lead artificial receptor to increase binding speed or binding affinity of the test ligand.

49. The method of claim 46, wherein the first set of building blocks comprises a subset of a larger set of building blocks.

50. The method of claim 49, wherein the first set of building blocks comprises a subset of a larger set of building blocks, the second subset of building blocks defines a subset of the larger set of building blocks, and the first subset is not equivalent to the second subset.

51. The method of claim 46, comprising:
shuffling before detecting;
detecting before shuffling;
shuffling, then detecting, then shuffling again;
contacting, then shuffling, then contacting again;
exchanging before detecting;
detecting before exchanging;
exchanging, then detecting, then exchanging again;
contacting, then exchanging, then contacting again;
shuffling before exchanging;
exchanging before shuffling; or
combinations thereof.

52. The method of claim 36, wherein the regions comprise 2, 3, or 4 building blocks.

53. The method of claim 36, further comprising:
identifying the plurality of building blocks making up the artificial receptor;
coupling the identified plurality of building blocks to a scaffold molecule;
evaluating the scaffold artificial receptor for binding of the test ligand.

54. The method of claim 53, wherein:
coupling comprises making a plurality of positional isomers of the building blocks on the scaffold;
evaluating comprises comparing the plurality of the scaffold positional isomer artificial receptors; and
selecting one or more of the scaffold positional isomer artificial receptors as lead or working artificial receptor.

55. The method of claim 36, further comprising applying the test ligand to one or more regions that function as controls for validating or evaluating binding to an artificial receptor.

56. The method of claim 53, wherein the control region comprises no building block, only a single building block, only functionalized lawn, or a combination thereof.

57. A method of using an artificial receptor comprising:
contacting a first reversibly immobilized heterogeneous molecular array with a test ligand;
the array comprising:
a support, a functionalized lawn, and a plurality of building blocks;
the functionalized lawn being coupled to the support;
a plurality of regions on the support;
the regions comprising a plurality of building blocks; and
the plurality of building blocks being reversibly immobilized on the lawn;
exchanging building blocks onto or off of the support;
detecting binding of a test ligand to one or more regions; and
selecting one or more of the binding regions as the artificial receptor;
wherein the building blocks in the array define a first set of building blocks, and the plurality of building blocks in the one or more binding regions defines one or more selected binding combination of building blocks.

58. The method of claim 57, wherein the artificial receptor comprises a lead artificial receptor.

59. The method of claim 57, wherein exchanging comprises contacting one or more regions with added building block and reversibly immobilizing the added building block in the region.

60. The method of claim 57, wherein exchanging comprises contacting one or more regions with reagent promoting release of reversibly immobilized building block and removing released building block.

61. The method of claim 57, wherein exchanging comprises:
contacting one or more regions with reagent promoting release of reversibly immobilized building block and removing released building block; and
contacting one or more regions with added building block and reversibly immobilizing the added building block in the region.

62. The method of claim 57, further comprising shuffling building blocks in one or more regions.

63. The method of claim 62, comprising:
shuffling before detecting;
detecting before shuffling;
shuffling, then detecting, then shuffling again;
contacting, then shuffling, then contacting again;
exchanging before detecting;
detecting before exchanging;
exchanging, then detecting, then exchanging again;
contacting, then exchanging, then contacting again; or
shuffling before exchanging;
exchanging before shuffling; or
combinations thereof.

64. The method of claim 57, comprising:
exchanging before detecting;
detecting before exchanging;
exchanging, then detecting, then exchanging again;
contacting, then exchanging, then contacting again; or
combinations thereof.

65. The method of claim 57, further comprising
determining the combinations of building blocks in the one or more binding regions;
developing, based on the combinations determined, one or more developed sets of building blocks distinct from those in the one or more selected combinations of building blocks;
exchanging into one or more of the regions the one or more developed sets of building blocks;
detecting binding of a test ligand to one or more of the exchanged regions; and
selecting one or more of the regions of the second heterogeneous molecular array as the artificial receptor.

66. The method of claim 65, wherein the artificial receptor comprises a lead artificial receptor.
